(19) United States
(12) Patent Application Publication
(10) Pub. No.: US 2007/0010529 A1
(43) Pub. Date: Jan. 11, 2007

(54) Nitrogenous heterocyclic compounds and medical use thereof

(76) Inventors: Kanji Takahashi, Osaka (JP); Naoki Sumino, Osaka (JP); Shingo Yamamoto, Mishima (JP); Masafumi Sugitani, Mishima (JP); Akihiko Uegaki, Mishima (JP); Shingo Nakatani, Mishima (JP); Naoki Matsunaga, Mishima (JP); Takayuki Inukai, Mishima (JP)

Correspondence Address: WENDEROTH, LIND & PONACK, L.L.P., 2033 K STREET N. W., SUITE 800, WASHINGTON, DC, 20006-1021, US

(21) Appl. No.: 10/557,352

(22) PCT Filed: May 18, 2004
(86) PCT No.: PCT/JP04/07070

§ 371 Date: Dec. 30, 2005

(30) Foreign Application Priority Data
May 19, 2003 (JP) 2003-141042
Sep. 29, 2003 (JP) 2003-338389
Apr. 2, 2004 (JP) 2004-110572

(51) Int. Cl. A61K 31/496; A61K 31/454; C07D 403/02; A61K 31/4152

(52) U.S. Cl. 514/254.05; 514/406; 548/368.4; 514/326; 544/371; 546/211

(57) ABSTRACT

The present invention relates to a compound represented by formula (I): 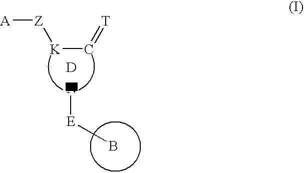 (wherein all the symbols have the same meanings as defined in the above description) and a production method and use thereof. The compounds represented by formula (I), or its salt, N-oxide or solvate, or a prodrug thereof have p38 MAP kinase inhibitory activity, and are useful in the prevention and/or treatment of those diseases that are supposedly caused or deteriorated by abnormal production of cytokines including inflammatory cytokine or chemokine, or by over response thereto, namely cytokine-mediated diseases such as inflammatory diseases, respiratory diseases, cardiovascular disease, central nervous system diseases, and the like.

### TECHNICAL FIELD

The present invention relates to a nitrogen-containing heterocyclic compound having p38 MAP kinase inhibitory activity useful as medicines, and production method and use thereof.

### BACKGROUND ART

p38 mitogen-activated protein kinase (p38α/Mpk2/RK/SAPK2a/CSBP)(hereinafter referred to as “p38 MAP kinase”) was cloned as an enzyme which induces tyrosine phosphorylation in monocytes after stimulation with lipopolysaccharide (LPS)[Nature, 372, 739 (1994)], and is activated by various extracellular stimuli (physical stimuli: osmotic shock, heat shock, UV irradiation; chemical stimuli: endotoxin, hydrogen peroxide, arsenic trioxide, inflammatory cytokine and growth factor). Also, since p38 MAP kinase relates to the production of cytokines including inflammatory cytokines (e.g. tumor necrosis factor-α (TNF-α), interleukin 1 (IL-1), IL-6 and IL-8) and chemokines, it is strongly suggested that there is an association between the activation of this enzyme and diseases. Therefore, it is expected that suppression of p38 MAP kinase activation would have an improvement effect on various disease symptoms represented by inflammatory diseases.

Accordingly, a p38 MAP kinase inhibitor is expected to be useful in the prevention and/or treatment of those diseases that are supposedly caused or deteriorated by abnormal production of cytokines including inflammatory cytokine or chemokine, or by over response thereto, namely cytokine-mediated diseases such as various inflammatory diseases, for example, inflammation, dermatitis, atopic dermatitis, hepatitis, nephritis, glomerulonephritis, pancreatitis, psoriasis, gout, Addison's disease, arthrititis (e.g. articular rheumatism, osteoarthritis, rhumatoid spondylitis, gouty arthritis, synovitis, etc.), inflammatory ocular diseases, inflammatory pulmonary diseases (e.g. chronic pneumonia, silicosis, pulmonary sarcoidosis, pulmonary tuberculosis, adult respiratory distress syndrome (ARDS), severe acute respiratory syndrome (SARS), etc.), inflammatory bowel diseases (e.g. Crohn's disease, ulcerative colitis, etc.), allergic diseases (e.g. allergic dermatitis, allergic rhinitis, etc.), autoimmune disease, autoimmune hemolytic anemia, systemic lupus eryhtematosus, rheumatism, Castleman's disease, immune rejection accompanying transplantation (e.g. graft versus host reaction, etc.), and the like; central nervous system disorders, for example, central neuropathy (e.g. cerebrovascular disease such as cerebral hemorrhage and cerebral infarction, head trauma, spinal cord injury, cerebral edema, multiple sclerosis, etc.), neuronal degeneration (e.g. Alzheimer's disease, Parkinson's disease, amyotrophic lateral sclerosis (ALS), AIDS dementia, etc.), meningitis, Creutzfeldt-Jakob syndrome, and the like; respiratory diseases, for example, asthma, chronic obstructive pulmonary disease (COPD), and the like; cardiovascular diseases, for example, angina pectoris, heart failure, congestive heart failure, acute heart failure, chronic heart failure, myocardial infarction, acute myocardial infarction, myocardial infarction prognosis, atrial myxoma, arteriosclerosis, hypertension, dialysis-induced hypotension, thrombosis, disseminated intravascular coagulation (DIC), reperfusion injury, restenosis after percutaneous transluminal coronary angioplasty (PTCA), and the like; urinary diseases, for example, renal failure, and the like; metabolic diseases or endocrine diseases, for example, diabetes, and the like; bone diseases, for example, osteoporosis, and the like; cancerous diseases, for example, malignant tumor (e.g. tumor growth and metastasis), multiple myeloma, plasma cell leukemia, carcinemia, and the like; and infectious diseases, for example, viral infection (e.g. cytomegalovirus infection, influenza virus infection, herpes virus infection, corona virus infection, etc.), cachexia associated with infections, cachexia caused by acquired immune deficiency syndrome (AIDS), toxemia (e.g. sepsis, septic shock, endotoxin shock, gram negative bacterial sepsis, toxic shock syndrome, severe acute respiratory syndrome (SARS) accompanying virus infection, and the like.

DE10002509 discloses that compounds shown by formula (X) are useful as IL-12 inhibitors: 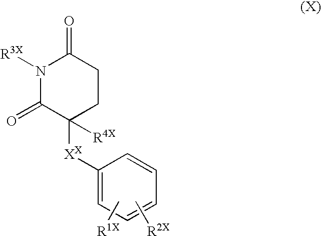
wherein R^{1X} and R^{2X} each represents a hydrogen atom, a chlorine atom, a fluorine atom, a hydroxy group, or the like;
R^{3X} represents a hydrogen atom, a hydroxy group, or —CH₂NR^{6X}R^{7X};
R^{6X} and R^{7X}, taken together, form a pyrrolidine ring, a piperidine ring, a morpholine ring, or the like;
R^{4X} represents a hydrogen atom, a C1-3 alkyl group, a fluorine atom, trifluoromethyl, or difluoromethyl;
X^{X} represents —(CH₂)_{nX}—NR^{8X}—, —(CH₂)ₙₓ—S—, —(CH₂)_{qx}—, or the like, provided that necessary ones of the symbols in the groups are extracted.

On the other hand, WO 00/043384 discloses that compounds represented by formula (Y) are useful as anti-inflammatory agents: 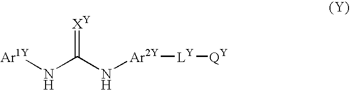
wherein Ar^{1Y} represents an optionally substituted heterocyclic ring;

Ar^{2Y} represents an optionally substituted phenyl group, or the like;

L^{Y} represents a C1-10 alkylene group, or the like;

Q^{Y} represents a phenyl group optionally substituted by a halogen atom, or the like;

X^{Y} represents an oxygen atom or a sulfur atom; provided that necessary ones of the symbols in the groups are extracted.

Further, WO 03/043988 discloses that compounds represented by formula (A) or their non-toxic salts are useful as P38 MAP kinase inhibitors: 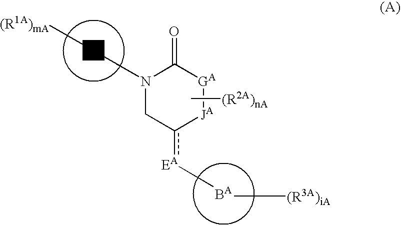
wherein A^{A} represents a C5-10 mono- or bi-carbocyclic ring, or a 5- or 10-membered mono- or bi-heterocyclic ring containing 1 to 5 nitrogen atom(s), 1 to 2 oxygen atom(s) and/or 1 sulfur atom;

R^{1A} represents (1) C1-8 alkyl, (2) C2-8 alkenyl, (3) C2-8 alkynyl, (4) halogen, (5) —OR^{4A}, (6) —NR^{5A}R^{6A}, (7) —NR^{7A}COR^{8A},
(8) —CONR9AR^{10A}, (9) —COOR^{11A}(10) —SO₂NR^{12A}R^{13A}, (11) —NR^{14A}SO₂R^{15A}, (12) —SR^{16A}, (13) —S(O)R^{17A}, (14) —SO₂R^{18A}, (15) —NR^{22A}COOR^{23A}, (16) —NR^{24A}CONR^{25A}R^{26A}, (17) —COR^{27A}, (18) nitro, (19) cyano, (20) trifluoromethyl, (21) trifluoromethoxy, (22) Cyc1^{A}, or the like;

R^{4A}-R^{18A} and R^{22A}-R^{21A} each independently represent a hydrogen atom, C1-8 alkyl, Cycl^{A}, or the like;

Cyc1^{A} represents a C5-10 mono- or bi-cabocyclic ring (provided that said carbocyclic ring may be substituted with one to five of R^{48A});

R^{48A} represents C1-8 alkyl, halogen, nitro, cyano, or the like;

R^{2A} represents C1-8 alkyl, —OR^{20A}, —NR^{64A}R^{65A}, —COOR^{66A}, —CONR^{67A}R^{68A}, —NR^{69A}COR^{70A}, —SO₂R^{71A}, —SO₂NR^{72A}R^{73A}, —NR^{74A}SO₂R^{75A}, —NR^{76A}COOR^{77A}, Cyc2^{A}, or the like;

R^{20A} and R^{64A}—R^{77A} each independently represents hydrogen, C1-8 alkyl, Cyc2^{A}, or the like;

Cyc2^{A} represents a C5-6 monocarbocyclic ring or the like (provided that said carbocyclic ring may be substituted by one to five substituent(s) such as C1-8 alkoxy, halogen or the like);

G^{A} and J^{A} each independently represent a carbon, nitrogen, oxygen, or sulfur atom;

E^{A} represents C1-4 alkylene, —O—, —S—, or the like (provided that said C1-4 alkylene may be substituted by 1 to 5 substituent(s) such as C1-8 alkoxy, halogen, hydroxy, and the like);

B^{A} represents a C5-10 mono- or bi-carbocyclic ring, or a C5-10 mono- or bi-heterocyclic ring containing 1 to 4 nitrogen atom(s), 1 to 2 oxygen atom(s) and/or 1 sulfur atom;

R^{3A} represents C1-8 alkyl, C2-8 alkenyl, C2-8 alkynyl, halogen, —OR^{81A}, —NR^{82A}R^{83A}, —NR^{84A}COR^{85A}, —CONR^{86A}R^{87A}, —COOR^{88A}, SO₂NR^{89A}R^{90A}—NR^{91A}SO₂R^{92A}, —SR^{93A}, —S(O)R^{94A}, —SO₂R^{95A}, —NR^{96A}COOR^{97A}, —NR^{98A}CONR^{99A}R^{100A}, —OCONR^{101A}R^{102A}, nitro, cyano, trifluoromethyl, trifluoromethoxy, Cyc4^{A}, or the like;

R^{81A}-R^{102A} each independently represents hydrogen, C1-8 alkyl, Cyc4^{A}, or the like;

Cyc4^{A} represents a C5-10 mono- or bi-carbocyclic ring (provided that said carbocyclic ring may be substituted by 1 to 5 substituent(s) such as C1-8 alkoxy, halogen and the like);

mA represents 0 or an integer of 1 to 5;

nA represents 0 or an integer of 1 to 7;

iA represents 0 or an integer of 1 to 12, provided that necessary ones of the symbols in the groups are extracted.

### DISCLOSURE OF THE INVENTION

Previously known p38 MAP kinase inhibitors are known to have drawbacks including cytochrome P450 (hereinafter referred to as CYP) inhibitory activity and CYP-inducing action. CYP is one of the important enzymes involved in the metabolism of medicine. In the clinical field, since two or more medicines are often used simultaneously, increase or decrease of CYP due to CYP inhibitory activity or CYP-inducing action in a living body may cause unexpected potentiation or reduction in the effect of the objective medicines or the combination drugs. Accordingly, p38 MAP kinase inhibitors having CYP inhibitory activity or CYP-inducing action may not become a safe medicine. The technical problem to be solved by the present invention is to develop p38 MAP kinase inhibitors useful for prevention and/or treatment of various diseases typically such as inflammatory diseases, which are excellent in oral absorption, do not affect CYP, can be used with other medicines, and can be safely administered.

The present inventors have intensively studied to find a compound inhibiting activation of p38 MAP kinase and not affecting CYP, which can become a safe therapeutic agent for various diseases typically such as inflammatory disease. As a result of such studies, the present inventors have found that novel heterocyclic compounds represented by formula (I) described below can attain such purpose, and thus the present invention has been completed based on these findings. That is, the present invention relates to:

(1) a compound represented by formula (I): 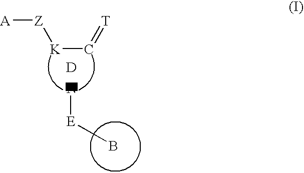
wherein A represents a hydrogen atom, an optionally substituted cyclic group, an optionally substituted aliphatic hydrocarbon group or an optionally protected amino group;

ring B represents an optionally substituted cyclic group;

E represents a spacer having 1 to 4 atom(s) in its main chain;

K represents a carbon atom or a nitrogen atom;

Z represents a bond, an oxygen atom, a sulfur atom, —NR^{Z}— or —N(SO₂R^{ZZ})—;

R^{Z} represents a hydrogen atom, an optionally substituted cyclic group or an optionally substituted aliphatic hydrocarbon group;

R^{ZZ} represents an optionally substituted cyclic group or an optionally substituted aliphatic hydrocarbon group;

—C(=T)- represents —C(═O)—, —C(═S)— or an optionally substituted methylene group; and

ring D represents an optionally further substituted heterocyclic ring containing at least one nitrogen atom;

or its salt, N-oxide, or solvate, or a prodrug thereof,

(2) the compound according to the above (1), wherein Z is a bond,

(3) the compound according to the above (1), wherein Z is an oxygen atom,

(4) the compound according to the above (1), wherein —C(=T)- is —C(═O)—,

(5) the compound according to the above (1), wherein —C(=T)- is an optionally substituted methylene group,

(6) the compound according to the above (1), wherein A is an optionally substituted 5- to 10-membered cyclic group,

(7) the compound according to the above (1), wherein A is an optionally substituted C1-8 aliphatic hydrocarbon group or an optionally protected amino group,

(8) the compound according to the above (1), wherein the substituent in the A is —NR^{a1}CONR^{a2}R^{a3} in which R^{a1}, R^{a2} and R^{a3} each independently represents a hydrogen atom, an optionally substituted C₁₋₈ alkyl group, an optionally substituted 5- to 10-membered carbocyclic ring, or an optionally substituted 5- to 10-membered heterocyclic ring,

(9) the compound according to the above (1), wherein A is

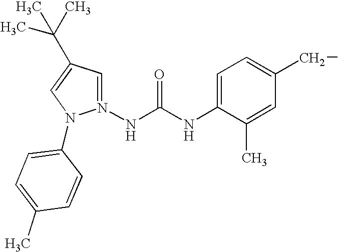

(10) the compound according to the above (1), wherein the substituent in the A is —CH₂—CONR^{a1}R^{a2} in which R^{a1} and R^{a2} each independently represents a hydrogen atom, an optionally substituted C1-8 alkyl group, an optionally substituted 5- to 10-membered carbocyclic ring, or an optionally substituted 5- to 10-membered heterocyclic ring,

(11) the compound according to the above (1), wherein the ring B is an optionally substituted 5- to 10-membered cyclic group,

(12) the compound according to the above (1), wherein the substituent on the ring B is —NR^{a1}CONR^{a2}R^{a3} in which R^{a1}, R^{a2} and R^{a3} each independently represents a hydrogen atom, an optionally substituted C₁₋₈ alkyl group, an optionally substituted 5- to 10-membered carbocyclic ring, or an optionally substituted 5- to 10-membered heterocyclic ring,

(13) the compound according to the above (1), wherein the ring B is

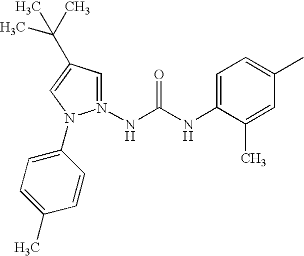

(14) the compound according to the above (1), wherein the substituent on the ring B is —CH₂—CONR^{a1}R^{a2} in which R^{a1} and R^{a2} each independently represents a hydrogen atom, an optionally substituted C₁₋₈ alkyl group, an optionally substituted 5- to 10-membered carbocyclic ring, or an optionally substituted 5- to 10-membered heterocyclic ring,

(15) the compound according to the above (1), wherein K is a nitrogen atom,

(16) the compound according to the above (1), wherein K is a carbon atom,

(17) the compound according to the above (1), wherein the ring D is an optionally further substituted 6-membered heterocyclic ring containing at least one nitrogen atom,

(18) the compound according to the above (4), wherein 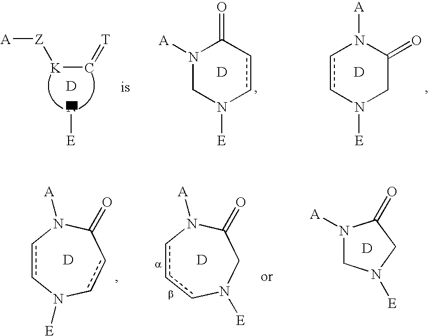
in which [symbol] is a single bond or a double bond, and other symbols have the same meanings as defined in the above (1), provided that the α-bond and β-bond do not simultaneously represent a double bond,

(19) the compound according to the above (5), wherein 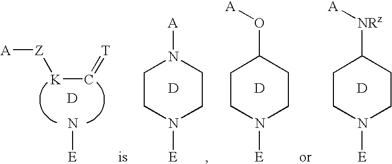
in which all symbols have the same meanings as defined in claim 1,

(20) the compound according to the above (1), which is selected from the group consisting of:
1) N-[3-t-butyl-1-(4-methylphenyl)-1H-pyrazol-5-yl]-N′-(3-{[4-(2-methylbenzoyl)-1-piperazinyl]methyl}phenyl)urea,
2) N-[3-t-butyl-1-(4-methylphenyl)-1H-pyrazol-5-yl]-N′-(2-methyl-4-{[4-(2-methylbenzoyl)-1-piperazinyl]methyl}phenyl)urea,
3) N-[3-t-butyl-1-(4-methylphenyl)-1H-pyrazol-5-yl]-N′-(3-chloro-4-{[4-(2-methylbenzoyl)-1-piperazinyl]methyl}phenyl)urea,
4) N-[3-t-butyl-1-(4-methylphenyl)-1H-pyrazol-5-yl]-N′-[4-({4-[3-(dimethylamino)benzoyl]piperazin-1-yl}methyl)-2-methylphenyl]urea,
5) N-[3-t-butyl-1-(4-methylphenyl)-1H-pyrazol-5-yl]-N′-{4-[(4-{4-[(E)-(hydroxyimino)methyl]benzoyl}piperazin-1-yl)methyl]-2-methylphenyl}urea,
6) N-[3-t-butyl-1-(4-methylphenyl)-1H-pyrazol-5-yl]-N′-{4-[(4-{4-[(dimethylamino)methyl]benzoyl}piperazin-1-yl)methyl]-2-methylphenyl}urea,
7) N-[3-t-butyl-1-(4-methylphenyl)-1H-pyrazol-5-yl]-N′-(2,6-dimethyl-4-{[4-(2-methylbenzoyl)piperazin-1-yl]methyl}phenyl)urea,
8) N-[3-t-butyl-1-(4-methylphenyl)-1H-pyrazol-5-yl]-N′-(2-fluoro-4-{[4-(2-methylbenzoyl)piperazin-1-yl]methyl}phenyl)urea,
9) N-[3-t-butyl-1-(4-methylphenyl)-1H-pyrazol-5-yl]-N′-{2-methyl-4-[(4-{4-[(4-methylpiperazin-1-yl)methyl]benzoyl}piperazin-1-yl)methyl]phenyl}urea,
10) N-[3-t-butyl-1-(4-methylphenyl)-1H-pyrazol-5-yl]-N′-(2-methyl-5-{[4-(2-methylbenzoyl)piperazin-1-yl]methyl}phenyl)urea,
11) N-{4-[(1-benzoylpiperidin-4-yl)oxy]-2-methylphenyl}-N′-[3-t-butyl-1-(4-methylphenyl)-1H-pyrazol-5-yl]urea,
12) N-[3-t-butyl-1-(4-methylphenyl)-1H-pyrazol-5-yl]-N′-{4-[(4-{4-[(dimethylamino)methyl]benzoyl}piperazin-1-yl)methyl]-2-fluorophenyl}urea,
13) N-[3-t-butyl-1-(4-methylphenyl)-1H-pyrazol-5-yl]-N′-{2-fluoro-4-[(4-{4-[(4-methylpiperazin-1-yl)methyl]benzoyl}piperazin-1-yl)methyl]phenyl}urea,
14) N-[3-t-butyl-1-(4-methylphenyl)-1H-pyrazol-5-yl]-N′-{3-[(4-{4-[(dimethylamino)methyl]benzoyl}piperazin-1-yl)methyl]-2-methylphenyl}urea,
15) N-[3-t-butyl-1-(4-methylphenyl)-1H-pyrazol-5-yl]-N′-{2-methyl-3-[(4-{4-[(4-methylpiperazin-1-yl)methyl]benzoyl}piperazin-1-yl)methyl]phenyl}urea,
16) N-[3-t-butyl-1-(4-methylphenyl)-1H-pyrazol-5-yl]-N′-{5-[(4-{4-[(dimethylamino)methyl]benzoyl}piperazin-1-yl)methyl]-2-methylphenyl}urea,
17) N-[3-t-butyl-1-(4-methylphenyl)-1H-pyrazol-5-yl]-N′-{2-methyl-5-[(4-{4-[(4-methylpiperazin-1-yl)methyl]benzoyl}piperazin-1-yl)methyl]phenyl}urea,
18) N-[3-t-butyl-1-(4-methylphenyl)-1H-pyrazol-5-yl]-N′-(2-fluoro-5-{[4-(2-methylbenzoyl)piperazin-1-yl]methyl}phenyl)urea,
19) N-[3-t-butyl-1-(4-methylphenyl)-1H-pyrazol-5-yl]-N′-(2-chloro-5-{[4-(2-methylbenzoyl)piperazin-1-yl]methyl}phenyl)urea,
20) N-[3-t-butyl-1-(4-methylphenyl)-1H-pyrazol-5-yl]-N′-(2-methoxy-5-{[4-(2-methylbenzoyl)piperazin-1-yl]methyl}phenyl)urea,
21) N-[3-t-butyl-1-(4-methylphenyl)-1H-pyrazol-5-yl]-N′-{2-methyl-4-[(1-{4-[(4-methylpiperazin-1-yl)methyl]benzoyl}piperidin-4-yl)oxy]phenyl}urea,
22) N-[3-t-butyl-1-(4-methylphenyl)-1H-pyrazol-5-yl]-N′-{2-fluoro-5-[(4-{4-[(4-methylpiperazin-1-yl)methyl]benzoyl}piperazin-1-yl)methyl]phenyl}urea,
23) N-[3-t-butyl-1-(4-methylphenyl)-1H-pyrazol-5-yl]-N′-{2-methyl-4-[(4-{3-[(4-methylpiperazin-1-yl)methyl]benzoyl}piperazin-1-yl)methyl]phenyl}urea,
24) N-[3-t-butyl-1-(4-methylphenyl)-1H-pyrazol-5-yl]-N′-{2-chloro-5-[(4-{4-[(4-methylpiperazin-1-yl)methyl]benzoyl}piperazin-1-yl)methyl]phenyl}urea, and
25) N-[3-t-butyl-1-(4-methylphenyl)-1H-pyrazol-5-yl]-N′-{2-methyl-4-[(4-{2-[(4-methylpiperazin-1-yl)methyl]benzoyl}piperazin-1-yl)methyl]phenyl}urea,

(21) a pharmaceutical composition containing a compound represented by formula (I): 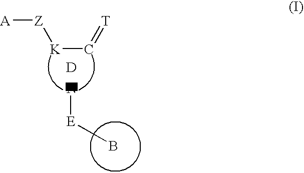
wherein all symbols have the same meanings as defined in the above (1), or its salt, N-oxide or solvate, or a prodrug thereof,

(22) the composition according to the above (21), which is a p38 MAP kinase inhibitor,

(23) the composition according to the above (21), which is a TNF-α production inhibitor,

(24) the composition according to the above (21), which is an agent for prevention and/or treatment of cytokine-mediated disease,
(25) the composition according to the above (24), wherein the cytokine-mediated disease is inflammatory disease, cardiovascular disease, respiratory disease and/or bone disease,
(26) the composition according to the above (24), wherein the cytokine-mediated disease is central nervous system disease, urinary disease, metabolic disease, endocrine disease, infectious disease and/or cancerous disease,
(27) the composition according to the above (25), wherein the inflammatory disease is arthritis rheumatism,
(28) a combination medicine comprising a compound of the above (1), its salt, N-oxide, or solvate, or a prodrug thereof, and one or two or more compound(s) selected from the group consisting of a non-steroidal inflammatory agent, a disease modifying anti-rheumatic agent, an anticytokine protein preparation, a cytokine inhibitor, an immunomodulator, a steroidal agent, an adhesion molecule inhibitor, an elastase inhibitor, a cannabinoid-2 receptor stimulant, a prostaglandin, a prostaglandin synthase inhibitor, a phosphodiesterase inhibitor and a metalloproteinase inhibitor,
(29) a method for prevention and/or treatment of diseases caused by p38 MAP kinase inhibitor in a mammal, which comprises administering an effective amount of a compound of the above (1), or its salt, N-oxide, or solvate, or a prodrug thereof to a mammal,
(30) use of a compound of the above (1), its salt, N-oxide, or solvate, or a prodrug thereof for the preparation of an agent for prevention and/or treatment of diseases caused by p38 MAP kinase, and
(31) a method for producing a compound represented by formula (I), its salt, N-oxide or solvate, or a produrg thereof.

In the description of the present invention, the “aliphatic hydrocarbon group” in the “optionally substituted hydrocarbon group” includes, for example, a “linear or branched aliphatic hydrocarbon group” and the like. For example, the “linear or branched aliphatic hydrocarbon group” includes a “C1-8 aliphatic hydrocarbon group”. Examples of the “C1-8 aliphatic hydrocarbon group” are C1-8 alkyl groups such as methyl, ethyl, propyl, isopropyl, butyl, sec-butyl, t-butyl, pentyl, hexyl, heptyl, octyl, and isomers thereof; C2-8alkenyl groups such as vinyl, propenyl, butenyl, pentenyl, hexenyl, heptenyl, octenyl, butadienyl, pentadienyl, hexadienyl, heptadienyl, octadienyl, hexatrienyl, heptatrienyl, octatrienyl, and isomers thereof; and C2-8 alkynyl groups such as ethynyl, propynyl, butynyl, pentynyl, hexynyl, heptynyl, octynyl, butadiynyl, pentadiynyl, hexadiynyl, heptadiynyl, octadiynyl, hexatriynyl, heptatriynyl, octatriynyl, and isomers thereof.

In the description of the present invention, there is no particular limitation for the “substituent” in the “optionally substituted aliphatic hydrocarbon group” represented by A so long as it can be a substituent. Said “substituent” includes, for example, (1) a substituent selected from a First Group shown below, (2) an optionally substituted 5- to 10-membered carbocyclic ring, and (3) an optionally substituted 5- to 10-membered heterocyclic ring. Here, one to five substituent(s) among these optional substituents may be located at any position where substitution is possible.

<First Group>

(a) halogen atom (e.g. chlorine, bromine, fluorine, iodine atom), (b) —OR^{a1}, (c) —NR^{a1}R^{a2}, (d) —NR^{a1}COR^{a2}, (e) —CONR^{a1}R^{a2}, (f) —COOR^{a1}, (g) —SO₂NR^{a1}R^{a2}, (h) —NR^{a1}SO₂R^{a2}, (i) —SR^{a1}, (j) —S(O)R^{a1}, (k) —SO₂R^{a1}, (l) —NR^{a1}COOR^{a2}, (m) —NR^{a1}CONR^{a2}R^{a3}, (n) —COR^{a1}, (o) nitro, (p) cyano, (q) trifluoromethyl, (r) trifluoromethoxy, (s) —OCONR^{a1}R^{a2}, and (t) oxo [in these groups, R^{a1}, R^{a2} and R^{a3} each independently represents a hydrogen atom, an optionally substituted C1-8 alkyl group, an optionally substituted 5- to 10-membered carbocyclic ring, or an optionally substituted 5- to 10-membered heterocyclic ring].

Here, there is no particular limitation for the “substituent” in the “optionally substituted C1-8 alkyl group” represented by R^{a1}, R^{a2} and R^{a3} so long as it can be a substituent. Said “substituent” includes, for example, (1) a substituent selected from a Second Group shown below, (2) an optionally substituted 5- to 10-membered carbocyclic ring, and (3) an optionally substituted 5- to 10-membered heterocyclic ring. One to five substituent(s) among these optional substituents may be located at any position where substitution is possible.

<Second Group>

(a) —OR^{b1}, (b) —NR^{b1}R^{b2}, (c) —NR^{b1}COR^{b2}, (d) —CONR^{b1}R^{b2}, (e) —COOR^{b1}, (f) —SO₂NR^{b1}R^{b2}, (g) —NR^{b1}SO₂R^{b2}, (h) —CONR^{b1}NR^{b2}R^{b3} and (i) —CONR^{b1}OR^{b2} [in these groups, R^{b1}, R^{b2} and R^{b3} each independently represents a hydrogen atom, an optionally substituted C1-8 alkyl group, an optionally substituted 5- to 10-membered carbocyclic ring, or an optionally substituted 5- to 10-membered heterocyclic ring].

Here, there is no particular limitation for the “substituent” in the “optionally substituted C1-8 alkyl group” represented by R^{b1}, R^{b2} and R^{b3} so long as it can be a substituent. Said “substituent” includes, for example, (1) a substituent selected from a Third Group shown below, (2) an optionally substituted 5- to 10-membered carbocyclic ring, and (3) an optionally substituted 5- to 10-membered heterocyclic ring. One to five substituent(s) among these optional substituents may be located at any position where substitution is possible.

<Third Group>

(a) —OR^{c1} and (b) —NR^{c1}R^{c2} [in these groups, R^{c1} and R^{c2} each independently represents a hydrogen atom, an optionally substituted C1-8 alkyl group, an optionally substituted 5- to 10-membered carbocyclic ring, or an optionally substituted 5- to 10-membered heterocyclic ring].

Here, there is no particular limitation for the “substituent” in the “optionally substituted C1-8 alkyl group” represented by R^{c1} and R^{c2} so long as it can be a substituent. Said “substituent” includes, for example, (1) an optionally substituted 5- to 10-membered carbocyclic ring, and (2) an optionally substituted 5- to 10-membered heterocyclic ring. One to five substituent(s) among these optional substituents may be located at any position where substitution is possible.

In the description of the present invention, the “5- to 10-membered carbocyclic ring” in the “optionally substituted 5- to 10-membered carbocyclic ring” includes, for example, a “5- to 10-membered mono- or bi-carbocyclic aryl”. As said “5- to 10-membered mono- or bi-carbocyclic aryl”, there is exemplified a “C5-10 mono- or di-carbocyclic aryl which may be fully or partially saturated”. Said “C5-10 mono- or di-carbocyclic aryl which may be fully or partially saturated” includes, for example, cyclopentane, cyclohexane, cycloheptane, cyclooctane, cyclononane, cyclodecane, cyclopentene, cyclohexene, cycloheptene, cyclooctene, cyclopentadiene, cyclohexadiene, cycloheptadiene, cyclooctadiene, benzene, pentalene, perhydropentalene, azulene, perhydroazulene, indene, perhydroindene, indan, naphthalene, dihydronaphthalene, tetrahydronaphthalene, perhydronaphthalene, and the like. Further, as said “C5-10 mono- or di-carbocyclic aryl which may be fully or partially saturated”, there are exemplified a spiro-bicarbocyclic ring and a bridged bicarbocyclic ring, including, for example, spiro[4.4]nonane, spiro[4.5]decane, bicyclo[2.2.1]heptane, bicyclo[2.2.1]hept-2-ene, bicyclo[3.1.1]heptane, bicyclo[3.1.1]hept-2-ene, bicyclo[2.2.2]octane, bicyclo[2.2.2]oct-2-ene, adamantane, and noradamantane, and the like.

In the description of the present invention, the “5- to 10-membered heterocyclic ring” in the “optionally substituted 5- to 10-membered heterocyclic ring” includes, for example, a “5- to 10-membered mono- or biheterocyclic ring”. Examples of said “5- to 10-membered mono- or bi-heterocyclic ring” are a 5- to 10-membered mono- or bi-cyclic heteroaryl group containing 1 to 5 nitrogen atom(s), 1 to 2 oxygen atom(s) and/or 1 sulfur atom; a 5- to 10-membered heterocyclic ring which is fully or partially saturated; a spiro-bonded biheterocyclic ring; and a bridged biheterocyclic ring. Among 5- to 10-membered mono- or bi-cyclic heteroaryl groups containing 1 to 5 nitrogen atom(s), 1 to 2 oxygen atom(s) and/or one sulfur atom, a heterocyclic ring which is fully or partially saturated, a spiro-bonded biheterocyclic ring, and a bridged biheterocyclic ring, examples of “5- to 10-membered mono- or bi-cyclic heteroaryl group containing 1 to 5 nitrogen atom(s), 1 to 2 oxygen atom(s) and/or 1 sulfur atom” include pyrrole, imidazole, triazole, tetrazole, pyrazole, pyridine, pyrazine, pyrimidine, pyridazine, azepine, diazepine, furan, pyran, oxepine, thiophene, thiopyran, thiepine, oxazole, isoxazole, thiazole, isothiazole, furazan, oxadiazole, oxazine, oxadiazine, oxazepine, oxadiazepine, thiadiazole, thiazine, thiadiazine, thiazepine, thiadiazepine, indole, isoindole, indolidine, benzofuran, isobenzofuran, benzothiophene, isobenzothiophene, indazole, quinoline, isoquinoline, quinolidine, purine, phthalazine, pteridine, naphthyridine, quinoxaline, quinazoline, cinnoline, benzoxazole, benzothiazole, benzimidazole, chromene, benzofurazan, benzothiadiazole, and benzotriazole, and the like.

Further, among “5- to 10-membered mono- or bi-cyclic heteroaryl groups containing 1 to 5 nitrogen atom(s), 1 to 2 oxygen atom(s) and/or 1 sulfur atom”, examples of those which are fully or partially saturated include pyrroline, pyrrolidine, imidazoline, imidazolidine, triazoline, triazolidine, tetrazoline, tetrazolidine, pyrazoline, pyrazolidine, dihydropyridine, tetrahydropyridine, piperidine, dihydropyrazine, tetrahydropyrazine, piperazine, dihydropyrimidine, tetrahydropyrimidine, perhydropyrimidine, dihydropyridazine, tetrahydropyridazine, perhydropyridazine, dihydroazepine, tetrahydroazepine, perhydroazepine, dihydrodiazepine, tetrahydrodiazepine, perhydrodiazepine, dihydrofuran, tetrahydrofuran, dihydropyran, tetrahydropyran, dihydrooxepine, tetrahydrooxepine, perhydrooxepine, dihydrothiophene, tetrahydrothiophene, dihydrothiopyran, tetrahydrothiopyran, dihydrothiepine, tetrahydrothiepine, perhydrothiepine, dihydrooxazole, tetrahydrooxazole(oxazolidine), dihydroisoxazole, tetrahydroisoxazole(isoxazolidine), dihydrothiazole, tetrahydrothiazole(thiazolidine), dihydroisothiazole, tetrahydroisothiazole (isothiazolidine), dihydrofurazan, tetrahydrofurazan, dihydrooxadiazole, tetrahydrooxadiazole(oxadiazolidine), dihydrooxazine, tetrahydrooxazine, dihydrooxadiazine, tetrahydrooxadiazine, dihydrooxazepine, tetrahydrooxazepine, perhydrooxazepine, dihydrooxadiazepine, tetrahydrooxadiazepine, perhydrooxadiazepine, dihydrothiadiazole, tetrahydrothiadiazole(thiadiazolidine), dihydrothiazine, tetrahydrothiazine, dihydrothiadiazine, tetrahydrothiadiazine, dihydrothiazepine, tetrahydrothiazepine, perhydrothiazepine, dihydrothiadiazepine, tetrahydrothiadiazepine, perhydrothiadiazepine, morpholine, thiomorpholine, oxathian, indoline, isoindoline, dihydrobenzofuran, perhydrobenzofurn, dihydroisobenzofuran, perhydroisobenzofuran, dihydrobenzothiophene, perhydrobenzothiophene, dihydroisobenzothiophene, perhydroisobenzothiophene, dihydroindazole, perhydroindazole, dihydroquinoline, tetrahydroquinoline, perhydroquinoline, dihydroisoquinoline, tetrahydroisoquinoline, perhydroisoquinoline, dihydrophthalazine, tetrahydrophthalazine, perhydrophthalazine, dihydronaphthyridine, tetrahydronaphthyridine, perhydronaphthyridine, dihydroquinoxaline, tetrahydroquinoxaline, perhydroquinoxaline, dihydroquinazoline, tetrahydroquinazoline, perhydroquinazoline, dihydrocinnoline, tetrahydrocinnoline, perhydrocinnoline, benzoxathian, dihydrobenzoxazine, dihydrobenzothiazine, pyrazinomorpholine, dihydrobenzoxazole, perhydrobenzoxazole, dihydrobenzothiazole, perhydrobenzothiazole, dihydrobenzimidazole, perhydrobenzimidazole, dioxolane, dioxane, dioxaindane, benzodioxane, andchroman, andthelike. Furthermore, among “5- to 10-membered mono- or bi-cyclic heteroaryl groups containing 1 to 5 nitrogen atom(s), 1 to 2 oxygen atom(s) and/or 1 sulfur atom, a 5- to 10-membered heterocyclic ring which is fully or partially saturated, a spiro-bonded biheterocyclic ring and a bridged biheterocyclic ring”, examples of the spiro-bonded biheterocyclic ring and the bridged biheterocyclic ring include azaspiro[4.4]nonane, oxazaspiro[4.4]nonane, dioxaspiro[4.4]nonane, azaspiro[4.5]decane, thiaspiro[4.5]decane, dithiaspiro[4.5]decane, dioxaspiro[4.5]decane, oxazaspiro[4.5]decane, azabicyclo[2.2.1]heptane, oxabicyclo[2.2.1]heptane, azabicyclo[3.1.1]heptane, azabicyclo[3.2.1]octane, oxabicyclo[3.2.1]octane, azabicyclo[2.2.2]octane, diazabicyclo[2.2.2]octane, and the like.

In the description of the present invention, the “substituent” in the “5- to 10-membered optionally substituted carbocyclic ring” or “5- to 10-membered optionally substituted heterocyclic ring” is not particularly limited so long as it can be a substituent. Said “substituent” includes, for example, (1) a substituent selected from a Fourth Group shown below, and (2) an “optionally substituted 5- to 6-membered cyclic group” shown below. Here, one to five substituent(s) among these optional substituents may be located at any position where substitution is possible.

<Fourth Group>

(a) C1-8 alkyl, (b) halogen atom, (c) nitro, (d) cyano, (e) —OR^{d1}, (f) —NR^{d1}R^{d2}, (g) —COOR^{d1}, (h) —COR^{d1}, (i) —CONR^{d1}R^{d2}, (i) —NR^{d1}COR^{d2}, (k) —SO₂NR^{d1}R^{d2}, (l) —NR^{d1}SOR^{d2}, (m) —SR^{d1}, (n) —SO₂R^{d1}, (o) oxo, and (p) thioxo [in these groups, R^{d1} and R^{d2} each independently represents a hydrogen atom or a C1-8 alkyl group].

In the description of the present invention, as the “5- to 6-membered cyclic group” in the “optionally substituted 5- to 6-membered cyclic group”, there are exemplified a “5- to 6-membered monocarbocyclic ring” and a “5- to 6-membered monoheterocyclic ring”. Said “5- to 6-membered monocarbocyclic ring” includes, for example, a “C5-6 monocarbocyclic aryl group which may be fully or partially saturated”. Examples of said “C5-6 monocarbocyclic aryl group which may be fully or partially saturated” are cyclopentane, cyclohexane, cyclopentene, cyclohexene, cyclopentadiene, cyclohexadiene, benzene and the like. On the other hand, as the “5- to 6-membered monoheterocyclic ring”, there are exemplified a “5- to 6-membered monoheteroaryl group containing 1 to 4 nitrogen atom(s), 1 to 2 oxygen atom(s) and/or 1 sulfur atom which may be fully or partially substituted”.

Among those “5- to 6-membered monoheteroaryl groups containing 1 to 4 nitrogen atom(s), 1 to 2 oxygen atom(s) and/or 1 sulfur atom which may be fully or partially substituted”, examples of the “5- to 6-membered monoheteroaryl group containing 1 to 4 nitrogen atom(s), 1 to 2 oxygen atom(s) and/or 1 sulfur atom” includes pyrrole, imidazole, triazole, tetrazole, pyrazole, pyridine, pyrazine, pyrimidine, pyridazine, furan, pyran, thiophene, thiopyran, oxazole, isoxazole, thiazole, isothiazole, furazan, oxadiazole, oxazine, oxadiazine, thiadiazole, thiazine, thiadiazine and the like. Further, among those “5- to 6-membered monoheteroaryl groups containing 1 to 4 nitrogen atom(s), 1 to 2 oxygen atom(s) and/or 1 sulfur atom”, there are exemplified those which are fully or partially saturated, including, for example, pyrroline, pyrrolidine, imidazoline, imidazolidine, triazoline, triazolidine, tetrrazoline, tetrazolidine, pyrazoline, pyrazolidine, dihydropyridine, tetrahydropyridine, piperidine, dihydropyrazine, tetrahydropyrazine, piperazine, dihydropyrimidine, tetrahydropyrimidine, perhydropyrimidine, dihydropyridazine, tetrahydropyridazine, perhydropyridazine, dihydrofuran, tetrahydrofuran, dihydropyran, tetrahydropyran, dihydrothiophene, tetrahydrothiophene, dihydrothiopyran, tetrahydrothiopyran, dihydrooxazole, tetrahydrooxazole(oxazolidine), dihydroisoxazole, tetrahydroisoxazole(isoxazolidine), dihydrothiazole, tetrahydrothiazole(thiazolidine), dihydroisothiazole, tetrahydroisothiazole(isothiazolidine), dihydrofurazan, tetrahydrofurazan, dihydrooxadiazole, tetrahydrooxadiazole(oxadiazolidine), dihydrooxazine, tetrahydrooxazine, dihydrooxadiazine, tetrahydrooxadiazine, dihydrothiadiazole, tetrahydrothiadiazole(thiadiazolidine), dihydrothiazine, tetrahydrothiazine, dihydrothiadiazine, tetrahydrothiadiazine, morpholine, thiomorpholine, oxathian, dioxolane, and dioxane, and the like.

The “5- to 6-membered cyclic group” in said “5- to 6-membered optionally substituted cyclic group” can be preferably, for example, a “5- to 6-membered monocarbocyclic ring” or a “5- to 6-membered monoheterocyclic ring”, more preferably benzene, pyrazole, and the like.

There is no particular limitation for the “substituent” in said “5- to 6-membered optionally substituted cyclic group” as far as it can be a substituent. Examples of said “substituent” include, for example, (1) C1-8 alkyl, (2) C1-8 alkoxy (e.g. methoxy, ethoxy, propoxy, isopropoxy, butoxy, sec-butoxy, t-butoxy, pentyloxy, hexyloxy, heptyloxy, octyloxy, and isomers thereof), (3) halogen atom, (4) trifluoromethyl, (5) trifluoromethoxy, and 1 to 5 of those substituents may be located at any position where substitution is possible.

In the description of the present invention, as the “cyclic group” in the “optionally substituted cyclic group” represented by A or ring B, there are exemplified a carbocyclic ring and a heterocyclic ring. The “carbocyclic ring” includes, for example, the “5- to 10-membered carbocyclic ring” as described above, and the “heterocyclic ring” includes, for example, the “5- to 10-membered heterocyclic ring” as described above.

In the description of the present invention, there is no particular limitation for the “substituent” in the “optionally substituted cyclic group” so long as it can be a substituent. Examples of the substituents are (1) the “optionally substituted aliphatic hydrocarbon group” as described above, (2) a substituent selected from the First Group as mentioned above, (3) the “5- to 10-membered optionally substituted carbocyclic group” as described above, or (4) the “5- to 10-membered optionally substituted heterocyclic group”, wherein 1 to 12, preferably 1 to 3 of those substituents may be located at any position where substitution is possible.

In the description of the present invention, an “optionally protected amino group” represented by the symbols A is an amino group wherein 1 or 2 hydrogen atoms may be substituted by an optional substituent, and said “substituent” includes, for example, (1) the “optionally substituted cyclic group” as described above and (2) the “optionally substituted aliphatic hydrocarbon group” as described above.

In the description of the present invention, a “spacer having 1 to 4 atom(s) in the main chain” represented by E means a space formed by connecting 1 to 4 atom(s) in the main chain. Here, the number of atoms in the main chain should be counted as such that it must be the minimum. For example, the number of atoms in 1,2-cyclopentylene is 2, and that of in 1,3-cyclopentylene is 3.

As the “spacer having 1 to 4 atoms in the main chain”, there are exemplified an optionally substituted C1-4 alkylene group (e.g. —CH2-, —(CH₂)₂—, —(CH₂)₃—, —(CH₂)₄—, etc.), an optionally substituted C2-4 alkenylene group (e.g. —CH═CH—, —CH₂—CH═CH—, —CH═CH—CH₂—, —(CH₂)₂—CH═CH—, —CH═CH—(CH₂)₂—, —CH₂—CH═CH—CH₂—, etc.), and an optionally substituted C2-4 alkynylene group (e.g. —C≡C—, —CH₂—C≡C—, —C≡C—CH₂—, —(CH₂)₂—C≡C—, —C≡C—(CH₂)₂—, —CH₂—C≡C—CH₂—, etc.). Further, any carbon atom in the C1-4 alkylene, C2-4 alkenylene, and C2-4 alkynylene group may be replaced by oxygen, sulfur, or optionally substituted nitrogen atom. Here, there is no particular limitation for the “substituent” in the “optionally substituted nitrogen atom so long as it can be a substituent. Said “substituent” includes, for example, an optionally substituted C1-8 alkyl group. The “substituent” in the “optionally substituted C1-8 alkyl group” is not particularly limited so long as it can be a substituent. Examples of the “substituent” are (1) hydroxy, and (2) the above “optionally substituted C5-6 cyclic group”, and these substituents may be located at any position where substitution is possible.

There is no particular limitation for the “spacer having 1 to 4 atom(s) in the main chain” represented by E such as “optionally substituted C1-4 alkylene”, “optionally substituted C2-4 alkenylene” and “optionally substituted C2-4 alkynylene”, so long as it can be a substituent. Examples of such substituents are (1) C1-8 alkyl, (2) C1-8 alkoxy, (3) halogen, (4) hydroxy, (5) oxo, (6) thioxo, (7) amino, (8) ═N—ORⁿ [in these groups, Rⁿ is a hydrogen atom or the same as defined in the “substituent” of the “optionally substituted nitrogen atom” as defined above. One to five substituent(s) among these optional substituents may be located at any position where substitution is possible.

In the description of the present invention, when Z is a bond, it means that A is directly bonded to K.

In the description of the present invention, the “optionally substituted cyclic group” represented by R^{z} and R^{zz} has the same meaning as the “optionally substituted cyclic group” represented by A described above.

In the present invention, the “optionally substituted aliphatic hydrocarbon group” represented by R^{z} and R^{zz} has the same meaning as the “optionally substituted aliphatic hydrocarbon group” represented by A described above.

In the description of the present invention, the “substituent” in the “optionally substituted methylene group” represented by —C(=T) has the same meaning as the “optionally substituted C1-8 alkyl group” described above.

In the description of the present invention, with respect to the “heterocyclic ring containing at least one nitrogen atom” in the “optionally further substituted heterocyclic ring containing at least one nitrogen atom” represented by ring D, there is no limitation on the ring-constituting atoms, so long as one of the ring-constituting atoms is a nitrogen atom bonded to E described above, another one is a “carbon atom to which =T group is bonded”, and a further another one is K (i.e. nitrogen atom or carbon atom) adjacent to the “carbon atom to which =T group is bonded”. The “heterocyclic ring containing at least one nitrogen atom” includes, for example, a “5- to 10-membered monoheterocyclic ring containing at least one nitrogen atom”. When K represents a nitrogen atom, examples of the “5- to 10-membered monoheterocyclic ring containing at least one nitrogen atom” are imidazolidine, triazoline, triazolidine, tetrazoline, tetrazolidine, pyrazolidine, piperazine, tetrahydropyrimidine, perhydropyrimidine, perhydropyridazine, tetrahydrodiazepine, perhydrodiazepine, tetrahydrofurazan, tetrahydrooxadiazole(oxadiazolidine), tetrahydrooxadiazine, perhydrooxadiazepine, tetrahydrothiadiazole(thiadiazolidine), tetrahydrothiadiazine, perhydrothiadiazepine, and the like.

Further, when K represents a carbon atom, examples of the “5- to 10-membered monoheterocyclic ring containing at least one nitrogen atom” are pyrroline, pyrrolidine, imidazoline, imidazolidine, pyrazoline, pyrazolidine, dihydropyridine, tetrahydropyridine, piperidine, dihydropyrazine, tetrahydropyrazine, piperazine, dihydropyrimidine, tetrahydropyrimidine, perhydropyrimidine, dihydropyridazine, tetrahydropyridazine, perhydropyridazine, dihydroazepine, tetrahydroazepine, perhydroazepine, dihydrodiazepine, tetrahydrodiazepine, perhydrodiazepine, tetrahydrooxazole(oxazolidine), tetrahydroisoxazole(isooxazolidine), tetrahydrothiazole(thiazolidine), tetrahydroisothiazole(isothiazolidine), dihydrofurazan, tetrahydrofurazan, tetrahydrooxazine, perhydrooxazepine, tetrahydrooxazepine, perhydrooxadiazepine, tetrahydrothiazine, perhydrothiazepine, tetrahydrothiadiazepine, perhydrothiadiazepine, morpholine, thiomorpholine, and the like.

In the description of the present invention, there is no limitation for the “substituent” in the “heterocyclic ring further containing at least one nitrogen atom” represented by ring D so long as it can be a substituent. Examples of the “substituent” are (1) optionally substituted C1-8 alkyl, (2) the “optionally substituted 5- to 6-membered cyclic group” as defined above, (3) a substituent selected from a Fifth Group shown below, (4) oxo, and 1 to 9 substituent(s) of these optional substituents, preferably 1 to 3 substituent(s) may be located at any position where substitution is possible. Here, there is no particular limitation for the substituent in the “optionally substituted C1-8 alkyl group” as a substituent on the ring D, so long as it can be a substituent. Examples of such substituents are (1) the above “optionally substituted 5- to 6-membered cyclic group” and (2) a substituent selected from a Fifth Group shown below, and 1 to 5 substituent(s) among these substituents may be optionally located at any position where substitution is possible.

<Fifth Group>

(a) —OR^{e1}, (b) —NR^{e1}R^{e2}, (c) —COOR^{e1}, (d) —CONR^{e1}R^{e2}, (e) —NR^{e1}COR^{e2}, (f) —SO₂R^{e1}, (g) —SO₂NR^{e1}R^{e2}, (h) —NR^{e1}SO₂R^{e2} and (i) —NR^{e1}COOR^{e2} [wherein R^{e1} and R^{e2} each independently represents a hydrogen atom or an optionally substituted C1-8 alkyl group]

Here, there is no particular limitation for the “substituent” in the “optionally substituted C1-8 alkyl group”, so long as it can be a substituent. The “substituent” includes, for example, the “optionally substituted 5- to 6-membered cyclic group” as defined above, and 1 to 5 substituent (s) among these substituents may be optionally located at any position where substitution is possible.

In the description of the present invention, the “optionally further substituted 6-membered heterocyclic ring containing at least one nitrogen atom” represented by the ring D means a 6-membered ring selected from the hereinbefore mentioned “optionally further substituted heterocyclic ring containing at least one nitrogen atom”. For example, there are exemplified an optionally further substituted tetrahydropyrimidine, an optionally further substituted perhydropyrimidine, an optionally further substituted piperidine and an optionally further substituted piperazine.

In the description of the present invention, the “optionally substituted 5- to 10-membered cyclic group” includes, for example, the “optionally substituted 5- to 10-membered carbocyclic ring” as defined above, and the “optionally substituted 5- to 10-membered heterocyclic ring” as defined above.

In the present invention, any rings, groups and atoms represented by A; ring B; E; K; T; Z; ring D; R^{z}; and R^{zz} are all preferable. Especially, embodiments as described in Examples are preferred. Hereinafter, preferable groups, preferable rings and preferable atoms are listed, and all symbols as used herein have the same meanings as those defined above.

In the description of the present invention, examples of A include, for example, preferably “an optionally substituted cyclic group” and “an optionally substituted aliphatic hydrocarbon group”, and more preferably “an optionally substituted aliphatic hydrocarbon group”. The “aliphatic hydrocarbon group” in the “optionally substituted aliphatic hydrocarbon group” includes, for example, preferably “a C1-8 alkyl group”, more preferably methyl. Further, examples of the “substituent” in the “optionally substituted aliphatic hydrocarbon group” are “an optionally substituted 5- to 10-membered carbocyclic ring”; C1-8 alkyl; halogen; —NR^{a1}R^{a2}; —NR^{a1}COR^{a2}; —COOR^{a2}; —CONR^{a1}R^{a2}; —COR^{a1}; —SO₂NR^{a1}R^{a2}; —NR^{a1}SO₂R^{a2}; C1-4 alkyl substituted by —OR^{a1}; or oxo; more preferably, optionally substituted phenyl, C1-4 alkyl, halogen, —CONR^{a1}R^{a2}, —NR^{a1}R^{a2}, —NR^{a1}COR^{a2}, or oxo, among which an optionally substituted phenyl or oxo is especially preferable.

In the present invention, the “cyclic group” in the “optionally cyclic group” represented by ring B includes, for example, preferably a “5- to 10-membered mono- or bi-carbocyclic ring”, especially preferably benzene and naphthalene. The “substituent” in the “optionally substituted cyclic group” includes, for example, preferably C1-8 alkyl; halogen; —OR^{a1}; —NR^{a1}COR^{a2}; —CONR^{a1}R^{a2}; —NR^{a1}COOR^{a2}; —NR^{a1}, CONR^{a2}R^{a3}; —OCONR^{a1}R^{a2}; C1-8 alkyl substituted by —NR^{a1}COR^{a2}; C1-8 alkyl substituted by —CONR^{a1}R^{a2}; C1-8 alkyl substituted by —NR^{a1}COOR^{a2}; C1-8 alkyl substituted by —NR^{a1}CONR^{a2}R^{a3}; C1-8 alkyl substituted by —OCONR^{a1}R^{a2}; more preferably C1-4 alkyl; halogen; —NR^{a1}CONR^{a2}R^{a3}; —OCONR^{a1}R^{a2}; C1-4 alkyl substituted by —NR^{a1}CONR^{a2}R^{a3}; C1-4 alkyl substituted by —OCONR^{a1}R^{a2}; and C1-4 alkyl substituted by —CONR^{a1}R^{a2}; especially preferably methyl, fluoro, chloro, —NR^{a1}CONR^{a2}R^{a3}, —OCONR^{a1}R^{a2}, —CH₂—CONR^{a1}R^{a2}, —CH₂—NR^{a1}CONR^{a2}R^{a3}, and —CH₂—OCONR^{a1}R^{a2}, and the like.

In the present invention, the “spacer having 1 to 4 atom(s)” in its main chain represented by E includes, for example, preferably C1-4 alkylene; C1-4 alkylene substituted by hydroxy; C1-4 alkylene substituted by oxo; C1-4 alkylene substituted by C1-4 alkyl; C1-4 alkylene substituted by C1-4 alkoxy; —S—; and —C(═NORⁿ), especially preferably, for example, methylene, hydroxymethylene, methoxymethylene, hydroxyiminomethylene, methylmethylene, and carbonyl, and the like.

The “heterocyclic ring containing at least one nitrogen atom” in the “optionally substituted further containing at least one nitrogen atom” represented by ring D include, for example, a “5- to 7-membered heterocyclic ring containing at least one nitrogen atom”, more preferably, for example, a “6-membered heterocyclic ring containing at least one nitrogen atom”, especially preferably, for example, tetrahydropyrimidinone, perhydropyrimidinone, piperidine and piperazine.

In the present invention, 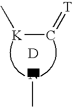
wherein all symbols have the same meanings as defined above, is preferably 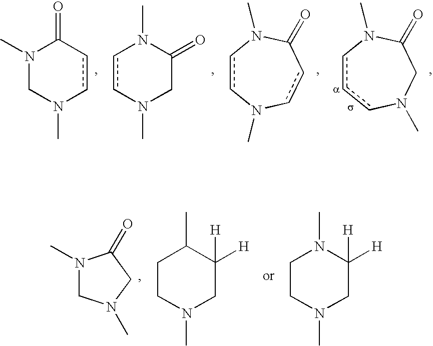
wherein all symbols have the same meanings as defined above, more preferably

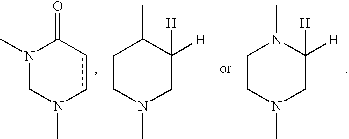

In the present invention, the “substituent” in the “optionally further substituted heterocyclic ring containing at least one nitrogen atom” includes, for example, oxo; C1-8 alkyl; —OR^{e1}; —COOR^{e1}; and C1-4 alkyl substituted by an optionally substituted 5- to 6-membered cyclic group, more preferably oxo, C1-4 alkyl, —OR^{e1} and —COOR^{e1}, and especially preferably, oxo, methyl, ethyl, —OH, —OCH₃, —COOH and —COOCH₃.

In the present invention, K is preferably “a nitrogen atom”.

In the present invention, —C(=T) is preferably —C(═O) or an optionally substituted methylene group, more preferably an optionally substituted methylene group.

In the present invention, Z is preferably “an oxygen atom or a bond”, more preferably a bond.

In the present invention, a compound represented by formula (I) comprising a combination of preferable groups, preferable rings, and preferable atoms as defined above is preferable, and it includes, for example, especially preferably a compound represented by formula (I-2): 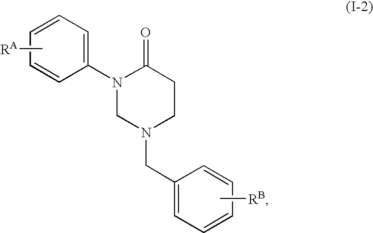
wherein R^{A} and R^{B} each represents the “substituent” in the “optionally substituted cyclic group” as defined above;

a compound represented by formula (I-3): 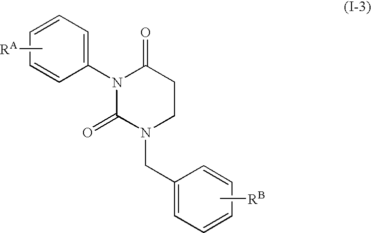
wherein all symbols have the same meanings as defined above;

a compound represented by formula (I-4): 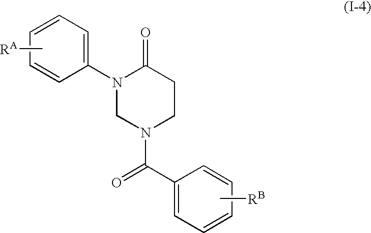
wherein all symbols have the same meanings as defined above;

a compound represented by formula (I-5): 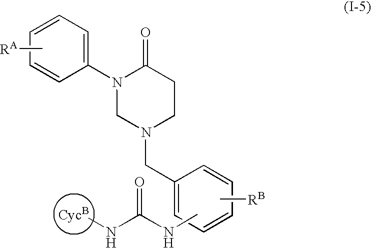
wherein Cyc^{B} represents the “optionally substituted 5- to 10-membered heterocyclic ring” as defined above, and other symbols have the same meanings as defined above;

a compound represented by formula (I-6): 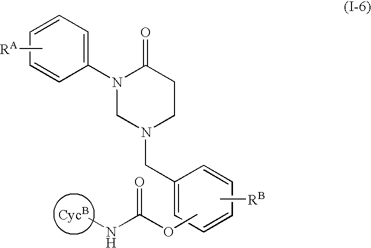
wherein all symbols have the same meanings as defined above;

a compound represented by formula (I-7): 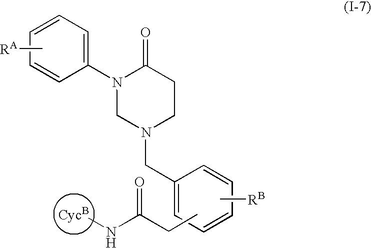
wherein all symbols have the same meanings as defined above;

a compound represented by formula (I-8): 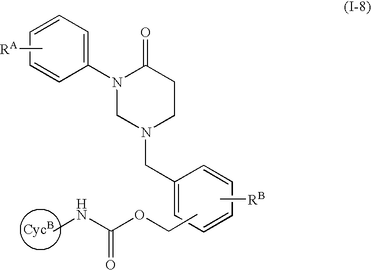
wherein all symbols have the same meanings as defined above;

a compound represented by formula (I-9): 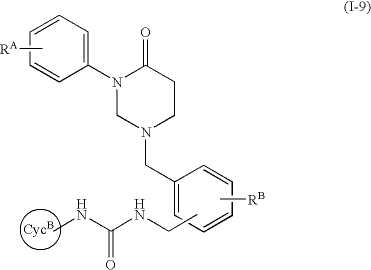
wherein all symbols have the same meanings as defined above;

a compound represented by formula (I-10): 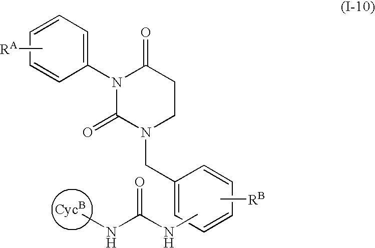
wherein all symbols have the same meanings as defined above;

a compound represented by formula (I-11): 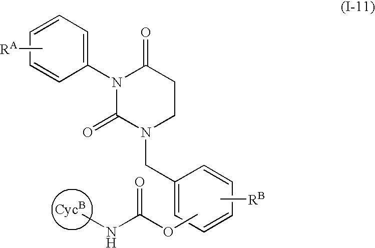
wherein all symbols have the same meanings as defined above;

a compound represented by formula (I-12): 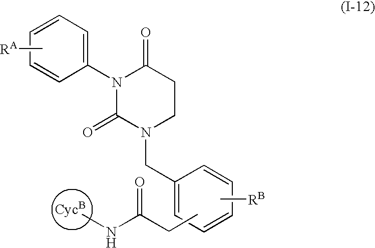
wherein all symbols have the same meanings as defined above;

a compound represented by formula (I-13): 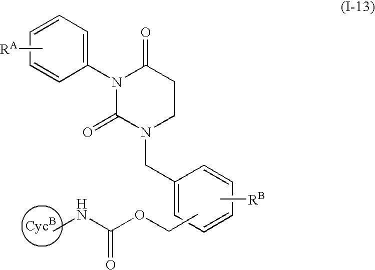
wherein all symbols have the same meanings as defined above;

a compound represented by formula (I-14): 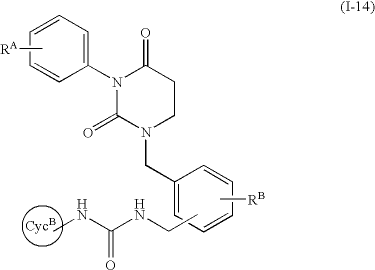
wherein all symbols have the same meanings as defined above;

a compound represented by formula (I-15): 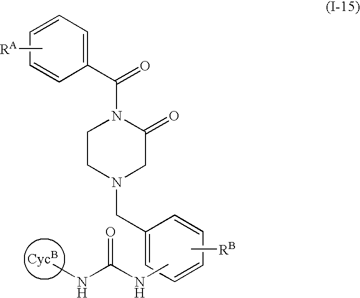
wherein all symbols have the same meanings as defined above;

a compound represented by formula (I-16): 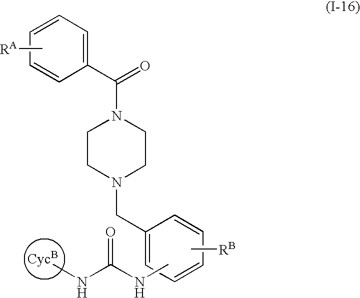
wherein all symbols have the same meanings as defined above; and

a compound represented by formula (I-17): 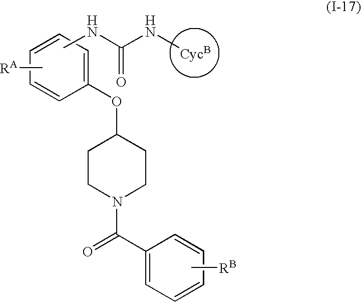
wherein all symbols have the same meanings as defined above. In the above formulae (I-2) to (I-14), compounds wherein 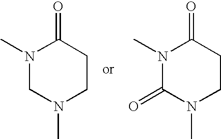
corresponding to the ring D is replaced by 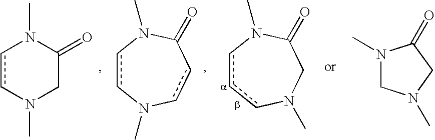
wherein all symbols have the same meanings as defined above, are preferable.

Here, “5- to 10-membered heterocyclic ring” in the “optionally substituted 5- to 10-membered heterocyclic ring” represented by Cyc^{B} includes, for example, a “5- to 6-membered monoheterocyclic ring” and especially preferably a pyrazole ring. The “substituent” in the “optionally substituted 5- to 10-membered heterocyclic ring” represented by Cyc^{B} includes, for example, preferably a C1-8 alkyl group and an optionally substituted 5- to 6-membered cyclic group, especially preferably methyl, t-butyl, 4-methylphenyl, and the like.

In the present invention, all the compounds described in Examples are preferred. Further, the compounds 1) to 25) hereinafter described are also all preferred.
1) N-[3-t-butyl-1-(4-methylphenyl)-1H-pyrazol-5-yl]-N′-(3-{[4-(2-methylbenzoyl)-1-piperazinyl]methyl}phenyl)urea,
2) N-[3-t-butyl-1-(4-methylphenyl)-1H-pyrazol-5-yl]-N′-(2-methyl-4-{[4-(2-methylbenzoyl)-1-piperazinyl]methyl}phenyl)urea,
3) N-[3-t-butyl-1-(4-methylphenyl)-1H-pyrazol-5-yl]-N′-(3-chloro-4-{[4-(2-methylbenzoyl)-1-piperazinyl]methyl}phenyl)urea,
4) N-[3-t-butyl-1-(4-methylphenyl)-1H-pyrazol-5-yl]-N′-[4-({4-[3-(dimethylamino)benzoyl]piperazin-1-yl}methyl)-2-methylphenyl]urea,
5) N-[3-t-butyl-1-(4-methylphenyl)-1H-pyrazol-5-yl]-N′-{4-[(4-{4-[(E)-(hydroxyimino)methyl]benzoyl}piperazin-1-yl)methyl]-2-methylphenyl}urea,
6) N-[3-t-butyl-1-(4-methylphenyl)-1H-pyrazol-5-yl]-N′-{4-[(4-{4-[(dimethylamino)methyl]benzoyl}piperazin-1-yl)methyl]-2-methylphenyl}urea,
7) N-[3-t-butyl-1-(4-methylphenyl)-1H-pyrazol-5-yl]-N′-(2,6-dimethyl-4-{[4-(2-methylbenzoyl)piperazin-1-yl]methyl}phenyl)urea,
8) N-[3-t-butyl-1-(4-methylphenyl)-1H-pyrazol-5-yl]-N′-(2-fluoro-4-{[4-(2-methylbenzoyl)piperazin-1-yl]methyl}phenyl)urea,
9) N-[3-t-butyl-1-(4-methylphenyl)-1H-pyrazol-5-yl]-N′-{2-methyl-4-[(4-{4-[(4-methylpiperazin-1-yl)methyl]benzoyl}piperazin-1-yl)methyl]phenyl}urea,
10) N-[3-t-butyl-1-(4-methylphenyl)-1H-pyrazol-5-yl]-N′-(2-methyl-5-{[4-(2-methylbenzoyl)piperazin-1-yl]methyl}phenyl)urea,
11) N-{4-[(1-benzoylpiperidin-4-yl)oxy]-2-methylphenyl}-N′-[3-t-butyl-1-(4-methylphenyl)-1H-pyrazol-5-yl]urea,
12) N-[3-t-butyl-1-(4-methylphenyl)-1H-pyrazol-5-yl]-N′-{4-[(4-{4-[(dimethylamino)methyl]benzoyl}piperazin-1-yl)methyl]-2-fluorophenyl}urea,
13) N-[3-t-butyl-1-(4-methylphenyl)-1H-pyrazol-5-yl]-N′-{2-fluoro-4-[(4-{4-[(4-methylpiperazin-1-yl)methyl]benzoyl}piperazin-1-yl)methyl]phenyl}urea,
14) N-[3-t-butyl-1-(4-methylphenyl)-1H-pyrazol-5-yl]-N′-{3-[(4-{4-[(dimethylamino)methyl]benzoyl}piperazin-1-yl)methyl]-2-methylphenyl}urea,
15) N-[3-t-butyl-1-(4-methylphenyl)-1H-pyrazol-5-yl]-N′-{2-methyl-3-[(4-{4-[(4-methylpiperazin-1-yl)methyl]benzoyl}piperazin-1-yl)methyl]phenyl}urea,
16) N-[3-t-butyl-1-(4-methylphenyl)-1H-pyrazol-5-yl]-N′-{5-[(4-{4-[(dimethylamino)methyl]benzoyl}piperazin-1-yl)methyl]-2-methylphenyl}urea,
17) N-[3-t-butyl-1-(4-methylphenyl)-1H-pyrazol-5-yl]-N′-{2-methyl-5-[(4-{4-[(4-methylpiperazin-1-yl)methyl]benzoyl}piperazin-1-yl)methyl]phenyl}urea,
18) N-[3-t-butyl-1-(4-methylphenyl)-1H-pyrazol-5-yl]-N′-(2-fluoro-5-{[4-(2-methylbenzoyl)piperazin-1-yl]methyl}phenyl)urea,
19) N-[3-t-butyl-1-(4-methylphenyl)-1H-pyrazol-5-yl]-N′-(2-chloro-5-{[4-(2-methylbenzoyl)piperazin-1-yl]methyl}phenyl)urea,
20) N-[3-t-butyl-1-(4-methylphenyl)-1H-pyrazol-5-yl]-N′-2-methoxy-5-{[4-(2-methylbenzoyl)piperazin-1-yl]methyl}phenyl)urea,
21) N-[3-t-butyl-1-(4-methylphenyl)-1H-pyrazol-5-yl]-N′-{2-methyl-4-[(1-{4-[(4-methylpiperazin-1-yl)methyl]benzoyl}piperidin-4-yl)oxy]phenyl}urea,
22) N-[3-t-butyl-1-(4-methylphenyl)-1H-pyrazol-5-yl]-N′-{2-fluoro-5-[(4-{4-[(4-methylpiperazin-1-yl)methyl]benzoyl}piperazin-1-yl)methyl]phenyl}urea,
23) N-(3-t-butyl-1-(4-methylphenyl)-1H-pyrazol-5-yl]-N′-{2-methyl-4-[(4-{3-[(4-methylpiperazin-1-yl)methyl]benzoyl}piperazin-1-yl)methyl]phenyl}urea,
24) N-[3-t-butyl-1-(4-methylphenyl)-1H-pyrazol-5-yl]-N′-{2-chloro-5-[(4-{4-[(4-methylpiperazin-1-yl)methyl]benzoyl}piperazin-1-yl)methyl]phenyl}urea, and
25) N-[3-t-butyl-1-(4-methylphenyl)-1H-pyrazol-5-yl]-N′-{2-methyl-4-[(4-{2-[(4-methylpiperazin-1-yl)methyl]benzoyl}piperazin-1-yl)methyl]phenyl}urea.

Unless otherwise specified, any isomers are all included in the present invention. For example, linear or branched ones are included in the alkyl, alkoxy, and alkylene groups. Further, the present invention includes isomers due to double bond, ring, and fused ring (E-form, Z-form, cis-form, trans-form), isomers due to the presence of asymmetric carbon atom (R-form, S-form, α-form, β-form, enantiomer, diastereomer), optically active compounds with optical rotation (D-form, L-form, d-form, l-form), polar compounds obtained by chromatographic separation (high polar compound, low polar compound), equilibrium compounds, and mixtures of these compounds in an arbitrary ratio, and racemates. Moreover, the present invention includes all tautomers.

[Salts, N-Oxides, Solvates and Prodrugs]

Pharmacologically acceptable salts are all included in the salts of compounds represented by formula (I). The pharmacologically acceptable salts are preferably those which are non-toxic and soluble in water. Examples of suitable salts are salts of alkali metals (e.g. potassium, sodium, lithium, etc.), salts of alkaline earth metals (e.g. calcium, magnesium, etc.), ammonium salts (e.g. tetramethylammonium salt, tetrabutylammonium salt, etc.), salts of organic amines (e.g. triethylamine, methylamine, dimethylamine, cyclopentylamine, benzylamine, phenethylamine, piperidine, monoethanolamine, diethanolamine, tris(hydroxymethyl)methylamine, lysine, arginine, N-methyl-D-glucamine, etc.), acid addition salts [inorganic acid salts (e.g. hydrochloride, hydrobromide, hydroiodide, sulfate, phosphate, nitrate, etc.), organic acid salts (e.g. acetate, trifluoroacetate, lactate, tartarate, oxalate, fumarate, maleate, benzoate, citrate, methanesulfonate, ethanesulfonate, benzenesulfonate, toluenesulfonate, isethionate, glucronate, gluconate, etc.), etc.].

Further, such salts include quaternary ammonium salts. The quaternary ammonium salts can be those wherein the nitrogen atom in the compound represented by formula (I) is quaternized by R⁰ group. Examples of R⁰ are a C1-8 alkyl group, and a phenyl-substituted C1-8 alkyl group.

The N-oxides of compounds represented by the formula (I) are ones wherein the nitrogen atom of the compound represented by formula (I) is oxidized. Also, the N-oxides of the present invention may be present in the form of alkaline (earth) metal salts, ammonium salts, organic amine salts or acid addition salts.

Suitable solvates of compounds represented by formula (I) includes, for example, a solvate with water or an alcoholic solvent (e.g. ethanol, etc.). The solvates are preferably non-toxic and soluble in water. The solvates of the present invention includes solvates of alkaline (earth) metal salts, ammnoium salts, organic amine salts, acid addition salts or N-oxides of the inventive compounds represented by formula (I).

The compounds of the present invention may be converted into the above salts, the above N-oxides, or the above solvates by the known method.

The prodrugs of the compounds represented by formula (I) are those which can be converted into the compounds of the formula (I) of the present invention by the in vivo action of enzymes or gastric acid.

Examples of the prodrugs of compounds represented by formula (I) are (1) those wherein the amino group is acylated, alkylated, or phosphorylated (for example, compounds wherein the amino group is eicosanoylated, alanylated, pentylaminocarbonylated, (5-methyl-2-oxo-1,3-dioxolen-4-yl)methoxycarbonylated, tetrahydrofuranylated, pyrrolidylmethylated, pivaloyloxymethylated, acetoxymethylated, t-butylated, etc.), when compounds represented by formula (I) contain an amino group, (2) those wherein the hydroxy group is acylated, alkylated, phosphorylated, or borated (for example, compounds wherein the hydroxy group is acetylated, palmitoylated, propanoylated, pivaloylated, succinylated, fumarylated, alanylated, dimethylaminocabonylated, etc.), when compounds represented by formula (I) contain a hydroxy group, and (3) those wherein the carboxyl group is esterified, or amidated (for example, compounds wherein the carboxyl group is converted into an ester such as ethyl ester, phenyl ester, carboxymethyl ester, dimethylaminomethyl ester, pivaloyloxymethyl ester, ethoxycarbonyloxyethyl ester, phthalidyl ester, (5-methyl-2-oxo-1,3-dioxolen-4-yl)methyl ester, and cyclohexyloxycarbonylethyl ester, or compounds which are methylamidated). These compounds can be prepared by the conventional method. The prodrug of the compound represented by formula (I) is any one of hydrates and non-hydrates. The prodrug of the compound represented by formula (I) is a compound which may be converted into a compound represented by formula (I) under physiological conditions as described in Development of Drugs, Molecule Design, vol. 7, pp. 163-198, (1990), published by Hirokawa Publishing Co., Japan. Further, the compound represented by formula (I) may be labelled with an isotope (e.g. ³H, ¹⁴C, ³⁵S, ¹²⁵ I, etc.).

Mechanical IUPAC nomenclature of the compounds represented by formula (I) was performed using a computer program ACD/NAME™ available from Advanced Chemistry Development Co. For example, the following compound was named 3-(4-bromo-2,6-dichlorophenyl)-1-(2,4-difluorobenzyl)tetrahydropyrimidin-4(1H)-one.

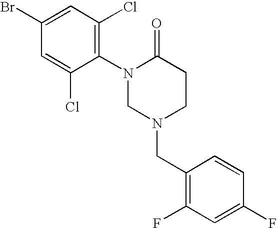

### PRODUCTION OF COMPOUNDS OF THE PRESENT INVENTION

The compounds represented by formula (I) can be prepared by the known method, for example, an appropriately improved or combined method of Methods (A) to (F) shown below, similar methods thereof, the method as described in Examples, and the method described in Comprehensive Organic Transformations: A Guide to Functional Group Preparations, 2nd Edition (Richard C. Larock, John Wiley & Sons Inc., 1999).

The starting material in each production method shown below may be used in the form of a salt. Such salt used is the salt of compounds represented by formula (I) as defined above.

(A) A compound wherein —C(=T)- is —C(═O)— or an optionally substituted methylene group, and any other substituents do not contain a thioxo group can be prepared by reacting a compound represented by formula (II): 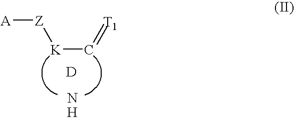
wherein —C(=T1)- is —C(═O) or an optionally substituted methylene group, and other symbols have the same meanings as defined above, with a compound represented by formula (III-1):
X-E [symbol] (III -1)
wherein X is a leaving group (e.g. chlorine, bromine, iodine, p-toluenesulfonyloxy, methanesulfonyloxy, trifluoromethanesulfonyloxy, etc.) and other symbols have the same meanings as defined above.

This reaction is known, and the desired compounds can be prepared, for example, by:
1) alkylation when E is an alkylene group,
2) amidation when E is a carbonyl-containing group, or
3) sulfonamidation when E is a sulfonyl-containing group.

In addition to the above reaction, for example, a bond formation between a nitrogen atom and an E group can be performed according to the method described in “Comprehensive Organic Transformations: A Guide to Functional Group Preparations”, 2nd Edition, John Wiley & Sons, 1999.

Since those skilled in the art easily understand the reaction, the desired compounds of the present invention can be easily prepared by selectively using the reaction depending on the kind of each E group.

1) Alkylation reaction is known per se, and for example, an amine is reacted with a compound containing a leaving group at −78° C. to 100° C. in an organic solvent (e.g. aromatic hydrocarbons such as benzene, toluene, and xylene; halogenated hydrocarbons such as dichloromethane and chloroform; saturated hydrocarbons such as hexane, heptane, and cyclohexane; ethers such as diethyl ether, tetrahydrofuran, and 1,4-dioxane; ketones such as acetone and methyl ethyl keone; nitriles such as acetonitrile; sulfoxides such as dimethyl sulfoxide; acid amides such as N,N-dimethylformamide; or esters such as ethyl acetate. These solvents may be used alone, or if required, in combination of two or more solvents in an appropriate mixing ratio of, for example, 1:1 to 1:10) in the presense of a base (for example, inorganic bases including alkali or alkaline earth metal hydrides such as sodium hydride and potassium hydride; alkyl lithiums such as butyl lithium, sec-butyl lithium, and t-butyl lithium; alkali metal alkoxides such as sodium methoxide and sodium ethoxide; alkal metals such as metallic sodium and metallic potassium; organic bases including alkylamines such as triethylamine, tributylamine and diisopropylethylamine; aromatic amines such as N,N-dimethylaniline, pyridine, lutidine, collidine, 4-(dimethylamino)pyridine; and DBU (1,8-diazabicyclo[5.4.0]undecene-7); metal amides such as lithium diisopropylamide, lithium hexamethyldisilazide, calcium hexamethyldisilazide, and sodium hexamethyldisilazide).

2) Amidation reaction is known per se, and it includes, for example,

(1) a method using an acid halide,

(2) a method using a mixed anhydride, and

(3) a method using a condensing agent.

Details about these methods are hereinafter illustrated.

(1) As for the method using an acid halide, a carboxylic acid is reacted with an acid halide (for example, oxalyl chloride, thionyl chloride, phosphorus pentachloride, phosphorus trichloride, etc.) at −20° C. to under reflux in the presence or absence of an organic solvent (for example, halogenated hydrocarbons such as chloroform and dichloromethane; ethers such as diethyl ether, 1,4-dioxane, and tetrahydrofuran; or acid amides such as N,N-dimethylformamide are used. These solvents may be used alone, or if required, in combination of two or more solvents in an appropriate mixing ratio of, for example, 1:1 to 1:10), and the resulting acid halide is then reacted with an amine at 0 to 40° C. in the presence of a base [for example, alkylamines such as triethylamine, tributylamine and diisopropylethylamine; aromatic amines such as N,N-dimethylaniline, pyridine, and 4-(dimethylamino)pyridine]. Alternatively, the acid halide can be reacted with an amine in an organic solvent (for example, diethyl ether, 1,4-dioxane, tetrahydrofuran, or the like may be used. These solvent may be used alone, or if required, in combination of two or more solvents thereof in an appropriate mixing ratio of, for example, 1:1 to 1:10) using an alkali aqueous solution (for example, aqueous sodium bicarbonate solution, aqueous sodium hydroxide solution, etc.) at 0 to 40° C.

(2) The method using a mixed anhydride is performed by reacting a carboxylic acid with an acid halide (for example, pivaloyl chloride, p-toluenesulfonyl chloride, methansulfonyl chloride, etc.) or an acid derivative (for example, ethyl chloroformate, isobutyl chloroformate, etc.) at 0 to 40° C. in the presence or absence of an organic solvent (for example, halogenated hydrocarbons such as chloroform and dichloromethane; ethers such as diethyl ether, tetrahydrofuran and 1,4-dioxane; acid amides such as N,N-dimethylformamide. These solvents may be used alone or in combination of two or more solvents thereof in an appropriate mixing ratio of, for example, 1:1 to 1:10) and in the presence of a base (e.g. pyridine, triethylamine, N,N-dimethylaniline, N,N-dimethylaminopyridine, diisopropylethylamine), and reacting the resulting mixed anhydride with an amine at 0° C. to 40° C. in an organic solvent (e.g. halogenated hydrocarbons such as chloroform and dichloromethane; ethers such as diethyl ether, tetrahydrofuran and 1,4-dioxane; acid amides such as N,N-dimethylformamide. These solvents may be used alone, or if required, in combination of two or more solvents thereof in an appropriate mixing ratio of, for example, 1:1 to 1:10).

(3) The method using a condensing agent is carried out by reacting a carboxylic acid with an amine at 0° C. to 40° C. in the presence or absence of an organic solvent (for example, halogenated hydrocarbons such as chloroform and dichloromethane; ethers such as diethyl ether, tetrahydrofuran and 1,4-dioxane; acid amides such as N,N-dimethylformamide are used. These solvents may be used alone, or if required, in combination of two or more solvents thereof in an appropriate mixing ratio of, for example, 1:1 to 1:10) with or without a base (e.g. alkylamines such as triethylamine, tributylamine and diisopropylethylamine; aromatic amines such as N,N-dimethylaniline, pyridine, 4-(dimethylamino)pyridine), using a condensing agent [e.g. 1,3-dicyclohexylcarbodiimide (DCC), 1-ethyl-3-[3-(dimethylamino)propyl]carbodiimide (EDC), 1,1′-carbonyldiimidazole (CDI), 2-chloro-1-methylpyridinium iodide, 1-propanephosphoric acid cyclic anhydride (PPA)], and using or not using 1-hydroxybenzotriazole (HOBt).

These reactions (1), (2) and (3) are all performed preferably under anhydrous conditions in an inert gas atmosphere (for example, argon, nitrogen).

3) The sulfonamidation reaction is known per se, and is performed by reacting a sulfonic acid with an acid halide (e.g. oxalyl chloride, thionyl chloride, phosphorus pentachloride, phosphorus trichloride, etc.) at −20° C. to under ref lux in the presence or absence of an organic solvent (for example, halogenated hydrocarbons such as chloroform and dichloromethane; ethers such as diethyl ether, tetrahydrofuran and 1,4-dioxane are used. These solvents may be used alone, or if required, in combination of two or more solvents thereof in an appropriate mixing ratio of, for example, 1:1 to 1:10), and reacting the resulting sulfonyl halide with an amine in an organic solvent (for example, halogenated hydrocarbons such as chloroform and dichloromethane; or ethers such as diethyl ether, tetrahydrofuran and 1,4-dioxane are used. These solvents may be used alone, or if required, in combination of two or more solvents thereof in an appropriate mixing ratio of, for example, 1:1 to 1:10) in the presence of a base (e.g. alkylamines such as triethylamine, tributylamine, and diisopropylethylamine; aromatic amines such as N,N-dimethylaniline, pyridine and 4-(dimethylamino)pyridines) at 0° C. to 40° C.

(B) Among the compounds represented by formula (I), a compound represented by formula (I-B): 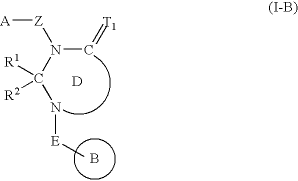
wherein R¹ and R² each independently represents a hydrogen atom or a substituent of ring D (the same as defined above), and other symbols have the same meanings as defined above, can be prepared by subjecting a compound represented by formula (IV): 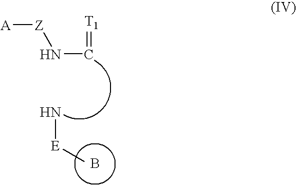
wherein all symbols have the same meanings as defined above, and a compound represented by formula (V): 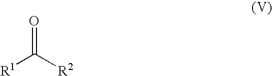
wherein all symbols have the same meanings as defined above, to a cyclization reaction.

This cyclization reaction is known per se, and is performed by reacting an amine with a carbonyl compound (e.g. formaldehyde, acetaldehyde, acetone, methyl ethyl ketone, etc.) at 0° C. to 100° C. in a solvent (for example, water; alcohols such as methanol, ethanol, n-propanol, and isopropanol; aromatic hydrocarbons such as benzene, toluene, and xylene; halogenated hydrocarbons such as dichloromethane, and chloroform; saturated hydrocarbons such as hexane, heptane, and cyclohexane; ethers such as diethyl ether, tetrahydrofuran, and 1,4-dioxane; nitriles such as acetonitrile; sulfoxides such as dimethyl sulfoxide; acid amides such as N,N-dimethylformamide; or esters such as ethyl acetate are employed. These solvents may be used alone, or if necessary, in combination with two or more solvents thereof in an appropriate mixing ratio of, for example, 1:1 to 1:10.

(C) Among the compounds represented by formula (I) according to the present invention, a compound when Z is a bond, —C(=T)- is —C(═O)— or an optionally substituted methylene group, and K is a nitrogen atom, i.e. a compound represented by formula (I-c): 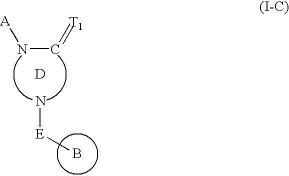
wherein all symbols have the same meanings as defined above, can be prepared by subjecting a compound represented by formula (VI-c): 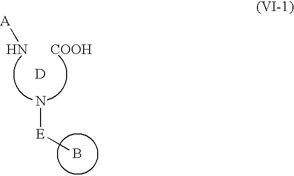
wherein all symbols have the same meanings as defined above, to a similar amidation reaction as described above, or by subjecting a compound represented by formula (VI-2): 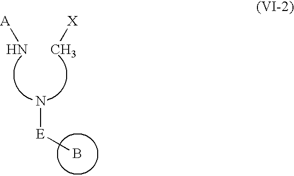
wherein all symbols have the same meanings as defined above, to a similar alkylation reaction as described above, or by reacting a compound represented by formula (VI-3): 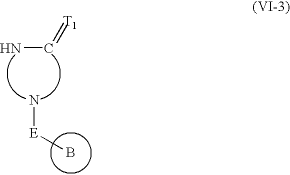
wherein all symbols have the same meanings as defined above, with a compound represented by formula (III-2):
X-A (III-2)
wherein all symbols have the same meanings as defined above.

The reaction of a compound represented by formula (VI-3) with a compound represented by formula (III-2) is carried out by alkylation reaction in the case where the alkylene group represented by A is bonded to X, or amidation reaction in the case where the carbonyl group represented by A is bonded to X, or sulfonamidation reaction in the case where the sulfonyl group represented by A is bonded to X. These reactions can be performed in a similar method to the method as described above.

(D) The compound represented by formula (I) according to the present invention can be prepared by subjecting a compound represented by formula (VII): 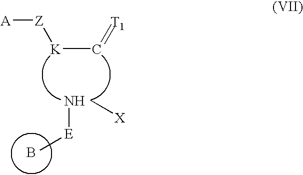
wherein all symbols have the same meanings as defined above, or a compound represented by formula (VIII): 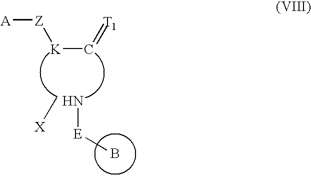
wherein all symbols have the same meanings as defined above, or a compound represented by formula (IX): 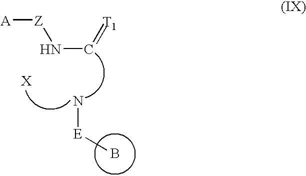
wherein all symbols have the same meanings as defined above, to an alkylation reaction.

The alkylation reaction can be carried out in a similar manner to the method as described above.

(E) Among the compounds represented by formula (I), a compound when T represents a sulfur atom or other substituent contains a thioxo group can be prepared by subjecting a compound obtained by the above method (A) to (D) to a thiocarbonylation reaction.

The thiocarbonylation reaction is performed by the known method or according to the method similar to the known method. For example, the reaction is carried out at 0° C. to 150° C. in an organic solvent (e.g. toluene, diethyl ether, dichloromethane, chloroform, 1,4-dioxane, tetrahydrofuran, etc.) in the presence of a thionylating reagent (e.g. Lawesson's reagent (2,4-bis(4-methoxyphenyl)-1,3-dithia-2,4-diphosphethane-2,4-disulfide), diphosphorus pentasulfide, etc.).

(F) Though it is easily understood by those skilled in the art, in the case the compounds represented by formula (I) according to the present invention and the compounds represented by formulae (I-B), (I-C), (II), (III-1), (III-2), (IV), (V), (VI-1), (VI-2), (VI-3), (VII), (VIII) and (IX) used as starting materials or reagents contain a hydroxy group, a carboxyl group, an amino group or a mercapto group, such compounds can be prepared by subjecting to the reaction of (A) to (E) after appropriate protection of the said group in advance, and then removing the protecting group for hydroxy, carboxyl, amino or mercapto group.

As the amino-protecting group, there are exemplified benzyloxycarbonyl group, t-butoxycarbonyl group, allyloxycarbonyl (Alloc) group, 1-methyl-1-(4-biphenyl)ethoxycarbonyl (Bpoc) group, trifluoroacetyl group, 9-fluorenylmethoxycarbonyl group, benzyl (Bn) group, p-methoxybenzyl group, benzyloxymethyl (BOM) group, 2-(trimethylsilyl)ethoxymethyl (SEM) group, and the like. As the hydroxy-protecting group, there are exemplified methyl group, trityl group, methoxymethyl (MOM) group, 1-ethoxyethyl (EE) group, methoxyethoxymethyl (MEM) group, 2-tetrahydropyranyl(THP) group, trimethylsilyl (TMS) group, triethylsilyl (TES) group, t-butyldimethylsilyl (TBDMS) group, t-butyldiphenylsilyl (TBDPS), acetyl (Ac) group, pivaloyl group, benzoyl group, benzyl (Bn) group, p-methoxybenzyl group, allyloxycarbonyl (Alloc) group, and 2,2,2-trichloroethoxycarbonyl (Troc) group, and the like.

As the mercapto-protecting group, there are exemplified benzyl group, methoxybenzyl group, methoxymethyl (MOM) group, 2-tetrahydropyranyl (THP) group, diphenylmethyl group, acetyl (Ac) group, and the like.

As the carboxyl-protecting group, there are exemplified methyl group, ethyl group, t-butyl group, allyl group, phenacyl group, benzyl group, and the like.

In addition to the above protecting groups for carboxyl, hydroxy, amino, or mercapto groups, there is no particular limitation so long as it can be easily and selectively removed. For example, protecting groups described in Protective Groups in Organic Synthesis (T. W. Greene, John Wiley & Sons Inc., 1999) also can be used.

The deprotection method for the protecting groups of carboxyl, hydroxy, amino, and mercapto groups is well known. Examples of such deprotetion are
(1) alkali hydrolysis
(2) deprotection under acidic conditions
(3) deprotection by hydrogenolysis
(4) deprotection using a metal complex
(5) deprotection using a metal, and
(6) deprotection of silyl groups.

Details of these deprotection methods are hereinafter illustrated.

(1) Deprotection by alkali hydrolysis such as deprotection of trifluoroacetyl group is performed in an organic solvent (e.g. methanol, tetrahydrofuran, 1,4-dioxane, etc.) at 0° C. to 40° C., using an alkali hydroxide (e.g. sodium hydroxide, potassim hydroxide, lithium hydroxide, etc.), an alkaline earth metal hydroxide (e.g. barium hydroxide, calcium hydroxide, etc.) or a carbonate (e.g. sodium carbonate, potassium carbonate, etc.) or an aqueous solution thereof or a mixture thereof.

(2) The deprotection under acidic conditions such as deprotection of t-butoxycarbonyl, trityl, and the like is carried out at 0° C. to 100° C. with an organic acid (e.g. acetic acid, trifluoroacetic acid, methanesulfonic acid, etc.) or an inorganic acid (e.g. hydrochloric acid, sulfuric acid, etc.) or a mixture thereof (e.g. hydrogen bromide/acetic acid) in water or an organic solvent (e.g. dichloromethane, chloroform, 1,4-dioxane, ethyl acetate, anisole, etc.).

(3) The deprotection by hydrogenolysis such as deprotection of benzyl, benzhydryl, benzyloxycarbonyl, allyloxycarbonyl, and the like is carried out at 0° C. to 200° C. in a solvent [ethers (e.g. tetrahydrofuran, 1,4-dioxane, dimethoxyethane, diethyl ether, etc.), alcohols (e.g. methanol, ethanol, etc.), benzenes (e.g. benzene, toluene, etc.), ketones (e.g. acetone, methyl ethyl ketone, etc.), nitriles (e.g. acetonitrile, etc.), amides (e.g. N,N-dimethylformamide, etc.), water, ethyl acetate, acetic acid or a mixture of two or more solvents thereof in the presence of a catalyst (e.g. palladium-carbon, palladium black, palladium hydroxide, platinum oxide, Raney-Ni, etc.) under a normal pressure or an increased pressure in a hydrogen stream or in the presence of ammonium formate.

(4) The deprotection using a metal, such as deprotection of allyloxycarbonyl group or the like, is performed at 0° C. to 40° C. in an organic solvent (e.g. dichloromethane, N,N-dimethylformamide, tetrahydrofuran, ethyl acetate, acetonitrile, 1,4-dioxane, ethanol, etc.), water or a mixture thereof in the presence of a trapping reagent (e.g. tributyltin hydride, triethylsilane, dimedone, morpholine, diethylamine, pyrrolidine, etc.), an organic acid (e.g. acetic acid, formic acid, 2-ethylhexanoic acid, etc.) and/or an organic acid salt (e.g. sodium 2-ethylhexanoate, potassium 2-ethylhexanoate, etc.) and in the presence or absence of a phosphine reagent (e.g. triphenylphosphine, etc.), using a metal complex [e.g. tetrakistriphenylphosphine palladium(0), bis(triphenylphosphine)palladium(II) dichloride, palladium(II) acetate, tris(triphenylphosphine)rhodium(I)].

(5) The deprotection using a metal is performed in an acidic solvent (e.g. acetic acid, a buffer of pH 4.2 to 7.2, or a mixture of a solvent thereof and an organic solvent such as tetrahydrofuran) in the presence of a zinc dust at 0 to 40° C. while applying ultrasonic waves, if required.

(6) The deprotection of the silyl group is carried out in a water-miscible organic solvent (e.g. tetrahydrofuran, acetonitrile, etc.) using tetrabutylammonium fluoride at 0° C. to 40° C.

Those skilled in the art can easily understand that the desired compounds of the present invention can be easily produced by selectively employing these deprotection methods. If necessary, conversion into desired non-toxic salts may be followed according to the known method.

The compounds represented by formula (I) of the present invention can be produced by the method described in Examples of the present invention, or the known method, for example, the method described in Comprehensive Organic Transformations: A Guide to Functional Group Preparations, 2^{nd} Edition (Richard C. Larock, John Wiley & Sons Inc, 1999), or a combination method thereof.

The compounds represented by formula (I-B), (I-C), (II), (III-1), (III-2), (IV), (V), (VI-1), (VI-2), (VI-3), (VII), (VIII) and (IX) as the starting material or the reagent to be used are known per se, or can be easily produced by the method described in, for example, Comprehensive Organic Transformations: A Guide to Functional Group Preparations, 2nd Edition (Richard C. Larock, John Wiley & Sons Inc, 1999), or a combination method thereof.

In each reaction of the present invention, a reagent appropriately carried on a solid carrier of polymers (e.g. polystyrene, polyacrylamide, polypropylene, polyethylene glycol, etc.) may be used.

The end products of the present invention can be purified by the conventional purification means such as distillation under normal pressure or reduced pressure, high performance liquid chromatography with silica gel or magnesium silicate, thin layer chromatography, or column chromatography, or wasing or recrystallization. Such purification may be carried out in each reaction or may be performed after several reactions.

The heating reaction in each reaction of the present invention may be performed using a water bath, an oil bath, a sand bath or a microwave, though it is apparent to those skilled in the art.

### PHARMACOLOGICAL ACTIVITY OF THE COMPOUNDS OF THE PRESENT INVENTION

Except for the pharmacological test described in Examples, there are exemplified the following methods. p38 MAP kinase inhibitory activity of the compounds of the present invention can be proven by these methods.

(a) Study on p38 αMAP Kinase Inhibitory Activity

Using activation transcription factor 2 (activating transcription factor 2; ATF-2, Cell Signaling Inc., #9224L) which is a substrate of p38 αMAP kinase, the inhibitory effect of the compound of the present invention on the ATF-2 phosphorylation by recombinant human p38 αMAP kinase (Upstate Biotechnology Inc., #14-251) was studied by the Western-blotting method using the anti-phosphorylated ATF-2 antibody (Cell Signaling Inc., #9221L). In other words, 10 μL of a solution of the compound of the present invention at a known concentration is added to 10 μL of the kinase buffer (Cell Signaling Inc., #9802) containing recombinant human p38 αMAP kinase (100 ng/tube) and pre-incubated for 10 minutes at 30° C. Then, 20 μL of adenosine triphosphate (ATP)/ATF-2 mixture is added, and after the incubation of 30 minutes at 30° C., 20 μL of SDS buffer (187.5 mM Tris/6% SDS/30% glycerol/150 mM DTT/0.03% bromophenol blue) is added to stop the enzyme reaction. After heating at 100° C. for 5 minutes, mixing and centrifugation are performed. After remixing, 20 μL of the sample is subjected to an electrophoresis on SDS-PAGE gel (10 to 20%, Daiichi Pure Chemicals Co., Ltd.). After the electrophoresis, blotting is performed on PVDF membrane (Sequi-Blot (proprietary name), 0.2 μm, BIO-RAD) by a conventional method. After that, the PVDF membrane is treated with Block Ace (Snow Brand Milk Products Co., Ltd.) (at room temperature, for 1 hour). After reacted with the anti-phosphorylated ATF-2 antibody for 1.5 hours, the membrane is washed with TBS-T solution (0.02M Tris/0.137M NaCl/0.05% Tween 20, pH 7.6). Furthermore, the reaction with a secondary antibody (anti-rabbit IgG, horseradish peroxide linked whole antibody, Amersham LIFE SCIENCE) is carried out for 1 hour. After washing with TBS-T solution, phosphorylated ATF-2 is detected using Western blotting detection reagent (Amersham Pharmacia Biotech).

(b) Mouse Cytokine-Producing Model

By the method shown below, the in vivo effect of the compounds of the present invention can be proven. The medium used for administering the compound of the present invention can be any medium so long as it is safe and is able to suspend or dissolve into an orally administerable form. For example, such medium includes methylcellulose, carboxymethylcellulose, hydroxypropylcellulose, hydroxypropylmethycellulose, propylene glycol, polyethylene glycol, sugar, sugar alcohol, edible oil, distilled water, physiological saline, and a mixture thereof, all of which have been used for administering a compound to an animal by those skilled in the art.

[Experimental Method]

The compound of the present invention suspended in 0.5% methylcellulose (MC) is orally administered to a male Balb/c mouse (Charles River Japan, Inc.), and after 0.5 hour, lipopolysaccharide (LPS, 055:B5, Difco) is intraperitoneally administered at the dose of 1 mg/kg (5 animals/group). MC (0.5%) is orally administered to a control group (5 animals). Ninety minutes after the LPS treatment, heparinized blood collection is performed via the abdominal main vein under anesthesia with ether, and blood plasma is obtained by centrifugation (12,000 rpm, 3 minutes, 4° C.). The obtained blood plasma sample is stored at −80° C. until it is used. TNF-α and IL-6 in the blood plasma are measured using ELISA kits from R&D Inc. (#MTA00) and Endogen Inc. (#EM2IL6), respectively.

[Toxicity]

Toxicity of the compound represented by formula (I) of the present invention is sufficiently low, and it was confirmed to be safe enough for use as pharmaceuticals.

[Application for Pharmaceuticals]

Since the compounds represented by formula (I) of the present invention suppresse p38 MAP kinase activation in animals including human, particularly in human, they are expected to be useful in the prevention and/or the treatment of cytokine-mediated diseases such as various inflammatory diseases (e.g. inflammation, dermatitis, atopic dermatitis, hepatitis, nephritis, glomerulonephritis, pancreatitis, psoriasis, gout, Addison's disease, arthrititis (e.g. articular rheumatism, osteoarthritis, rhumatoid spondylitis, gouty arthritis, synovitis, etc.), inflammatory ocular diseases, inflammatory pulmonary diseases (e.g. chronic pneumonia, silicosis, pulmonary sarcoidosis, pulmonary tuberculosis, adult respiratory distress syndrome (ARDS), severe acute respiratory syndrome (SARS)), inflammatory bowel diseases (e.g. Crohn's disease, ulcerative colitis, etc.), allergic diseases (e.g. allergic dermatitis, allergic rhinitis, etc.), autoimmune disease, autoimmune hemolytic anemia, systemic lupus eryhtematosus, rheumatism, Castleman's disease, immune rejection accompanying transplantation, (e.g. graft versus host reaction, etc.), etc.], central nervous system disorders [e.g. central neuropathy (e.g. cerebrovascular disease such as cerebral hemorrhage and cerebral infarction, head trauma, spinal cord injury, cerebral edema, multiple sclerosis, etc.), neuronal degeneration (Alzheimer's disease, Parkinson's disease, amyotrophic lateral sclerosis (ALS), AIDS dementia, etc.), meningitis, Creutzfeldt-Jakob syndrome, etc.], respiratory diseases [e.g. athma, chronic obstructive pulmonary disease (COPD), etc.], cardiovascular diseases [e.g. angina pectoris, heart failure, congestive heart failure, acute heart failure, chronic heart failure, myocardial infarction, acute myocardial infarction, myocardial infarction prognosis, atrial myxoma, arteriosclerosis, hypertension, dialysis-induced hypotension, thrombosis, disseminated intravascular coagulation (DIC), reperfusion injury, restenosis after percutaneous transluminal coronary angioplasty (PTCA), etc.], urinary diseases [e.g. renal failure, etc.], metabolic diseases and endocrine diseases [e.g. diabetes, etc.], bone diseases [e.g. osteoporosis, etc.], cancerous diseases [e.g. malignant tumor (e.g. tumor growth and metastasis, multiple myeloma, plasma cell leukemia, carcinemia, etc.], infectious diseases [e.g. viral infection, (e.g. cytomegalovirus infection, influenza virus infection, herpes virus infection, corona virus infection, etc.), cachexia associated with infections, cachexia caused by acquired immune deficiency syndrome (AIDS), toxemia (e.g. sepsis, septic shock, endotoxin shock, gram negative bacterial sepsis, toxic shock syndrome, severe acute respiratory syndrome (SARS) accompanying virus infection, etc.)].

Among subtypes (α, β, β₂, γ, δ) of p38 MAP kinase, the compounds of the present invention include compounds selectively inhibiting subtype α, and compounds inhibiting other subtypes other than subtype α.

The compounds represented by formula (I) of the present invention, or their pharmacologically acceptable salts (e.g. acid addition salts), their N-oxides, their solvates (e.g. hydrate), or prodrugs thereof can be usually administered systemically or topically in the form of oral or parenteral administration.

Since the compounds represented by formula (I) of the present invention are safe and have low toxicity, they can be administered to a human or a mammal other than humans (e.g. rat, mouse, rabbit, sheep, pig, cow, cat, dog, monkey, etc.).

Although the dose varies depending on age, body weight, symptom, therapeutic effect, administration route and treatment time, the dose for a human adult is generally within a range of 1 mg to 1000 mg per administration that is orally administered up to several times a day, or within a range of 1 mg to 100 mg per administration that is parenterally or preferebaly intravenously administered up to several times a day or intravenously administered over a period of continuous 1 to 24 hours a day.

As mentioned above, the dose to be prescribed depends upon various conditions, and thus there are cases in which doses lower than the range as specified above may be enough or doses greater than the range as specified above may be required.

In the administration of the compounds of the present invention, they are used as solid preparations or liquid preparations for oral administration, or as injections, external preparations or suppositories for parenteral administration.

In the production of these compositions, the compounds of the present invention, their salts, N-oxides, or solvates, or prodrugs thereof are not limited to a substantially chemically pure single substance, they may contain impurities (for example, by-products derived from the production process, solvents, starting materials, or decomposition products) so long as such impurities are within an acceptable range as a pharmaceutical bulk.

The solid preparations for oral administration include tablets, pills, capsules, dispersible powders, granules, and the like. The capsules include hard capsules and soft capsules.

In such solid preparations for oral use, one or more of the active compound(s) may be admixed solely or with diluents (e.g. lactose, mannitol, glucose, microcrystalline cellulose, starch, etc.), binders (e.g. hydroxypropylcellulose, polyvinylpyrrolidone, magnesium aluminometasilicate, etc.), disintegrators (e.g. cellulose calcium glycolate, etc.), lubricants (e.g. magnesium stearate, etc.), stabilizers, solubilizers (e.g. glutamic acid, aspartic acid, etc.), and then formulated into a preparation in the conventional manner. When necessary, such preparations may be coated with a coating agent such as sucrose, gelatin, hydroxypropylcellulose, hydroxypropylmethyl cellulose phthalate, etc.) or they may be coated with two or more coating layers. Furthermore, the solid preparations for oral use include capsules of absorbable materials like gelatin.

The liquid preparations for oral administration include pharmaceutically acceptable aqueous solutions, suspensions, emulsions, syrups, elixirs, and the like. In such preparations, one or more of the active compound(s) may be dissolved, suspended or emulsified in a commonly used diluent (e.g. purified water, ethanol or a mixture thereof, etc.). Besides such diluents, said compositions may also contain wetting agents, suspending agents, emulsifiers, sweetening agents, flavouring agents, perfumes, preservatives, and buffers, and the like.

Injections for parenteral administration include any injection and also include instillation solutions. For example, such injections for parenteral administration include intramuscular injection, subcutaneous injection, intracutaneous injection, intraarterial injection, intraveneous injection, intraperitoneal injection, intraspinally injection, and intraveneous instillation.

Injections for parenteral administration include solutions, suspensions, emulsions, and solid injectable which are dissolved or suspended in a solvent immediately before use. The injections are used by dissolving, suspending or emulsifying one or more of the active compound(s) in a diluent. Said diluents may contain distilled water for injection, physiological saline, vegetable oil, alcohol (e.g. propylene glycol, polyethylene glycol, ethanol, etc.), and a combination thereof. Further, the injections may contain stabilizers, solubilizers (e.g. glutamic acid, aspartic acid, polysorbate 80 (registered trade mark), etc.), suspending agents, emulsifiers, soothing agents, buffers, preservatives, etc. The injections are sterilized in the final formulation step or prepared by sterile procedure. The injections may also be formulated into sterile solid preparation, for example, by freeze-drying, and may be used after sterilized or dissolved in sterile injectable water or other sterile diluent(s) immediately before use.

Other preparations for parenteral administration may contain one or more active compounds, and as such compositions, there are exemplified conventionally formulated external solutions, ointments, pastes, inhalations, sprays, suppositories, or vaginal pessaries.

Sprays may contain stabilizers such as sodium hydrogen sulfite, and buffers capable of imparting isotonicity, including isotonic agents such as sodium chloride, sodium citrate and citric acid. The formulation method for sprays is described in detail in US patents 2868691 and 3095355.

The compounds represented by formula (I) or the non-toxic salts thereof may be administered in combination with other drugs for the purpose of: 1) complement and/or enhancement of preventing and/or treating effect of the compound, 2) improvement of dynamics and absorption of the compound, and lowering of dose, and/or 3) alleviation of side effect of the compound.

Also, a combination of the compounds of the present invention may be administered as a combination drug for the purpose of: 1) complement and/or enhancement of preventing and/or treating effect of the combination other drugs, 2) improvement of dynamics and absorption of the combination other drugs, and lowering of dose, and/or 3) alleviation of side effect of the combination other drugs.

The compounds represented by formula (I) may be administered in combination with other drugs as a preparation in one drug product comprising these components, or may be administered separately. When they are administered independently, they may be administered simultaneously or with time lag. Administeration with time lag includes the method of administering first the compounds represented by formula (I) and subsequently administering other drugs, and the method of administering first the other drug and subsequently administering the compound represented by formula (I), and they may be administered in the same route or not.

There is no limitation on the diseases on which the above combination drugs have a preventing and/or treatment effect, so long as the preventing and/or treatment effect of the compound of the formula (I) is complemented and/or enhanced in the disease.

The weight proportion of the compounds represented by formula (I) and the other drugs is not specifically limited.

Arbitrary two or more of the other drugs may be administered in combination.

Examples of the other drugs for compensating for and/or enhancing the preventive and/or treatment effect of the compounds represented by formula (I) include not only those which have so far been found but also those which will be found on the basis of the aforementioned mechanism.

Other agents to complement and/or enhance a prevention and/or a treatment effect of the compound represented by formula (I) on rheumatoid arthritis, osteoarthritis, arthritis or the like include a steroidal agent, an elastase inhibitor, a cannabinoid-2 receptor stimulating agent, a prostaglandin, a prostaglandin synthase inhibitor, a phosphodiesterase inhibitor, a metalloproteinase inhibitor, an adhesion molecule inhibitor, an anti-cytokine protein preparation (e.g. an anti-TNF-α preparation, an anti-IL-1 preparation, an anti-IL-6 preparation, etc.), a cytokine inhibitor, an immunomodulator, a disease modifying anti-rheumatic agent, a non-steroidal anti-inflammatory agent, c-Jun N-terminal kinase inhibitor, and the like.

Other agents to complement and/or enhance prevention and/or treatment effect of the compound represented by formula (I) on inflammatory bowel disease, Crohn's disease or ulcerative colitis include a steroidal agent, an elastase inhibitor, a cannabinoid-2 receptor stimulating agent, a prostaglandin, a prostaglandin synthase inhibitor, a phosphodiesterase inhibitor, a metalloproteinase inhibitor, an adhesion molecule inhibitor, an anti-cytokine protein preparation, a cytokine inhibitor, an immunomodulator, a leukotoriene receptor antagonist, an anticholinergic agent, a 5-lipoxygenase inhibitor, a nitric monooxide synthase inhibitor, an interleukin 8 antagonist, a poly(ADP)-ribose polymerase inhibitor, a mitochondrial benzodiazepine receptor agonist, an anti-oxidation agent, a local anesthetic, an agent for digestive tract ulcer, a defense factor enhancing agent, mesalazine, salazosulfapyridine and the like.

Other agents to complement and/or enhance prevention and/or treatment effect of the compound represented by formula (I) on asthma, chronic pulmonary inflammatory diseases or adult respiratory distress syndrome (ARDS) include asteroidal agent, an elastase inhibitor, a cannabinoid-2 receptor stimulating agent, a prostaglandin, a prostaglandin synthase inhibitor, a phosphodiesterase inhibitor, a metalloproteinase inhibitor, an adhesion molecule inhibitor, a leukotoriene receptor antagonist, an anticholinergic agent, a thromboxane A₂ receptor antagonist, a thromboxane synthase inhibitor, a β₂ adrenergic receptor stimulating agent, a xanthine derivative, an expectorant agent, an antibiotic, an anti-histamine agent, an anti-cytokine protein preparation, a cytokine inhibitor, a forskolin preparation, a mediator release inhibitor, and the like.

Examples of the steroidal agent include clobetasol propionate, diflorasone diacetate, fluocinonide, mometasone furoate, betamethasone dipropionate, betamethasone butyrate propionate, betamethasone valerate, difluprednate, diflucortolone valerate, amcinonide, halcinonide, dexamethasone, dexamethasone propionate, dexamethasone valerate, dexamethasone acetate, hydrocortisone acetate, hydrocortisone butyrate, hydrocortisone butyrate propionate, deprodone propionate, prednisolone valerate acetate, fluocinolone acetonide, beclomethasone dipropionate, triamcinolone acetonide, flumethasone pivalate, alclometasone propionate, clobetasone butyrate, prednisolone, fludroxycortide, cortisone acetate, hydrocortisone, hydrocortisone sodium phosphate, hydrocortisone sodium succinate, fludrocortisone acetate, prednisolone acetate, prednisolone sodium succinate, prednisolone butylacetate, prednisolone sodium phosphate, halopredone acetate, methylprednisolone, methylprednisolone acetate, methylprednisolone sodium succinate, triamcinolone, triamcinolone acetate, dexamethasone sodium phosphate, dexamethasone palmitate, paramethasone acetate, betamethasone, fluticasone propionate, budesonide, flunisolide, ST-126P, ciclesonide, dexamethasone palmithionate, mometasone furanecarbonate, prasterone sulfonate, deflazacort, methylprednisolone suleptanate, and methylprednisolone sodium succinate, and the like.

Examples of the elastase inhibitor include ONO-5046, ONO-6818, MR-889, PBI-1101, EPI-HNE-4, R-665, ZD-0892, ZD-8321, GW-311616, DMP-777, L-659286, L-658758, L-680833, L-683845, AE-3763, and the like.

Examples of the prostaglandins (hereinafter referred to as PG) are a PG receptor agonist and a PG receptor antagonist, and the like.

Examples of the PG receptors include a PGE receptor (EP₁, EP₂, EP₃, EP₄), a PGD receptor (DP, GRTH2), a PGF receptor (FP), PGI receptor (IP), and a TX receptor (TP), and the like.

Examples of the prostaglandin synthase inhibitors include salazosulfapyridine, mesalazine, osalazine, 4-aminosalicylic acid, JTE-522, auranofin, carprofen, difenpiramide, flunoxaprofen, flubiprofen, indometacin, ketoprofen, lornoxicam, loxoprofen, meloxicam, oxaprozin, parsalmide, piproxen, piroxicam, piroxicambetadex, piroxicam cinnamate, tropine indometacinate, zaltoprofen, and pranoprofen, and the like.

Examples of the phosphodiesterase inhibitors include a PDE4 inhibitor such as rolipram, cilomilast (proprietary name:Ariflo), Bay19-8004, NIK-616, roflumilast (BY-217), cipamfylline (BRL-61063), atizoram (CP-80633), SCH-351591, YM-976, V-11294A, PD-168787, D-4396, and IC-485, and a PDE5 inhibitor such as sildenafil, and the like.

Examples of the adhesion molecule inhibitors include α₄ integrin, and the like.

Examples of the anti-TNF-α preparations include a preparation containing an anti-TNF-α antibody, a soluble TNF-α receptor, an anti-TNF-α receptor antibody or a protein bound to a soluble TNF-α, such as a preparation containing infliximab or etanercept, or the like.

Examples of the anti-IL-1 preparations include a preparation containing an anti-IL-1 antibody, a soluble IL-1 receptor, IL-1Ra or an anti-IL-1 receptor antibody, such as a preparation containing anakinra or the like.

Examples of the anti-IL-6 preparations include a preparation containing an anti-IL-6 antibody, a soluble IL-6 receptor, or an anti-IL-6 receptor antibody, such as a preparation containing MRA or the like.

Examples of the immunomodulators include methotrexate, cyclosporin, ascomycin, leflunomide, bucillamine, salazosulfapyridine, azathioprine, tacrolimus, and cyclophosphamide, and the like.

Examples of the disease modifying anti-rheumatic agents include gold thioglucose, sodium aurothiomalate, auranofin, chloroquine, actarit, D-penicillamine preparation, lobenzarit disodium, bucillamine, hydroxychloroquine, and salazosulfapyridine, and the like.

Examples of the non-steroidal anti-inflammatory agents include sasapyrine, sodium salicylate, aspirin, aspirin dialuminate combinations, diflunisal, indometacin, suprofen, ufenamate, dimethylisopropylazulene, bufexamac, felbinac, diclofenac, tolmetin sodium, clinoril, fenbufen, napumetone, proglumetacin, indometacin farnesil, acemetacin, proglumetacin maleate, amfenac sodium, mofezolac, etodolac, ibuprofen, ibuprofen piconol, naproxen, flurbiprofen, flurbiprofen axetil, ketoprofen, fenoprofen calcium, tiaprofen, oxaprozin, pranoprofen, loxoprofen sodium, alminoprofen, zaltoprofen, mefenamic acid, mefenamic acid aluminium, tolfenamic acid, floctafenine, ketophenylbutazone, oxyphenbutazone, piroxicam, tenoxicam, ampiroxicam, Napageln ointment, epirizole, tiaramide hydrochloride, tinoridine hydrochloride, emorfazone, sulpyrine, migrenin, Saridon, Sedes G, Amipylo-N, sorbone, a pyrine drug for flu, acetaminophen, phenacetin, dimetotiazine mesilate, simetride combinations, a non-pyrine drug for flu.

Examples of the leukotoriene receptor antagonists include pranlukast hydrate, montelukast, zafirlukast, seratrodast, MCC-847, KCA-757, CS-615, YM-158, L-740515, CP-195494, LM-1484, RS-635, A-93178, S-36496, BIIL-284, and ONO-4057, and the like.

Examples of the anti-cholinergic agents include ipratropium bromide, oxitropium bromide, flutropium bromide, cimetropium bromide, temiverine, thiotropium bromide, and revatropate (UK-112166), and the like.

Examples of the local anesthetics include cocaine hydrochloride, procaine hydrochloride, lidocaine, dibucaine hydrochloride, and tetracaine hydrochloride, and the like.

Examples of the defence factor enhancing agents include sucralfate, aldioxa, teprenone, cetraxate hydrochloride, and ornoprostil, and thhe like.

Examples of the thromboxane A₂ receptor antagonists include seratrodast, ramatroban, domitroban calcium hydrate, and KT-2-962, and the like.

Examples of the thromboxane synthase inhibitors include ozagrel hydrochloride, and imitrodast sodium, and the like.

Examples of the β₂ adrenergic receptor stimulating agents include fenoterol hydrobromide, salbutamol sulfate, terbutaline sulfate, formoterol fumarate, salmeterol xinafoate, isoproterenol sulfate, orciprenaline sulfate, chlorprenaline sulfate, epinephrine, trimetoquinol hydrochloride, hexoprenalinemesyl sulfate, procaterol hydrochloride, tulobuterol hydrochloride, tulobuterol, pirbuterol hydrochloride, clenbuterol hydrochloride, mabuterol hydrochloride, ritodrine hydrochloride, bambuterol, dopexamine hydrochloride, meluadrine tartrate, AR-C68397, levosalbutamol, R,R-formoterol, KUR-1246, KUL-7211, AR-C89855, and S-1319, and the like.

Examples of the xanthine derivatives include aminophylline, theophylline, doxophylline, cipamfylline, and diprophylline, and the like.

Examples of the expectorant agents include foeniculated ammonia spirit, sodium hydrogen carbonate, bromhexine hydrochloride, carbocysteine, ambroxol hydrochloride, ambroxol hydrochloride sustained preparation, methylcysteine hydrochloride, acetylcysteine, L-ethylcysteine hydrochloride, and tyloxapol, and the like.

Examples of the antibiotics include cefuroxime sodium, meropenem trihydrate, netilmicin sulfate, sisomicin sulfate, ceftibuten, PA-1806, IB-367, tobramycin, PA-1420, doxorubicin, astromicin sulfate, and cefetamet pivoxil hydrochloride, and the like. Examples of the antibiotics as an inhalation include PA-1806, IB-367, tobramycin, PA-1420, doxorubicin, astromicin sulfate, cefetamet pivoxil hydrochloride.

Examples of the anti-histamine agents include ketotifen fumarate, mequitazine, azelastine hydrochloride, oxatomide, terfenadine, emedastine difumarate, epinastine hydrochloride, astemizole, ebastine, cetirizine hydrochloride, bepotastine, fexofenadine, loratadine, desloratadine, olopatadine hydrochloride, TAK-427, ZCR-2060, NIP-530, mometasone furoate, mizolastine, BP-294, and last, auranofin, and acrivastine, and the like.

Examples of the cytokine inhibitors include any one of non-protein preparations which can block the action of cytokines, containing a MAP kinase inhibitor, a gene regulating agent, a cytokine production inhibitor, a TNF-α converting enzyme inhibitor, an IL-1β converting enzyme inhibitor, an IL-6 antagonist, an IL-8 antagonist, a chemokine antagonist, a gene therapy agent, and an anti-sense compound, and the like. The MAP kinase inhibitor includes, for example, PD-98059 and the like. The gene regulating agent includes an inhibitor to molecules involved in signal transmission, such as NF-κB, IKK-1, IKK-2, and AP-1, and the like. The cytokine production inhibitor includes, for example, suplatast tosilate (proprietary name: IPD), T-614, SR-31747, and sonatimod, and the like. The chemokine antgonist includes, for example, ONO-4128 and the like. The gene therapy agent includes, for example, a gene therapy agent for accelerating expression of genes having antiinflammatory action, such as interleukin 4, interleukin 10, a soluble IL-1 receptor and a soluble TNF-α receptor, and the like.

Examples of the mediator release inhibitors include, for example tranilast, sodium cromoglicate, amlexanox, repirinast, ibudilast, dazanolast, and pemirolast potassium, and the like.

Examples of the c-Jun N-terminal kinase inhibitors include compounds described in WO 00/35906, WO 00/35909, WO 00/35921, WO 00/64872, and WO 00/75118, and the like.

### EFFECT OF THE PRESENT INVENTION

Since the compounds represented by formula (I) of the present invention has p38 MAP kinase inhibitory activity and their toxicity is low, said compounds are very useful in the prevention and/or treatment of cytokine-mediated diseases such as inflammatory diseases, central nervous system diseases, respiratory diseases, cardiovascular disease, urinary disease, metabolic diseses, endocrine diseases, bone diseases, cancerous diseases, infections, and the like.

### BEST MODE FOR CARRYING OUT THE INVENTION

The following Examples and Reference Examples are provided to illustrate the present invention in detail, but are not to be construed as limiting the invention.

The solvents in the parenthesis described in chromatography separation and TLC show an elution solvent or a developing solvent used, and the ratio is given in volume.

Amorphous substance was confirmed using a polarization microscope.

### REFERENCE EXAMPLE 1

### N-(4-bromo-2,6-dichlorophenyl)acrylamide

A solution of (4-bromo-2,6-dichlorophenyl)amine (4 g) and acryloyl chloride (1.4 mL) in dichloromethane (10 mL) was stirred overnight at room temperature. To the solution were added water and ethyl acetate at 0 degree. The organic layer was washed with a saturated aqueous solution of sodium bicarbonate and a saturated aqueous solution of sodium chloride, subsequently, dried over an anhydrous magnesium sulfate and then concentrated. The residue was washed with diisopropylether to give the title compound (1.96 g) having the following physical data.

TLC: Rf 0.25 (hexane:ethyl acetate=5:1);

NMR(CDCl₃): δ 7.55 (s, 2H), 7.11 (br, 1H), 6.51-6.44 (m, 1H), 6.40-6.20 (m, 1H), 5.85 (d, J=10.5 Hz, 1H).

### REFERENCE EXAMPLE 2

### N-(4-bromo-2,6-dichlorophenyl)-3-[(2,4-difluorobenzyl)amino]propanamide

A solution of the compound prepared in Reference Example 1 (290 mg) and (2,4-difluorobenzyl)amine (420 mg) in toluene (1 mL) was stirred for 16 hours at 80 degrees. To the solution were added water and ethyl acetate. The organic layer was washed with a saturated aqueous solution of sodium chloride, dried over an anhydrous magnesium sulfate and then concentrated. The residue was purified by column chromatography on silica gel (ethyl acetate) to give the title compound having the following physical data.

TLC: Rf 0.20 (hexane:ethyl acetate=1:1);

NMR(CDCl₃): δ 10.35 (br, 1H), 7.52 (s, 2H), 7.30-7.20 (m, 1H), 6.90-6.70 (m, 2H), 3.90 (s, 2H), 3.05 (t, J=8.7 Hz, 2H), 2.57 (t, J=8.7 Hz, 2H).

### Example 1

### 3-(4-bromo-2,6-dichlorophenyl)-1-(2,4-difluorobenzyl)tetrahydropyrimidin-4(1H)-one

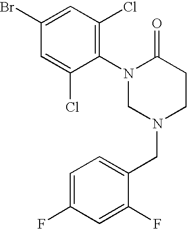

To a solution of the compound prepared in Reference Example 2 in tetrahydrofuran (3 mL) was added 37% aqueous solution of formaldehyde (2 mL). The mixture was stirred for 30 minutes at room temperature. To the mixture were added water and ethyl acetate. The organic layer was washed with a saturated aqueous solution of sodium chloride, dried over an anhydrous magnesium sulfate and concentrated to give the compound of the present invention (230 mg) having the following physical data.

TLC: Rf 0.54 (hexane:ethyl acetate=1:1);

NMR(CDCl₃): δ 7.56 (s, 2H), 7.43 (m, 1H), 6.85 (m, 2H), 4.34 (s, 2H), 4.05 (s, 2H), 3.22 (t, J=6.77 Hz, 2H), 2.71 (t, J=6.41 Hz, 2H).

### EXAMPLE 1(1)-1(18)

Using corresponding amine instead of (4-bromo-2,6-dichlorophenyl)amine, using corresponding amine instead of (2,4-difluorobenzyl)amine, subjecting to a conversion reaction to hydrochloride, if necessary, the following compounds were obtained by the same procedures as a series of reactions of Reference Example 1→Reference Example 2→Example 1.

### EXAMPLE 1(1)

### 1-(4-aminobenzyl)-3-(2,6-dimethylphenyl)tetrahydropyrimidin-4(1H)-one dihydrochloride

TLC: Rf 0.60 (ethyl acetate:methanol:ammonia water=16:3:1);

NMR(DMSO-d₆): δ 7.50-7.42 (m, 2H), 7.20-7.00 (m, 5H), 4.98 (brs, 2H), 4.30 (brs, 2H), 3.50-3.40 (m, 2H), 2.90-2.80 (m, 2H), 2.16 (s, 6H).

### EXAMPLE 1(2)

### 1-(4-aminobenzyl)-3-(2-methylphenyl)tetrahydropyrimidin-4(1H)-one dihydrochloride

TLC: Rf 0.72 (ethyl acetate: methanol:ammonia water=16:3:1);

NMR(DMSO-d₆): δ 7.50-7.42 (m, 2H), 7.38-7.22 (m, 4H), 7.15-7.10 (m, 2H), 4.98 (m, 2H), 4.30 (m, 2H), 3.50-3.40 (m, 2H), 2.90-2.80 (m, 2H), 2.17 (s, 3H).

### EXAMPLE 1(3)

### N-[3-t-butyl-1-(4-methylphenyl)-1H-pyrazol-5-yl]-N′-(4-{[3-(1-ethylpropyl)-4-oxotetrahydro-1(2H)-pyrimidinyl]methyl}phenyl)urea

TLC: Rf 0.53 (ethyl acetate);

NMR(CDCl₃): δ 0.82 (t, J=7.41 Hz, 6H), 1.33 (s, 9H), 1.34 (m, 4H), 2.36 (m, 5H), 2.80 (t, J=6.50 Hz, 2H), 3.62 (s, 2H), 3.77 (s, 2H), 4.31 (m, 1H), 6.41 (s, 1H), 7.26 (m, 9H), 7.83 (s, 1H).

### EXAMPLE 1(4)

### N-[3-t-butyl-1-(4-methylphenyl)-1H-pyrazol-5-yl]-N′-(4-{[4-oxo-3-(1-piperidinyl)tetrahydro-1(2H)-pyrimidinyl]methyl}phenyl)urea

TLC: Rf 0.65 (chloroform:methanol=9:1);

NMR(CDCl₃): δ 1.32 (m, 11H), 1.50 (m, 4H), 2.33 (m, 5H), 2.77 (t, J=6.41 Hz, 2H), 3.05 (m, 4H), 3.63 (s, 2H), 3.99 (s, 2H), 6.41 (s, 1H), 7.09 (s, 1H), 7.27 (m, 8H), 7.71 (s, 1H).

### EXAMPLE 1(5)

### N-[3-t-butyl-1-(4-methylphenyl)-1H-pyrazol-5-yl]-N′-{4-[(4-oxo-3-phenyltetrahydro-1(2H)-pyrimidinyl)methyl]phenyl}urea

TLC: Rf 0.28 (ethyl acetate);

NMR(CDCl₃): δ 1.32 (s, 9H), 2.33 (s, 3H), 2.41 (t, J=6.50 Hz, 2H), 3.06 (t, J=6.50 Hz, 2H), 3.76 (s, 2H), 4.29 (s, 2H), 6.36 (s, 1H), 7.16 (m, 10H), 7.30 (m, 4H), 7.70 (s, 1H).

### EXAMPLE 1(6)

### N-(3-t-butyl-1-methyl-1H-pyrazol-5-yl)-N′-(4-{[3-(2-methylphenyl)-4-oxotetrahydro-1(2H)-pyrimidinyl]methyl}phenyl)urea

TLC: Rf 0.28 (dichloromethane:methanol=9:1);

NMR(CDCl₃): δ 1.28 (s, 9H), 2.20 (s, 3H), 2.67 (m, 2H), 3.12 (t, J=6.59 Hz, 2H), 3.52 (s, 3H), 3.82 (m, 2H), 4.21 (m, 2H), 6.06 (s, 1H), 7.04 (m, 1H), 7.18 (m, 8H), 7.58 (s, 1H).

### EXAMPLE 1(7)

### N-[3-t-butyl-1-(4-methylphenyl)-1H-pyrazol-5-yl]-N′-(4-{[3-(2-methoxyphenyl)-4-oxotetrahydro-1(2H)-pyrimidinyl]methyl}phenyl)urea

TLC: Rf 0.55 (chloroform:methanol=9:1);

NMR(CDCl₃): δ 1.33 (s, 9H), 2.35 (s, 3H), 2.40 (t, J=6.41 Hz, 2H), 3.10 (t, J=6.41 Hz, 2H), 3.70 (s, 3H), 3.81 (s, 2H), 4.22 (s, 2H), 6.36 (s, 1H), 6.84 (m, 2H), 7.00 (dd, J=7.87, 1.65 Hz, 1H), 7.10 (s, 1H), 7.20 (m, 7H), 7.31 (m, 2H), 7.66 (s, 1H).

### EXAMPLE 1(8)

### N-[3-t-butyl-1-(4-methylphenyl)-1H-pyrazol-5-yl]-N′-(4-{[3-(2-ethylphenyl)-4-oxotetrahydro-1(2H)-pyrimidinyl]methyl}phenyl)urea

TLC: Rf 0.58 (chloroform:methanol=9:1);

NMR(CDCl₃): δ 1.16 (t, J=7.60 Hz, 3H), 1.33 (s, 9H), 2.35 (s, 3H), 2.46 (m, 4H), 3.03 (m, 2H), 3.77 (m, 2H), 4.14 (m, 2H), 6.36 (s, 1H), 6.97 (d, J=7.51 Hz, 1H), 7.03 (s, 1H), 7.19 (m, 9H), 7.31 (m, 2H), 7.55 (s, 1H).

### EXAMPLE 1(9)

### N-[3-t-butyl-1-(4-methylphenyl)-1H-pyrazol-5-yl]-N′-{4-[(4-oxo-3-propyltetrahydro-1(2H)-pyrimidinyl)methyl]phenyl}urea

TLC: Rf 0.51 (chloroform:methanol=9:1);

NMR(CDCl₃): δ 0.84 (t, J=7.32 Hz, 3H), 1.33 (s, 9H), 1.47 (m, 2H), 2.28 (t, J=6.41 Hz, 2H), 2.34 (s, 3H), 2.85 (t, J=6.41 Hz, 2H), 3.16 (m, 2H), 3.63 (s, 2H), 3.91 (s, 2H), 6.41 (s, 1H), 7.19 (m, 5H), 7.31 (m, 4H), 7.86 (s, 1H).

### EXAMPLE 1(10)

### N-[3-t-butyl-1-(4-methylphenyl)-1H-pyrazol-5-yl]-N′-(4-{[3-(2-ethylbutyl)-4-oxotetrahydro-1(2H)-pyrimidinyl]methyl}phenyl)urea

TLC: Rf 0.51 (chloroform:methanol=9:1);

NMR(CDCl₃): δ 0.81 (t, J=7.41 Hz, 6H), 1.23 (m, 4H), 1.32 (s, 9H), 1.46 (m, 1H), 2.27 (t, J=6.41 Hz, 2H), 2.33 (s, 3H), 2.83 (t, J=6.41 Hz, 2H), 3.13 (d, J=7.51 Hz, 2H), 3.62 (s, 2H), 3.89 (s, 2H), 6.41 (s, 1H), 7.18 (m, 4H), 7.30 (m, 5H), 7.98 (s, 1H).

### EXAMPLE 1(11)

### N-[3-t-butyl-1-(4-methylphenyl)-1H-pyrazol-5-yl]-N′-(4-{[4-oxo-3-(2-propylphenyl)tetrahydro-1(2H)-pyrimidinyl]methyl}phenyl)urea

TLC: Rf 0.48 (chloroform:methanol=9:1);

NMR(CDCl₃): δ 0.93 (t, J=7.32 Hz, 3H), 1.33 (s, 9H), 1.54 (m, 2H), 2.34 (s, 3H), 2.42 (m, 4H), 3.02 (m, 2H), 3.77 (m, 2H), 4.14 (m, 2H), 6.35 (s, 1H), 6.97 (d, J=7.32 Hz, 2H), 7.18 (m, 9H), 7.31 (m, 2H), 7.53 (s, 1H).

### EXAMPLE 1(12)

### N-[3-t-butyl-1-(4-methylphenyl)-1H-pyrazol-5-yl]-N′-(4-{[3-(2,3-dimethylphenyl)-4-oxotetrahydro-1(2H)-pyrimidinyl]methyl}phenyl)urea

TLC: Rf 0.41 (chloroform:methanol=9:1);

NMR(CDCl₃): δ 1.32 (s, 9H), 2.02 (s, 3H), 2.16 (s, 3H), 2.34 (s, 3H), 2.39 (m, 2H), 3.03 (m, 2H), 3.76 (m, 2H), 4.11 (m, 2H), 6.36 (s, 1H), 6.79 (m, 1H), 7.02 (m, 2H), 7.16 (m, 7H), 7.29 (m, 2H), 7.66 (s, 1H).

### EXAMPLE 1(13)

### N-[3-t-butyl-1-(4-methylphenyl)-1H-pyrazol-5-yl]-N′-(4-{[3-(2,4-dimethylphenyl)-4-oxotetrahydro-1(2H)-pyrimidinyl]methyl}phenyl)urea

TLC: Rf 0.41 (chloroform:methanol=9:1);

NMR(CDCl₃): δ 1.33 (s, 9H), 2.09 (s, 3H), 2.23 (s, 3H), 2.34 (s, 3H), 2.40 (m, 2H), 3.02 (m, 2H), 3.76 (m, 2H), 4.06 (d, J=10.80 Hz, 1H), 4.17 (m, 1H), 6.37 (s, 1H), 6.82 (d, J=7.87 Hz, 1H), 6.94 (m, 2H), 7.07 (s, 1H), 7.17 (m, 6H), 7.29 (m, 2H), 7.65 (s, 1H).

### EXAMPLE 1(14)

### N-[3-t-butyl-1-(4-methylphenyl)-1H-pyrazol-5-yl]-N′-(4-{[3-[2-(methylthio)phenyl]-4-oxotetrahydro-1(2H)-pyrimidinyl]methyl}phenyl)urea

TLC: Rf 0.43 (ethyl acetate);

NMR(CDCl₃): δ 1.32 (s, 9H), 2.30 (m, 7H), 2.47 (m, 1H), 3.08 (t, J=6.59 Hz, 2H), 3.81 (d, J=13.18 Hz, 1H), 3.90 (m, 1H), 4.22 (m, 2H), 6.35 (s, 1H), 7.11 (m, 13H), 7.66 (s, 1H).

### EXAMPLE 1(15)

### N-[3-t-butyl-1-(4-methylphenyl)-1H-pyrazol-5-yl]-N′-(4-{[3-(1-methyl-1H-pyrazol-5-yl)-4-oxotetrahydro-1(2H)-pyrimidinyl]methyl}phenyl)urea

TLC: Rf 0.41 (chloroform:methanol=9:1);

NMR(CDCl₃): δ 1.33 (s, 9H), 2.32 (s, 3H), 2.60 (t, J=6.41 Hz, 2H), 3.14 (t, J=6.41 Hz, 2H), 3.66 (s, 3H), 3.81 (s, 2H), 4.23 (s, 2H), 6.00 (d, J=2.01 Hz, 1H), 6.37 (s, 1H), 6.76 (s, 1H), 7.13 (m, J=8.24 Hz, 2H), 7.25 (m, 6H), 7.39 (d, J=2.01 Hz, 1H), 7.45 (s, 1H).

### EXAMPLE 1(16)

### N-[3-t-butyl-1-(4-methylphenyl)-1H-pyrazol-5-yl]-N′-(4-{[3-{4-[4-(dimethylamino)-1-butyn-1-yl]-2-methylphenyl}-4-oxotetrahydro-1(2H)-pyrimidinyl]methyl}phenyl)urea

TLC: Rf 0.26 (dichloromethane:methanol=10:1);

NMR(CDCl₃): δ 1.33 (s, 9H), 2.13 (s, 3H), 2.27 (s, 6H), 2.36 (s, 3H), 2.56 (m, 6H), 3.08 (s, 2H), 3.79 (d, J=3.48 Hz, 2H), 4.15 (m, 2H), 6.38 (s, 1H), 6.93 (d, J=7.87 Hz, 2H), 7.22 (m, 8H), 7.33 (d, J=8.24 Hz, 2H), 7.40 (s, 1H).

### EXAMPLE 1(17)

### methyl 2-[3-{4-[({[3-t-butyl-1-(4-methylphenyl)-1H-pyrazol-5-yl]amino}carbonyl)amino]benzyl}-6-oxotetrahydro-1(2H)-pyrimidinyl]benzoate

TLC: Rf 0.57 (chloroform:methanol=9:1);

NMR(CDCl₃): δ 1.30 (s, 9H), 2.32 (m, 5H), 3.12 (t, J=5.68 Hz, 2H), 3.75 (s, 3H), 3.83 (m, 2H), 4.30 (m, 2H), 6.33 (s, 1H), 7.00 (dd, J=7.78, 1.01 Hz, 1H), 7.22 (m, 10H), 7.43 (m, 1H), 7.89 (dd, J=7.78, 1.56 Hz, 1H), 7.95 (s, 1H).

### EXAMPLE 1(18)

### N-(3-t-butyl-1-phenyl-1H-pyrazol-5-yl)-N′-(4-{[3-(2-methylphenyl)-4-oxotetrahydro-1(2H)-pyrimidinyl]methyl}phenyl)urea

TLC: Rf 0.55 (dichloromethane:methanol=9:1);

NMR(CDCl₃): δ 1.35 (s, 9H), 2.16 (s, 3H), 2.47 (m, 2H), 3.06 (t, J=6.13 Hz, 2H), 3.76 (d, J=13.18 Hz, 1H), 3.82 (d, J=13.18 Hz, 1H), 4.09 (d, J=10.80 Hz, 1H), 4.22 (d, J=10.80 Hz, 1H), 6.37 (s, 1H), 6.87 (m, 1H), 6.98 (m, 1H), 7.30 (m, 13H).

### REFERENCE EXAMPLE 3

### 3-({[(2,6-dimethylphenyl)amino]carbonyl}amino)propanoic acid

To a solution of 2-isocyanate-1,3-dimethylbenzene (1.5 g) in N,N-dimethylformamide (10 mL) was added β-alanine (900 mg) and the mixture was stirred overnight at room temperature. An appeared solid was collected by suction, washed with ethyl acetate and water to give the title compound having the following physical data.

TLC: Rf 0.45 (ethyl acetate);

NMR(DMSO-d₆): δ 12.20 (s, 1H), 7.53 (s, 1H), 7.03-7.00 (m, 3H), 6.05 (brs, 1H), 3.24 (q, J=6.6 Hz, 2H), 2.38 (t, J=6.6 Hz, 2H), 2.12 (s, 6H).

### REFERENCE EXAMPLE 4

### 3-(2,6-dimethylphenyl)dihydropyrimidine-2,4(1H,3H)-dione

To a solution of the compound prepared in Reference Example 3 (1 g) in N,N-dimethylformamide (10 mL) were added 1-(3-dimethylaminopropyl)-3-ethylcarbodiimide (1.6 g), 1-hydroxybenzotriazole (1.1 g) and triethylamine (1.2 mL). The mixture was stirred overnight at room temperature. To the mixture were added water and ethyl acetate. The organic layer was washed with a saturated aqueous solution of sodium chloride, dried over an anhydrous magnesium sulfate and then concentrated. The residue was washed with diethylether to give the title compound (270 mg) having the following physical data.

TLC: Rf 0.38 (ethyl acetate);

NMR(CDCl₃): δ 7.23-7.18 (m, 1H), 7.15-7.12 (m, 2H), 6.02 (brs, 1H), 3.55 (dt, J=3.0, 6.6 Hz, 2H), 2.90 (t, J=6.6 Hz, 2H), 2.15 (s, 6H).

### EXAMPLE 2

### 3-(2,6-dimethylphenyl)-1-(4-nitrobenzyl)dihydropyrimidine-2,4(1H,3H)-dione

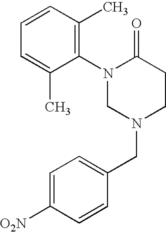

To a solution of the compound prepared in Reference Example 4 (200 mg) in N,N-dimethylformamide (3 mL) was added sodium hydride (40 mg) at 0 degree. The mixture was stirred for 10 minutes at room temperature. To the reaction mixture was added 1-(bromomethyl)-4-nitrobenzene (200 mg) and the mixture was stirred for 15 minutes at room temperature. To the reaction mixture was added hydrochloric acid, and then an appeared solid was collected by suction. The obtained solid was dissolved in ethyl acetate, dried over an anhydrous magnesium sulfate and concentrated to give the compound of the present invention (300 mg) having the following physical data.

TLC: Rf 0.64 (ethyl acetate);

NMR(CDCl₃): δ 8.26-8.22 (m, 2H), 7.54-7.50 (m, 2H), 7.25-7.14 (m, 3H), 4.78 (s, 2H), 3.51 (t, J=6.9 Hz, 2H), 2.96-2.88 (m, 2H), 2.12 (s, 6H).

### REFERENCE EXAMPLE 5

### benzyl {3-[(4-bromo-2,6-dichlorophenyl)amino]-3-oxopropyl}carbamate

To N-[(benzyloxy)carbonyl]-β-alanine (2 g) was added thionyl chloride (2 mL). The mixture was stirred for 30 minutes at room temperature and then concentrated. To the residue was added (4-bromo-2,6-dichlorophenyl)amine (2 g) and the mixture was stirred for 5 minutes at room temperature. To the reaction mixture was added dichloromethane (10 mL) and the mixture was stirred for 1 hour at room temperature. To the mixture were added water and ethyl acetate. The organic layer was washed with a saturated aqueous solution of sodium bicarbonate and a saturated aqueous solution of sodium chloride, subsequently, dried over an anhydrous magnesium sulfate and then concentrated to give the title compound (2.1 g) having the following physical data.

TLC: Rf 0.35 (hexane:ethyl acetate=1:1);

NMR(CDCl₃): δ 7.54 (s, 2H), 7.38-7.30 (m, 5H), 7.15 (br, 1H), 5.35 (br, 1H), 5.10 (s, 2H), 3.62-3.55 (m, 2H), 2.75-2.65 (m, 2H).

### REFERENCE EXAMPLE 6

### 3-amino-N-(2,6-dichlorophenyl)propanamide hydrobromide

The compound prepared in Reference Example 5 (700 mg) was dissolved in methanol (10 mL) and tetrahydrofuran (10 mL). To the solution was added 10% palladium on carbon (25 mg). The mixture was stirred for 2 hours at room temperature under an atmosphere of hydrogen. The reaction mixture was filtered. The filtrate was concentrated to give the title compound (492 mg) having the following physical data.

TLC: Rf 0.50 (ethyl acetate:methanol:ammonia water=16:3:1);

NMR(DMSO-d₆): δ 10.10 (br, 1H), 7.75 (br, 2H), 7.56-7.53 (m, 2H), 7.38-7.33 (m, 1H), 3.07 (t, J=7.2 Hz, 2H), 2.75 (t, J=7.2 Hz, 2H).

### REFERENCE EXAMPLE 7

### 3-(2,6-dichlorophenyl)tetrahydropyrimidin-4(1H)-one

Using the compound prepared in Reference Example 6 instead of the compound prepared in Reference Example 2, the title compound having the following data was obtained by the same procedure of Example 1.

TLC: Rf 0.33 (ethyl acetate:triethylamine=19:1);

NMR(CDCl₃): δ 7.42-7.39 (m, 2H), 7.26-7.21 (m, 1H), 4.43 (s, 2H), 3.31 (t, J=6.3 Hz, 2H), 2.61 (t, J=6.3 Hz, 2H), 2.00 (brs, 1H).

### EXAMPLE 2(1)-2(23)

Using the compound prepared in Reference Example 4 or following compounds of (1)-(7) [(1) the compound which is obtained by the same procedures as a series of reactions of Reference Example 3→Reference Example 4, using corresponding isocyanate instead of 2-isocyanate-1,3-dimethylbenzene, (2) the compound which is obtained by the same procedures as a series of reactions of Reference Example 5→Reference Example 6→Reference Example 7, using corresponding amine instead of (4-bromo-2,6-dichlorophenyl)amine, (3) 3-(2-methylphenyl)-2,4-imidazolidinedione, (4) 1-(2-methylphenyl)-2-piperazinone, (5) 1-phenyl-1,4-diazepan-2-one, (6) 4-(2-methylphenyl)-1,4-diazepan-5-one and (7) 1-(2-methylbenzoyl)piperazine] instead of it, using corresponding halide (alkyl halide and sulfonyl halide) instead of 1-(bromomethyl)-4-nitrobenzene, the compounds were obtained by the same procedure of Example 2.

### EXAMPLE 2(1)

### 3-(2,6-dichlorophenyl)-1-(2,4-difluorobenzyl)dihydropyrimidine-2,4(1H,3H)-dione

TLC: Rf 0.33 (ethyl acetate:hexane=3:7);

NMR(CDCl₃): δ 7.50-7.39 (m, 3H), 7.29 (m, 1H), 6.94-6.80 (m, 2H), 4.71 (s, 2H), 3.56 (t, J=6.3 Hz, 2H), 2.91 (t, J=6.3 Hz, 2H).

### EXAMPLE 2(2)

### 3-(2,6-dichlorophenyl)-1-(2-fluorobenzyl)tetrahydropyrimidin-4(1H)-one

TLC: Rf 0.44 (hexane:ethyl acetate=1:1);

NMR(CDCl₃): δ 7.48 (m, 1H), 7.39 (m, J=8.42 Hz, 2H), 7.22 (m, 4H), 4.40 (s, 2H), 4.12 (s, 2H), 3.24 (m, 2H), 2.73 (m, 2H).

### EXAMPLE 2(3)

### 2-{[3-(2,6-dichlorophenyl)-4-oxotetrahydropyrimidin-1(2H)-yl]methyl}benzyl(3-t-butyl-1-methyl-1H-pyrazol-5-yl)carbamate

TLC: Rf 0.45 (ethyl acetate);

NMR(CDCl₃): δ 7.32 (m, 7H), 6.44 (m, 1H), 6.03 (s, 1H), 5.40 (s, 2H), 4.35 (s, 2H), 4.11 (s, 2H), 3.65 (s, 3H), 3.16 (t, J=6.41 Hz, 2H), 2.67 (t, J=6.41 Hz, 2H), 1.26 (s, 9H).

### EXAMPLE 2(4)

### N-[3-t-butyl-1-(4-methylphenyl)-1H-pyrazol-5-yl]-2-(4-{[3-(2-methylphenyl)-4-oxotetrahydro-1(2H)-pyrimidinyl]methyl}phenyl)acetamide

TLC: Rf 0.38 (ethyl acetate);

NMR(CDCl₃): δ 1.32 (s, 9H), 2.23 (s, 3H), 2.37 (s, 3H), 2.66 (m, 2H), 3.13 (t, J=6.50 Hz, 2H), 3.65 (s, 2H), 3.88 (s, 2H), 4.23 (m, 2H), 6.59 (s, 1H), 7.19 (m, 13H).

### EXAMPLE 2(5)

### 3-(2-methylphenyl)-1-(4-nitrobenzyl)dihydro-2,4(1H,3H)-pyrimidinedione

TLC: Rf 0.33 (ethyl acetate:hexane=2:1);

NMR(CDCl₃): δ 2.15 (s, 3H), 2.92 (m, 2H), 3.50 (m, 2H), 4.70 (d, J=14.4 Hz, 1H), 4.83 (d, J=14.4 Hz, 1H), 7.09 (m, 1H), 7.32 (m, 3H), 7.52 (d, J=8.7 Hz, 2H), 8.24 (d, J=8.7 Hz, 2H).

### EXAMPLE 2(6)

### t-butyl (2-{[3-(2-methylphenyl)-2,4-dioxotetrahydro-1(2H)-pyrimidinyl]methyl}benzyl)carbamate

TLC: Rf 0.26 (ethyl acetate:hexane=1:1);

NMR(CDCl₃): δ 1.43 (s, 9H), 2.17 (s, 3H), 2.84 (t, J=6.77 Hz, 2H), 3.43 (m, 2H), 4.34 (d, J=6.04 Hz, 2H), 4.69 (d, J=15.20 Hz, 1H), 4.84 (d, J=15.20 Hz, 1H), 5.04 (m, 1H), 7.12 (m, 1H), 7.31 (m, 7H).

### EXAMPLE 2(7)

### 1-[2-(aminomethyl)benzyl]-3-(2-methylphenyl)dihydro-2,4(1H,3H)-pyrimidinedione

NMR(CDCl₃): δ 1.98 (s, 3H), 2.96 (t, J=6.87 Hz, 2H), 3.80 (t, J=6.87 Hz, 2H), 4.13 (m, 2H), 4.64 (s, 2H), 6.96 (d, J=7.32 Hz, 1H), 7.18 (m, 3H), 7.37 (m, 4H), 7.83 (m, 3H).

### EXAMPLE 2(8)

### 4-(4-nitrobenzyl)-1-phenyl-1,4-diazepan-2-one

TLC: Rf 0.40 (ethyl acetate:hexane=2:1);

NMR(CDCl₃): δ 1.97 (m, 2H), 3.03 (m, 2H), 3.67 (s, 2H), 3.87 (m, 2H), 3.93 (s, 2H), 7.25 (m, 3H), 7.40 (m, 2H), 7.56 (d, J=8.7 Hz, 2H), 8.18 (d, J=8.7 Hz, 2H).

### EXAMPLE 2(9)

### 3-(2-methylphenyl)-1-(4-nitrobenzyl)-2,4-imidazolidinedione

TLC: Rf 0.33 (ethyl acetate:hexane=1:1);

NMR(CDCl₃): δ 2.24 (s, 3H), 4.00 (s, 2H), 4.73 (d, J=15.6 Hz, 1H), 4.79 (d, J=15.6 Hz, 1H), 7.18 (m, 1H), 7.35 (m, 3H), 7.51 (d, J=8.7 Hz, 2H), 8.28 (d, J=8.7 Hz, 2H).

### EXAMPLE 2(10)

### 3-(2-methoxyphenyl)-1-(4-nitrobenzyl)dihydro-2,4(1H,3H)-pyrimidinedione

TLC: Rf 0.24 (ethyl acetate:hexane=2:1);

NMR(CDCl₃): δ 2.90 (t, J=6.6 Hz, 2H), 3.48 (m, 2H), 3.83 (s, 3H), 4.65 (d, J=15.6 Hz, 1H), 4.89 (d, J=15.6 Hz, 1H), 7.02 (m, 1H), 7.04 (m, 1H), 7.17 (m, 1H), 7.39 (m, 1H), 7.52 (d, J=8.7 Hz, 2H), 8.23 (d, J=8.7 Hz, 2H).

### EXAMPLE 2(11)

### methyl 2-[4-(4-nitrobenzyl)-7-oxo-1,4-diazepan-1-yl]benzoate

TLC: Rf 0.46 (ethyl acetate);

NMR(CDCl₃): δ 2.24 (s, 3H), 2.85 (m, 6H), 3.64 (m, 1H), 3.74 (s, 2H), 3.86 (m, 1H), 7.07 (m, 1H), 7.24 (m, 3H), 7.55 (d, J=8.7 Hz, 2H), 8.21 (d, J=8.7 Hz, 2H).

### EXAMPLE 2(12)

### methyl 2-[3-(4-nitrobenzyl)-2,6-dioxotetrahydro-1(2H)-pyrimidinyl]benzoate

TLC: Rf 0.30 (toluene:ethyl acetate=1:1);

NMR(CDCl₃): δ 2.92 (m, 2H), 3.47 (m, 1H), 3.63 (m, 1H), 3.84 (s, 3H), 4.57 (d, J=15.6 Hz, 1H), 4.97 (d, J=15.6 Hz, 1H), 7.27 (m, 1H), 7.49 (m, 1H), 7.56 (d, J=8.7 Hz, 2H), 7.65 (m, 1H), 8.15 (m, 1H), 8.23 (d, J=8.7 Hz, 2H).

### EXAMPLE 2(13)

### 1-(2-methylphenyl)-4-(4-nitrobenzyl)-2-piperazinone

TLC: Rf 0.29 (toluene:ethyl acetate=2:1);

NMR(CDCl₃): δ 2.23 (s, 3H), 2.86 (m, 2H), 3.32 (d, J=15.74 Hz, 1H), 3.40 (d, J=15.74 Hz, 1H), 3.47 (m, 1H), 3.67 (m, 1H), 3.74 (s, 2H), 7.15 (m, 1H), 7.25 (m, 3H), 7.57 (d, J=8.79 Hz, 1H), 8.23 (d, J=8.79 Hz, 1H).

### EXAMPLE 2(14)

### 1-(2-methylphenyl)-4-(3-nitrobenzyl)-2-piperazinone

TLC: Rf 0.26 (hexane:ethyl acetate=1:2);

NMR(CDCl₃): δ 2.23 (s, 3H), 2.86 (m, 2H), 3.30 (d, J=16.48 Hz, 1H), 3.39 (d, J=16.48 Hz, 1H), 3.48 (m, 1H), 3.67 (m, 1H), 3.74 (s, 2H), 7.16 (m, 1H), 7.26 (m, 3H), 7.54 (t, J=7.87 Hz, 1H), 7.72 (d, J=7.51 Hz, 1H), 8.17 (m, 1H), 8.27 (t, J=1.92 Hz, 1H).

### EXAMPLE 2(15)

### 3-benzyl-1-(4-nitrobenzyl)dihydro-2,4(1H,3H)-pyrimidinedione

TLC: Rf 0.30 (toluene:ethyl acetate=1:1);

NMR(CDCl₃): δ 2.75 (t, J=6.6 Hz, 2H), 3.32 (t, J=6.6 Hz, 2H), 4.72 (s, 2H), 5.01 (s, 2H), 7.30 (m, 4H), 7.40 (m, 3H), 8.19 (d, J=8.7 Hz, 2H).

### EXAMPLE 2(16)

### sodium 4-{[3-(2-methylphenyl)-4-oxotetrahydro-1(2H)-pyrimidinyl]methyl}benzoate

TLC: Rf 0.44 (dichloromethane:methanol=9:1);

NMR(CD₃OD): δ 2.20 (s, 3H), 2.62 (m, 2H), 3.17 (t, J=6.50 Hz, 2H), 3.96 (m, 2H), 4.26 (m, 2H), 7.11 (m, 1H), 7.23 (m, 3H), 7.40 (d, J=8.60 Hz, 2H), 7.91 (m, 2H).

### EXAMPLE 2(17)

### N-[3-t-butyl-1-(4-methylphenyl)-1H-pyrazol-5-yl]-4-{[3-(2-methylphenyl)-4-oxotetrahydro-1(2H)-pyrimidinyl]methyl}benzamide

TLC: Rf 0.58 (ethyl acetate);

NMR(CDCl₃): δ 1.38 (s, 9H), 2.22 (s, 3H), 2.42 (s, 3H), 2.66 (m, 2H), 3.15 (t, J=6.59 Hz, 2H), 3.94 (m, 2H), 4.22 (m, 2H), 6.77 (s, 1H), 7.06 (m, 1H), 7.23 (m, 3H), 7.32 (m, 2H), 7.40 (m, 2H), 7.48 (d, J=8.24 Hz, 2H), 7.69 (d, J=8.24 Hz, 2H), 7.96 (s, 1H).

### EXAMPLE 2(18)

### N-(3-t-butyl-1-methyl-1H-pyrazol-5-yl)-4-{[3-(2-methylphenyl)-4-oxotetrahydro-1(2H)-pyrimidinyl]methyl}benzamide

TLC: Rf 0.47 (ethyl acetate);

NMR(CDCl₃): δ 1.29 (s, 9H), 2.19 (s, 3H), 2.62 (m, 2H), 3.13 (t, J=6.41 Hz, 2H), 3.67 (s, 3H), 3.95 (m, 2H), 4.23 (m, 2H), 6.13 (s, 1H), 7.02 (m, 1H), 7.21 (m, 3H), 7.49 (d, J=8.24 Hz, 2H), 7.85 (d, J=8.24 Hz, 2H), 8.13 (s, 1H).

### EXAMPLE 2(19)

### ethyl (2E)-3-(4-{[3-(2-methylphenyl)-4-oxotetrahydro-1(2H)-pyrimidinyl]methyl}phenyl)acrylate

TLC: Rf 0.87 (chloroform:methanol=3:1);

NMR(CDCl₃): δ 1.34 (t, J=7.14 Hz, 3H), 2.23 (s, 3H), 2.68 (m, 2H), 3.16 (m, 2H), 3.91 (m, 2H), 4.25 (m, 4H), 6.43 (d, J=16.11 Hz, 1H), 7.08 (m, 1H), 7.23 (m, 3H), 7.40 (d, J=8.06 Hz, 2H), 7.51 (d, J=8.06 Hz, 2H), 7.67 (d, J=16.11 Hz, 1H).

### EXAMPLE 2(20)

### (2E)-N-[3-t-butyl-1-(4-methylphenyl)-1H-pyrazol-5-yl]-3-(4-{[3-(2-methylphenyl)-4-oxotetrahydro-1(2H)-pyrimidinyl]methyl}phenyl)acrylamide hydrochloride

TLC: Rf 0.45 (ethyl acetate);

NMR(DMSO-d₆): δ 1.28 (s, 9H), 2.17 (s, 3H), 2.33 (s, 3H), 2.75 (m, 2H), 3.45 (m, 2H), 4.37 (m, 2H), 4.66 (m, 2H), 6.40 (s, 1H), 6.87 (d, J=15.74 Hz, 1H), 7.30 (m, 8H), 7.54 (d, J=15.74 Hz, 1H), 7.64 (m, 4H), 10.09 (s, 1H).

### EXAMPLE 2(21)

### (2E)-N-(3-t-butyl-1-methyl-1H-pyrazol-5-yl)-3-(4-{[3-(2-methylphenyl)-4-oxotetrahydro-1(2H)-pyrimidinyl]methyl}phenyl)acrylamide hydrochloride

TLC: Rf 0.69 (chloroform:methanol=6:1);

NMR(DMSO-d₆): δ 1.22 (s, 9H), 2.18 (s, 3H), 2.80 (m, 2H), 3.48 (m, 2H), 3.70 (s, 3H), 4.41 (m, 2H), 4.70 (m, 2H), 6.26 (s, 1H), 7.05 (d, J=15.93 Hz, 1H), 7.25 (m, 4H), 7.63 (m, 5H), 10.43 (s, 1H).

### EXAMPLE 2(22)

### 3-(2-methylphenyl)-1-[(4-nitrophenyl)sulfonyl]tetrahydro-4(1H)-pyrimidinone

TLC: Rf 0.57 (ethyl acetate);

NMR(CDCl₃): δ 2.16 (s, 3H), 2.77 (m, 2H), 3.75 (m, 2H), 4.91 (s, 2H), 7.03 (m, 1H), 7.26 (m, 3H), 8.02 (d, J=8.7 Hz, 2H), 8.39 (d, J=8.7 Hz, 2H).

### EXAMPLE 2(23)

### 1-(2-methylbenzoyl)-4-(4-nitrobenzyl)piperazine

TLC: Rf 0.09 (toluene:ethyl acetate=4:1).

Example 3

### 3-(2,6-dimethylphenyl)-1-(4-aminobenzyl)dihydropyrimidine-2,4(1H,3H)-dione

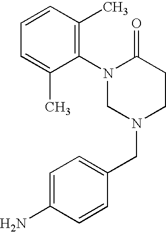

To a solution of the compound prepared in Example 2 (325 mg) in acetic acid (10 mL) were added water (1 mL) and iron powder(1 g). The mixture was stirred for 3 hours at room temperature. The reaction mixture was filtered. The filtrate was concentrated. To the obtained residue were added a saturated aqueous solution of sodium bicarbonate and ethyl acetate. The organic layer was washed with a saturated aqueous solution of sodium chloride, dried over an anhydrous magnesium sulfate and then concentrated to give the compound of the present invention (260 mg) having the following physical data.

TLC: Rf 0.19 (hexane:ethyl acetate=1:1);

NMR(CDCl₃): δ 7.22-7.11 (m, 5H), 6.68-6.50 (m, 2H), 4.56 (s, 2H), 3.41 (t, J=6.9 Hz, 2H), 2.82 (t, J=6.9 Hz, 2H), 2.12 (s, 6H).

### EXAMPLE 3(1)-3(11)

Using the compounds prepared in Example 2(5), 2(8), 2(9), 2(10), 2(11), 2(12), 2(13), 2(14), 2(15), 2(22) and 2(23) instead of the compound prepared in Example 2, the following compounds were obtained by the same procedure of Example 3.

### EXAMPLE 3(1)

### 1-(4-aminobenzyl)-3-(2-methylphenyl)dihydro-2,4(1H,3H)-pyrimidinedione

TLC: Rf 0.17 (ethyl acetate:hexane=2:1);

NMR(CDCl₃) δ 2.14 (s, 3H), 2.81 (m, 2H), 3.41 (m, 2H), 3.70 (brs, 2H), 4.45 (d, J=14.4 Hz, 1H), 4.63 (d, J=14.4 Hz, 1H), 6.66 (d, J=8.7 Hz, 2H), 7.11 (m, 3H), 7.29 (m, 3H).

### EXAMPLE 3(2)

### 4-(4-aminobenzyl)-1-phenyl-1,4-diazepan-2-one

TLC: Rf 0.51 (chloroform:methanol=9:1).

### EXAMPLE 3(3)

### 1-(4-aminobenzyl)-3-(2-methylphenyl)-2,4-imidazolidinedione

TLC: Rf 0.25 (ethyl acetate:hexane=1:1);

NMR(CDCl₃): δ 2.21 (s, 3H), 3.73 (brs, 2H), 3.89 (s, 2H), 4.50 (d, J=15.6 Hz, 1H), 4.55 (d, J=15.6 Hz, 1H), 6.69 (d, J=8.7 Hz, 2H), 7.11 (d, J=8.7 Hz, 2H), 7.16 (m, 1H), 7.32 (m, 3H).

### EXAMPLE 3(4)

### 1-(4-aminobenzyl)-3-(2-methoxyphenyl)dihydro-2,4(1H,3H)-pyrimidinedione

TLC: Rf 0.63 (chloroform:methanol=9:1).

### EXAMPLE 3(5)

### 1-(4-aminobenzyl)-4-(2-methylphenyl)-1,4-diazepan-5-one

TLC: Rf 0.23 (ethyl acetate:methanol=19:1);

NMR(CDCl₃): δ 2.22 (s, 3H), 2.77 (m, 6H), 3.52 (s, 2H), 3.59 (m, 1H), 3.81 (m, 1H), 6.66 (d, J=8.7 Hz, 2H), 7.07 (m, 1H), 7.11 (d, J=8.7 Hz, 2H), 7.21 (m, 3H).

### EXAMPLE 3(6)

### methyl 2-[3-(4-aminobenzyl)-2,6-dioxotetrahydro-1(2H)-pyrimidinyl]benzoate

TLC: Rf 0.19 (toluene:ethyl acetate=1:1).

### EXAMPLE 3(7)

### 4-(4-aminobenzyl)-1-(2-methylphenyl)-2-piperazinone

TLC: Rf 0.22 (hexane:ethyl acetate=1:8).

### EXAMPLE 3(8)

### 4-(3-aminobenzyl)-1-(2-methylphenyl)-2-piperazinone

TLC: Rf 0.12 (hexane:ethyl acetate=1:2).

### EXAMPLE 3(9)

### 1-(4-aminobenzyl)-3-benzyldihydro-2,4(1H,3H)-pyrimidinedione

TLC: Rf 0.44 (ethyl acetate:hexane=2:1).

### EXAMPLE 3(10)

### 1-[(4-aminophenyl)sulfonyl]-3-(2-methylphenyl)tetrahydro-4(1H)-pyrimidinone

TLC: Rf 0.36 (ethyl acetate);

NMR(CDCl₃): δ 2.17 (s, 3H), 2.68 (m, 2H), 3.65 (m, 2H), 4.20 (brs, 2H), 4.80 (s, 2H), 6.69 (d, J=8.7 Hz, 2H), 7.03 (m, 1H), 7.25 (m, 3H), 7.61 (d, J=8.7 Hz, 2H).

### EXAMPLE 3(11)

### 1-(2-methylbenzoyl)-4-(4-aminobenzyl)piperazine

TLC: Rf 0.48 (chloroform:methanol=9:1).

### EXAMPLE 4

### N-[3-t-butyl-1-(4-methylphenyl)-1H-pyrazol-5-yl]-N′-(4-{[3-(2,6-dimethylphenyl)-4-oxotetrahydropyrimidin-1(2H)-yl]methyl}phenyl)urea

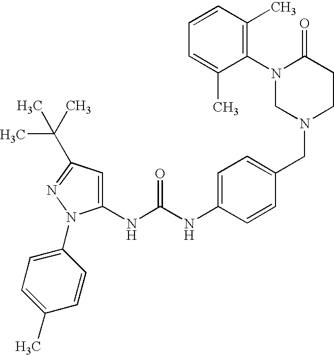

To a solution of the compound prepared in Example 1(1) (100 mg) in tetrahydrofuran (5 mL) was added triethylamine (70 μL) and the mixture was stirred for 30 minutes at room temperature. To the reaction mixture was added N-(3-t-butyl-1-methyl-1H-pyrazol-5-yl)-4-(dimethyliminio)pyridine-1(4H)-carboxyimidate (200 mg), and the mixture was stirred for 3 hours at room temperature. To the mixture were added water and ethyl acetate. The organic layer was washed with a saturated aqueous solution of sodium chloride, dried over an anhydrous magnesium sulfate and then concentrated. The residue was purified by column chromatography on silica gel (ethyl acetate) to give the compound of the present invention (40 mg) having the following physical data.

TLC: Rf 0.25 (ethyl acetate);

NMR(CDCl₃): δ 7.23 (m, 11H), 6.91 (m, 1H), 6.38 (s, 1H), 6.34 (s, 1H), 4.03 (s, 2H), 3.80 (s, 2H), 3.04 (t, J=6.32 Hz, 2H), 2.61 (t, J=6.41 Hz, 2H), 2.37 (s, 3H), 2.18 (s, 6H), 1.35 (s, 9H).

### EXAMPLE 4(1)-4(18)

Using the compounds prepared in Example 1(2), 2(7), 3, 3(1), 3(2), 3(3), 3(4), 3(5), 3(6), 3(7), 3(8), 3(9), 3(10) and 3(11) instead of the compound prepared in Example 1(1), using corresponding imidate instead of N-(3-t-butyl-1-methyl-1H-pyrazol-5-yl)-4-(dimethyliminio)pyridine-1(4H)-carboxyimidate, the following compounds were obtained by the same procedure of Example 4.

### EXAMPLE 4(1)

### N-[3-t-butyl-1-(4-methylphenyl)-1H-pyrazol-5-yl]-N′-(4-{[3-(2-methylphenyl)-4-oxotetrahydropyrimidin-1(2H)-yl]methyl}phenyl)urea

TLC: Rf 0.23 (ethyl acetate);

NMR(CDCl₃): δ 7.32 (m, 2H), 7.23 (m, 9H), 7.02 (m, 2H), 6.54 (s, 1H), 6.35 (s, 1H), 4.18 (m, 2H), 3.82 (m, 2H), 3.09 (m, 2H), 2.53 (m, 2H), 2.37 (s, 3H), 2.19 (s, 3H), 1.35 (s, 9H).

### EXAMPLE 4(2)

### N-[3-t-butyl-1-(4-methylphenyl)-1H-pyrazol-5-yl]-N′-(4-{[3-(2,6-dimethylphenyl)-2,4-dioxotetrahydropyrimidin-1(2H)-yl]methyl}phenyl)urea

TLC: Rf 0.21 (hexane:ethyl acetate=1:1);

NMR(CDCl₃): δ 7.19 (m, 12H), 6.51 (m, 1H), 6.34 (s, 1H), 4.59 (m, 2H), 3.42 (m, 2H), 2.81 (m, 2H), 2.34 (m, 3H), 2.06 (m, 6H), 1.34 (s, 9H).

### EXAMPLE 4(3)

### N-[3-t-butyl-1-(4-methylphenyl)-1H-pyrazol-5-yl]-N′-(4-{[3-(2-methylphenyl)-2,4-dioxotetrahydro-1(2H)-pyrimidinyl]methyl}phenyl)urea

TLC: Rf 0.75 (ethyl acetate);

NMR(CDCl₃): δ 1.34 (s, 9H), 2.04 (m, J=1.28 Hz, 3H), 2.34 (s, 3H), 2.81 (t, J=6.77 Hz, 2H), 3.45 (t, J=6.77 Hz, 2H), 4.46 (d, J=14.7 Hz, 1H), 4.65 (d, J=14.7 Hz, 1H), 6.35 (s, 1H), 6.63 (s, 1H), 6.91 (d, J=7.51 Hz, 1H), 7.24 (m, 12H).

### EXAMPLE 4(4)

### N-[3-t-butyl-1-(4-methylphenyl)-1H-pyrazol-5-yl]-N′-(2-{[3-(2-methylphenyl)-2,4-dioxotetrahydro-1(2H)-pyrimidinyl]methyl}benzyl)urea

TLC: Rf 0.18 (n-hexane:ethyl acetate=1:1);

NMR(CDCl₃): δ 1.29 (s, 9H), 2.10 (s, 3H), 2.33 (s, 3H), 2.81 (t, J=6.77 Hz, 2H), 3.41 (m, 2H), 4.32 (dd, J=5.68, 2.20 Hz, 2H), 4.66 (m, 2H), 5.57 (t, J=5.68 Hz, 1H), 6.22 (s, 1H), 6.25 (s, 1H), 7.02 (d, J=7.32 Hz, 1H), 7.22 (m, 11H).

### EXAMPLE 4(5)

### N-(3-t-butyl-1-methyl-1H-pyrazol-5-yl)-N′-(2-{[3-(2-methylphenyl)-2,4-dioxotetrahydro-1(2H)-pyrimidinyl]methyl}benzyl)urea

TLC: Rf 0.25 (ethyl acetate); NMR(CDCl₃): δ 1.24 (s, 9H), 2.12 (s, 3H), 2.86 (t, J=6.77 Hz, 2H), 3.48 (m, 5H), 4.39 (d, J=5.68 Hz, 2H), 4.70 (m, 2H), 5.59 (t, J=5.86 Hz, 1H), 5.96 (s, 1H), 6.32 (s, 1H), 7.06 (d, J=7.32 Hz, 1H), 7.25 (m, 7H).

### EXAMPLE 4(6)

### N-[3-t-butyl-1-(4-methylphenyl)-1H-pyrazol-5-yl]-N′-{4-[(3-oxo-4-phenyl-1,4-diazepan-1-yl)methyl]phenyl}urea

TLC: Rf 0.32 (hexane:ethyl acetate=1:4);

NMR(CDCl₃): δ 1.33 (s, 9H), 1.92 (m, 2H), 2.36 (s, 3H), 2.99 (m, 2H), 3.43 (s, 2H), 3.73 (m, 4H), 6.29 (s, 1H), 6.92 (s, 1H), 6.99 (d, J=7.14 Hz, 2H), 7.21 (m, 11H), 7.72 (s, 1H).

### EXAMPLE 4(7)

### N-[3-t-butyl-1-(4-methylphenyl)-1H-pyrazol-5-yl]-N′-(4-{[3-(2-methylphenyl)-2,4-dioxo-1-imidazolidinyl]methyl}phenyl)urea

TLC: Rf 0.24 (ethyl acetate:hexane=1:1);

NMR(CDCl₃): δ 1.35 (s, 9H), 2.20 (s, 3H), 2.36 (s, 3H), 3.89 (m, 2H), 4.53 (d, J=15.01 Hz, 1H), 4.61 (d, J=15.01 Hz, 1H), 6.36 (s, 1H), 6.50 (s, 1H), 7.13 (m, 2H), 7.22 (m, 4H), 7.31 (m, 7H).

### EXAMPLE 4(8)

### N-[3-t-butyl-1-(4-methylphenyl)-1H-pyrazol-5-yl]-N′-(4-{[3-(2-methoxyphenyl)-2,4-dioxotetrahydro-1(2H)-pyrimidinyl]methyl}phenyl)urea

TLC: Rf 0.26 (n-hexane:ethyl acetate=1:2);

NMR(CDCl₃): δ 1.32 (s, 9H), 2.32 (s, 3H), 2.77 (t, J=6.77 Hz, 2H), 3.45 (m, 2H), 3.58 (s, 3H), 4.34 (d, J=14.83 Hz, 1H), 4.65 (d, J=14.83 Hz, 1H), 6.34 (s, 1H), 6.81 (m, 3H), 7.03 (s, 1H), 7.13 (m, 6H), 7.24 (m, 3H), 7.74 (s, 1H).

### EXAMPLE 4(9)

### N-[3-t-butyl-1-(4-methylphenyl)-1H-pyrazol-5-yl]-N′-(4-{[4-(2-methylphenyl)-5-oxo-1,4-diazepan-1-yl]methyl}phenyl)urea

TLC: Rf 0.34 (ethyl acetate:methanol=19:1);

NMR(CDCl₃): δ 1.35 (s, 9H), 2.21 (s, 3H), 2.37 (s, 3H), 2.74 (m, 6H), 3.59 (m, 3H), 3.81 (m, 1H), 6.35 (s, 1H), 6.43 (s, 1H), 6.92 (s, 1H), 7.04 (m, 1H), 7.20 (m, 9H), 7.33 (d, J=8.42 Hz, 2H).

### EXAMPLE 4(10)

### N-(3-t-butyl-5-isoxazolyl)-N′-(4-{[3-(2-methylphenyl)-2,4-dioxotetrahydro-1(2H)-pyrimidinyl]methyl}phenyl)urea

TLC: Rf 0.24 (hexane:ethyl acetate=1:2);

NMR(CDCl₃): δ 1.33 (s, 9H), 2.12 (s, 3H), 2.98 (m, 2H), 3.69 (m, 2H), 4.56 (d, J=15.01 Hz, 1H), 4.66 (d, J=15.01 Hz, 1H), 6.02 (s, 1H), 7.15 (m, 8H), 7.47 (s, 1H), 7.91 (s, 1H).

### EXAMPLE 4(11)

### N-(5-t-butyl-3-isoxazolyl)-N′-(4-{[3-(2-methylphenyl)-2,4-dioxotetrahydro-1(2H)-pyrimidinyl]methyl}phenyl)urea

TLC: Rf 0.35 (hexane:ethyl acetate=1:2);

NMR(CDCl₃): δ 1.36 (s, 9H), 2.14 (s, 3H), 2.85 (m, 2H), 3.44 (m, 2H), 4.55 (d, J=14.83 Hz, 1H), 4.72 (d, J=14.83 Hz, 1H), 5.94 (s, 1H), 7.11 (m, 1H), 7.28 (m, 5H), 7.49 (d, J=8.24 Hz, 2H), 8.30 (s, 1H), 9.16 (s, 1H).

### EXAMPLE 4(12)

### N-(3-t-butyl-1-methyl-1H-pyrazol-5-yl)-N′-(4-{[3-(2-methylphenyl)-2,4-dioxotetrahydro-1(2H)-pyrimidinyl]methyl}phenyl)urea

TLC: Rf 0.23 (hexane:ethyl acetate=1:9);

NMR(CDCl₃): δ 1.30 (s, 9H), 2.11 (s, 3H), 2.86 (m, 2H), 3.49 (m, 2H), 3.59 (s, 3H), 4.55 (d, J=14.83 Hz, 1H), 4.66 (d, J=14.83 Hz, 1H), 6.04 (s, 1H), 6.73 (m, 1H), 7.18 (m, 9H).

### EXAMPLE 4(13)

### methyl 2-[3-{4-[({[3-t-butyl-1-(4-methylphenyl)-1H-pyrazol-5-yl]amino}carbonyl)amino]benzyl}-2,6-dioxotetrahydro-1(2H)-pyrimidinyl]benzoate

TLC: Rf 0.25 (toluene:ethyl acetate=1:1);

NMR(CDCl₃): δ 1.35 (s, 9H), 2.36 (s, 3H), 2.81 (m, 2H), 3.52 (m, 2H), 3.70 (s, 3H), 4.36 (d, J=14.83 Hz, 1H), 4.77 (d, J=14.83 Hz, 1H), 6.33 (s, 1H), 6.66 (s, 1H), 7.01 (dd, J=7.69, 1.28 Hz, 1H), 7.23 (m, 9H), 7.39 (m, 1H), 7.48 (m, 1H), 8.05 (dd, J=7.87, 1.65 Hz, 1H).

### EXAMPLE 4(14)

### N-[3-t-butyl-1-(4-methylphenyl)-1H-pyrazol-5-yl]-N′-(4-{[4-(2-methylphenyl)-3-oxo-1-piperazinyl]methyl}phenyl)urea hydrochloride

TLC: Rf 0.24 (hexane:ethyl acetate=1:8);

NMR(CD₃OD): δ 1.41 (m, 9H), 2.24 (m, 3H), 2.50 (s, 3H), 3.75 (m, 2H), 4.08 (m, 4H), 4.50 (m, 2H), 7.27 (m, 5H), 7.55 (m, 8H).

### EXAMPLE 4(15)

### N-[3-t-butyl-1-(4-methylphenyl)-1H-pyrazol-5-yl]-N′-(3-{[4-(2-methylphenyl)-3-oxo-1-piperazinyl]methyl}phenyl)urea hydrochloride

TLC: Rf 0.21 (hexane:ethyl acetate=1:6);

NMR(CD₃OD): δ 1.43 (s, 9H), 2.24 (s, 3H), 2.50 (s, 3H), 3.74 (m, 2H), 4.08 (m, 4H), 4.53 (s, 2H), 6.88 (d, J=3.30 Hz, 1H), 7.28 (m, 5H), 7.51 (m, 6H), 7.83 (d, J=1.65 Hz, 1H).

### EXAMPLE 4(16)

### N-{4-[(3-benzyl-2,4-dioxotetrahydro-1(2H)-pyrimidinyl)methyl]phenyl}-N′-[3-t-butyl-1-(4-methylphenyl)-1H-pyrazol-5-yl]urea

TLC: Rf 0.54 (hexane:ethyl acetate=1:2);

NMR(CDCl₃): δ 1.32 (s, 9H), 2.31 (s, 3H), 2.65 (t, J=6.77 Hz, 2H), 3.25 (t, J=6.77 Hz, 2H), 4.48 (s, 2H), 4.91 (s, 2H), 6.37 (s, 1H), 6.88 (s, 1H), 7.11 (m, 6H), 7.27 (m, 7H), 7.40 (s, 1H).

### EXAMPLE 4(17)

### N-[3-t-butyl-1-(4-methylphenyl)-1H-pyrazol-5-yl]-N′-(4-{[3-(2-methylphenyl)-4-oxotetrahydro-1(2H)-pyrimidinyl]sulfonyl}phenyl)urea

TLC: Rf 0.47 (ethyl acetate);

NMR(CDCl₃): δ 1.34 (s, 9H), 2.11 (s, 3H), 2.29 (m, 2H), 2.36 (s, 3H), 3.65 (m, 2H), 4.79 (d, J=12.27 Hz, 1H), 4.91 (d, J=12.27 Hz, 1H), 6.39 (s, 1H), 7.00 (m, 1H), 7.22 (m, 10H), 7.68 (d, J=8.97 Hz, 2H), 7.96 (s, 1H).

### EXAMPLE 4(18)

### N-[3-t-butyl-1-(4-methylphenyl)-1H-pyrazol-5-yl]-N′-(4-{[4-(2-methylbenzoyl)-1-piperazinyl]methyl}phenyl)urea

TLC: Rf 0.15 (hexane:ethyl acetate=1:3);

NMR(CDCl₃): δ 1.32 (s, 9H), 2.26 (s, 3H), 2.30 (m, 5H), 2.42 (t, J=4.94 Hz, 2H), 3.20 (m, 2H), 3.44 (s, 2H), 3.69 (m, 2H), 6.35 (s, 1H), 6.87 (s, 1H), 7.13 (m, 1OH), 7.26 (m, 2H), 7.49 (s, 1H).

### EXAMPLE 5

### 3-(2,6-dichlorophenyl)-1-(2,4-difluorobenzoyl)tetrahydropyrimidin-4(1H)-one

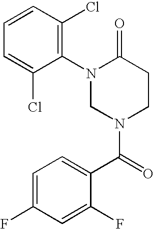

To a solution of the compound prepared in Reference Example 7 (100 mg) in dichloromethane (2 mL) were added pyridine (0.5 mL) and 2,4-difluorobenzoyl chloride(70 mg). The mixture was stirred for 30 minutes at room temperature. To the mixture were added water and ethyl acetate. The organic layer was washed with a saturated aqueous solution of sodium bicarbonate and a saturated aqueous solution of sodium chloride, subsequently, dried over an anhydrous magnesium sulfate and then concentrated. The residue was purified by column chromatography on silica gel (hexane:ethyl acetate=1:1) to give the compound of the present invention (100 mg) having the following physical data.

TLC: Rf 0.30 (hexane:ethyl acetate=1:1);

NMR(CDCl₃): δ 7.37 (m, 4H), 6.95 (m, 2H), 4.89-5.35 (m, 2H), 3.76-4.18 (m, 2H), 2.77-2.92 (m, 2H).

### EXAMPLE 5(1)-5(3)

Using the following compounds of (1)-(2) [(1) the compound which is obtained by the same procedures as a series of reactions of Reference Example 5→Reference Example 6→Reference Example 7, using (2-methylphenyl)amine instead of (4-bromo-2,6-dichlorophenyl)amine, and (2) the compound prepared in Example 1(2)] instead of the compound prepared in Reference Example 7, using corresponding acid chloride instead of 2,4-difluorobenzoyl chloride, the following compounds were obtained by the same procedure of Example 5.

### EXAMPLE 5(1)

### N-[3-t-butyl-1-(4-methylphenyl)-1H-pyrazol-5-yl]-N′-(4-{[3-(2-methylphenyl)-4-oxotetrahydro-1(2H)-pyrimidinyl]carbonyl}phenyl)urea

TLC: Rf 0.47 (ethyl acetate);

NMR(CDCl₃): δ 1.32 (s, 9H), 2.05 (s, 3H), 2.33 (s, 3H), 2.50 (m, 2H), 3.82 (m, 2H), 5.04 (m, 2H), 6.39 (s, 1H), 6.98 (s, 1H), 7.23 (m, 12H), 8.12 (s, 1H).

### EXAMPLE 5(2)

### N-(3-t-butyl-1-methyl-1H-pyrazol-5-yl)-N′-(4-{[3-(2-methylphenyl)-4-oxotetrahydro-1(2H)-pyrimidinyl]carbonyl}phenyl)urea

TLC: Rf 0.38 (ethyl acetate);

NMR(CDCl₃): δ 1.28 (s, 9H), 2.11 (m, 2H), 2.77 (m, 2H), 3.52 (s, 3H), 3.93 (m, 2H), 5.18 (m, 2H), 6.04 (s, 1H), 7.06 (m, 1H), 7.18 (m, 3H), 7.29 (m, 2H), 7.41 (m, 3H), 8.04 (s, 1H).

### EXAMPLE 5(3)

### N-(4-{[3-(2-methylphenyl)-4-oxotetrahydro-1(2H)-pyrimidinyl]methyl}phenyl)-2-(2-naphthyl)acetamide

TLC: Rf 0.38 (ethyl acetate);

NMR(CDCl₃): δ 2.20 (s, 3H), 2.61 (m, 2H), 3.10 (m, 2H), 3.82 (m, 2H), 3.90 (s, 2H), 4.11 (d, J=10.62 Hz, 1H), 4.24 (d, J=10.62 Hz, 1H), 7.04 (m, 1H), 7.11 (s, 1H), 7.22 (m, 5H), 7.37 (d, J=8.60 Hz, 2H), 7.44 (dd, J=8.42, 1.83 Hz, 1H), 7.53 (m, 2H), 7.86 (m, 4H).

### EXAMPLE 6

### N-(3,5-di-t-butylphenyl)-N′-(4-{[3-(2-methylphenyl)-2,4-dioxotetrahydro-1(2H)-pyrimidinyl]methyl}phenyl)urea

To a solution of (3,5-di-t-butylphenyl)amine (80 mg) in dichloromethane (1.0 mL) was added a solution of triethylamine (108 μL) and triphosgene (39 mg) in dichloromethane (0.5 mL) and the mixture was stirred for 10 minutes at room temperature. To the mixture was added a solution of the compound prepared in Example 3(1) (100 mg) in dichloromethane (1.0 mL). The mixture was stirred for 30 minutes at room temperature, and then added ethyl acetate. The mixture was washed with 1N hydrochloric acid, a saturated aqueous solution of sodium bicarbonate and a saturated aqueous solution of sodium chloride, subsequently, dried over an anhydrous magnesium sulfate and then concentrated to give the compound of the present invention (44 mg) having the following physical data.

TLC: Rf 0.63 (hexane:ethyl acetate=1:2);

NMR(CDCl₃): δ 1.32 (s, 18H), 2.13 (s, 3H), 2.85 (m, 2H), 3.47 (m, 2H), 4.55 (d, J=14.83 Hz, 1H), 4.68 (d, J=14.83 Hz, 1H), 6.55 (s, 1H), 6.85 (s, 1H), 7.08 (m, 1H), 7.15 (m, 2H), 7.27 (m, 8H).

### EXAMPLE 6(1)-6(6)

Using the compounds prepared in Example 1(2) instead of the compound prepared in Example 3(1), using corresponding amine instead of (3,5-di-t-butylphenyl)amine, the following compounds were obtained by the same procedure of Example 6.

### EXAMPLE 6(1)

### N-(3-t-butylphenyl)-N′-(4-{[3-(2-methylphenyl)-4-oxotetrahydro-1(2H)-pyrimidinyl]methyl}phenyl)urea

TLC: Rf 0.20 (ethyl acetate);

NMR(CDCl₃): δ 1.30 (s, 9H), 2.22 (s, 3H), 2.68 (m, 2H), 3.13 (t, J=6.50 Hz, 2H), 3.82 (m, 2H), 4.15 (d, J=10.80 Hz, 1H), 4.28 (d, J=10.80 Hz, 1H), 7.08 (m, 5H), 7.22 (m, 8H), 7.38 (t, J=1.92 Hz, 1H).

### EXAMPLE 6(2)

### N-(5-t-butyl-3-isoxazolyl)-N′-(4-{[3-(2-methylphenyl)-4-oxotetrahydro-1(2H)-pyrimidinyl]methyl}phenyl)urea

TLC: Rf 0.35 (ethyl acetate);

NMR(CDCl₃): δ 1.35 (s, 9H), 2.23 (s, 3H), 2.68 (m, 2H), 3.15 (t, J=6.41 Hz, 2H), 3.83 (d, J=13.36 Hz, 1H), 3.89 (d, J=13.36 Hz, 1H), 4.16 (d, J=10.80 Hz, 1H), 4.29 (d, J=10.80 Hz, 1H), 5.97 (s, 1H), 7.07 (m, 1H), 7.20 (m, 3H), 7.31 (d, J=8.24 Hz, 2H), 7.43 (d, J=8.24 Hz, 2H), 8.17 (s, 1H), 9.11 (s, 1H).

### EXAMPLE 6(3)

### N-(4-{[3-(2-methylphenyl)-4-oxotetrahydro-1(2H)-pyrimidinyl]methyl}phenyl)-N′-2-naphthylurea

TLC: Rf 0.56 (ethyl acetate);

NMR(DMSO-d₆): δ 2.14 (s, 3H), 2.46 (m, 2H), 3.07 (m, 2H), 3.85 (s, 2H), 4.09 (d, J=10.80 Hz, 1H), 4.34 (d, J=10.8 Hz, 1H), 7.23 (m, 7H), 7.46 (m, 4H), 7.80 (m, 3H), 8.09 (d, J=2.20 Hz, 1H), 8.72 (s, 1H), 8.85 (s, 1H).

### EXAMPLE 6(4)

### N-[3-t-butyl-1-(3-methylphenyl)-1H-pyrazol-5-yl]-N′-(4-{[3-(2-methylphenyl)-2,4-dioxotetrahydro-1(2H)-pyrimidinyl]methyl}phenyl)urea

TLC: Rf 0.30 (hexane:ethyl acetate=1:2);

NMR(CDCl₃): δ 1.35 (s, 9H), 2.04 (s, 3H), 2.36 (s, 3H), 2.83 (m, 2H), 3.46 (m, 2H), 4.46 (d, J=14.83 Hz, 1H), 4.65 (d, J=14.83 Hz, 1H), 6.35 (s, 1H), 6.58 (s, 1H), 6.92 (d, J=7.51 Hz, 1H), 7.21 (m, 12H).

### EXAMPLE 6(5)

### N-[3-t-butyl-1-(3-chlorophenyl)-1H-pyrazol-5-yl]-N′-(4-{[3-(2-methylphenyl)-2,4-dioxotetrahydro-1(2H)-pyrimidinyl]methyl}phenyl)urea

TLC: Rf 0.36 (hexane:ethyl acetate=1:2);

NMR(CDCl₃): δ 1.35 (s, 9H), 1.98 (s, 3H), 2.84 (m, 2H), 3.52 (m, 2H), 4.46 (d, J=14.83 Hz, 1H), 4.66 (d, J=14.83 Hz, 1H), 6.32 (s, 1H), 6.74 (s, 1H), 6.83 (d, J=7.69 Hz, 1H), 7.13 (m, 8H), 7.36 (m, 4H).

### EXAMPLE 6(6)

### N-[3-t-butyl-1-(2-pyridinyl)-1H-pyrazol-5-yl]-N′-(4-{[3-(2-methylphenyl)-2,4-dioxotetrahydro-1(2H)-pyrimidinyl]methyl}phenyl)urea

TLC: Rf 0.33 (hexane:ethyl acetate=1:2);

NMR(CDCl₃): δ 1.35 (s, 9H), 2.12 (s, 3H), 2.86 (m, 2H), 3.48 (m, 2H), 4.60 (d, J=14.83 Hz, 1H), 4.75 (d, J=14.83 Hz, 1H), 6.69 (s, 1H), 6.82 (s, 1H), 7.01 (m, 1H), 7.09 (m, 1H), 7.26 (m, 3H), 7.33 (d, J=8.24 Hz, 2H), 7.42 (d, J=8.24 Hz, 2H), 7.77 (m, 1H), 8.05 (m, 2H), 11.63 (s, 1H).

### EXAMPLE 7

### 6-(4-aminobenzyl)-2-(2-methylphenyl)-4,5-dihydro-3(2H)-pyridazinone

To a solution of 5-(4-aminophenyl)-4-oxopentanoic acid (151 mg) in acetic acid (5 mL) were added o-tolylhydrazine hydrochloride (98 mg) and sodium acetate (152 mg), and the mixture was stirred for 30 minutes at 110 degrees. The reaction mixture was concentrated and then added ethyl acetate and a saturated aqueous solution of sodium bicarbonate. The organic layer was dried over anhydrous sodium sulfate and concentrated to give the crude compound of the present invention (107 mg) having the following physical data.

TLC: Rf 0.57 (ethyl acetate);

NMR(CDCl₃): δ 2.17 (s, 2H), 2.24 (s, 2H), 2.52 (s, 3H), 3.55 (s, 2H), 6.68 (m, 3H), 7.03 (m, 2H), 7.27 (m, 3H).

### EXAMPLE 8

### N-[3-t-butyl-1-(4-methylphenyl)-1H-pyrazol-5-yl]-N′-(4-{[1-(2-methylphenyl)-6-oxo-1,4,5,6-tetrahydro-3-pyridazinyl]methyl}phenyl)urea

To a solution of the compound prepared in Example 7 (crude, 104 mg) in tetrahydrofuran (4 mL) was added N-(3-t-butyl-1-methyl-1H-pyrazol-5-yl)-4-(dimethyliminio)pyridine-1(4H)-carboxyimidate (133 mg) and the mixture was stirred for 15 minutes at room temperature. To the reaction mixture were added water and ethyl acetate. The organic layer was dried over anhydrous sodium sulfate, concentrated and purified by column chromatography on silica gel (hexane:ethyl acetate=2:3) to give the compound of the present invention (62 mg) having the following physical data.

TLC: Rf 0.42 (hexane:ethyl acetate=1:2);

NMR(CDCl₃): δ 1.35 (s, 9H), 2.22 (s, 3H), 2.36 (s, 3H), 2.48 (m, 4H), 3.62 (s, 2H), 6.35 (s, 1H), 6.46 (s, 1H), 6.97 (s, 1H), 7.25 (m, 12H).

### EXAMPLE 9(1)

### t-butyl 4-(3-methyl-4-nitrobenzyl)piperazine-1-carboxylate

To a solution of N-(t-butoxycarbonyl)piperazine (891 mg) in N,N-dimethylformamide (5 mL) were added potassium carbonate (782 mg) and 3-methyl-4-nitrobenzyl bromide (1 g) at 0 degree under an atmosphere of argon. The reaction mixture was allowed to raise to room temperature and then stirred for 1 hour. The reaction mixture was poured into ice and extracted with t-butyl methyl ether. The organic layer washed with a saturated aqueous solution of sodium chloride, dried over an anhydrous magnesium sulfate and concentrated. The obtained residue was purified by column chromatography on silica gel (hexane:ethyl acetate=2:1→1:1) to give the title compound (1.46 g) having the following physical data

TLC: Rf 0.50 (hexane:ethyl acetate=1:1);

NMR(CDCl₃): δ 7.95 (d, J=8.7 Hz, 1H), 7.34-7.28 (m, 2H), 3.52 (s, 2H), 3.44 (t, J=5.1 Hz, 4H), 2.60 (s, 3H), 2.38 (t, J=5.1 Hz, 4H), 1.45 (s, 9H).

### EXAMPLE 9(2)

### t-butyl 4-(4-chloro-3-nitrobenzyl)piperazine-1-carboxylate

Using 4-chloro-3-nitrobenzyl bromide instead of 3-methyl-4-nitrobenzyl bromide, the title compound having the following data was obtained by the same procedure of Example 9(1).

TLC: Rf 0.38 (hexane:ethyl acetate=1:1);

NMR(CDCl₃): δ 7.90-7.83 (m, 1H), 7.52-7.45 (m, 2H), 3.53 (s, 2H), 3.50-3.38 (m, 4H), 2.45-2.35 (m, 4H), 1.46 (s, 9H).

### Example 10(1)

### t-butyl 4-(4-amino-3-methylbenzyl)piperazine-1-carboxylate

To a mixture of the compound prepared in Example 9(1) (1.45 g), acetic acid (8 mL) and water (0.8 mL) was added iron powder (1.2 g). The mixture was stirred for 1 hour at 40 degrees. The reaction mixture was cooled with ice and added 5N aqueous solution of sodium hydroxide (50 mL). An insoluble material was removed and the filtrate was extracted with ethyl acetate. The organic layer was washed with a saturated aqueous solution of sodium chloride, dried over an anhydrous magnesium sulfate and concentrated to give the title compound (1.27 g) having the following physical data. The compound was used to next reaction without further purification.

TLC: Rf 0.12 (hexane:ethyl acetate=1:1);

NMR(CDCl₃): δ 7.00-6.92 (m, 2H), 6.62 (d, J=8.1 Hz, 1H), 3.57 (brs, 2H), 3.41 (t, J=5.1 Hz, 4H), 3.38 (s, 2H), 2.36 (t, J=5.1 Hz, 4H), 2.16 (s, 3H), 1.45 (s, 9H).

### EXAMPLE 10(2)

### t-butyl 4-(3-amino-4-chlorobenzyl)piperazine-1-carboxylate

Using the compound prepared in Example 9(2) instead of the compound prepared in Example 9(1), the title compound having the following data was obtained by the same procedure of Example 10(1).

TLC: Rf 0.25 (hexane:ethyl acetate=1:1);

NMR(CDCl₃): δ 7.17 (d, J=8.1 Hz, 1H), 6.76 (d, J=2.1 Hz, 1H), 6.63 (dd, J=8.1, 2.1 Hz, 1H), 4.02 (s, 2H), 3.45-3.35 (m, 4H), 3.38 (s, 2H), 2.42-2.30 (m, 4H), 1.45 (s, 9H).

### EXAMPLE 11(1)

### t-butyl 4-{4-[({[3-t-butyl-1-(4-methylphenyl)-1H-pyrazol-5-yl]amino}carbonyl)amino]-3-methylbenzyl}piperazine-1-carboxylate

To a solution of the compound prepared in Example 10(1) (1.25 g) in tetrahydrofuran (16 mL) was added N-[3-t-butyl-1-(4-methylphenyl)-1H-pyrazol-5-yl]-4-(dimethyliminio)pyridine-1(4H)-carboxyimidate (2.01 g), which was prepared by the procedure which improved one described in Angew. Chem. Int. Ed. Engl., 2497-2500, 34(22), 1995, at room temperature. The mixture was stirred for 1.5 hours. The reaction mixture was poured into ice-cooled aqueous solution of citric acid and extracted with ethyl acetate. The organic layer was washed with a saturated aqueous solution of sodium bicarbonate and a saturated aqueous solution of sodium chloride, subsequently, dried over an anhydrous magnesium sulfate and then concentrated. The obtained residue was purified by column chromatography on silica gel (hexane : ethyl acetate=1:1→1:3) to give the title compound (2.28 g) having the following physical data.

TLC: Rf 0.36 (hexane:ethyl acetate=1:3);

NMR(CDCl₃): δ 7.45 (d, J=8.4 Hz, 1H), 7.30 (d, J=8.1 Hz, 2H), 7.19 (d, J=8.1 Hz, 2H), 7.14-7.08 (m, 2H), 6.49 (s, 1H), 6.37 (s, 1H), 6.35 (s, 1H), 3.42 (s, 2H), 3.41 (t, J=5.1 Hz, 4H), 2.36 (s, 3H), 2.35 (t, J=5.1 Hz, 4H), 2.12 (s, 3H), 1.45 (s, 9H), 1.34 (s, 9H).

### EXAMPLE 11(2)

### t-butyl 4-{3-[({[3-t-butyl-1-(4-methylphenyl)-1H-pyrazol-5-yl]amino}carbonyl)amino]-4-chlorobenzyl}piperazine-1-carboxylate

Using the compound prepared in Example 10(2) instead of the compound prepared in Example 10(1), the title compound having the following data was obtained by the same procedure of Example 11(1).

TLC: Rf 0.30 (hexane:ethyl acetate=1:1);

NMR(CDCl₃): δ 8.15 (d, J=1.8 Hz, 1H), 7.53 (s, 1H), 7.36 (d, J=8.4 Hz, 2H), 7.30-7.20 (m, 3H), 7.00 (dd, J=8.4, 1.8 Hz, 1H), 6.42-6.38 (m, 2H), 3.46 (s, 2H), 3.45-3.35 (m, 4H), 2.42-2.32 (m, 7H), 1.44 (s, 9H), 1.36 (s, 9H).

### EXAMPLE 12(1)

### N-[3-t-butyl-1-(4-methylphenyl)-1H-pyrazol-5-yl]-N′-[2-methyl-4-(piperazin-1-ylmethyl)phenyl]urea dihydrochloride

To a solution of the compound prepared in Example 11(1) (2.27 g)in a mixture of ethyl acetate (15 mL) and methanol (10 mL) was added 4N hydrogen chloride in ethyl acetate (23 mL) at room temperature. The reaction mixture was concentrated. The obtained crude product was washed with mixed solvent of hexane:ethyl acetate=2:1 (80 mL) to give the title compound (2.16 g) having the following physical data. The compound was used to next reaction without further purification.

TLC: Rf 0.34 (dichloromethane:methanol:acetic acid=10:2:1);

NMR(CD₃OD): δ 8.01 (d, J=8.1 Hz, 1H), 7.60-7.50 (m, 4H), 7.49-7.38 (m, 2H), 6.95 (s, 1H), 4.42 (s, 2H), 3.70-3.40 (brs, 8H), 2.51 (s, 3H), 2.25 (s, 3H), 1.43 (s, 9H).

### EXAMPLE 12(2)

### N-[3-t-butyl-1-(4-methylphenyl)-1H-pyrazol-5-yl]-N′-[2-chloro-5-(piperazin-1-ylmethyl)phenyl]urea dihydrochloride

Using the compound prepared in Example 11(2) instead of the compound prepared in Example 11(1), the title compound having the following data was obtained by the same procedure of Example 12(1).

TLC: Rf 0.40 (ethyl acetate:acetic acid:water=3:1:1);

NMR(CD₃OD): δ 8.43-8.38 (m, 1H), 7.60-7.50 (m, 5H), 7.33 (dd, J=8.4, 2.1 Hz, 1H), 6.89 (s, 1H), 4.45 (s, 2H), 3.65-3.50 (m, 8H), 2.50 (s, 3H), 1.42 (s, 9H).

### EXAMPLE 13(1)

### N-[3-t-butyl-1-(4-methylphenyl)-1H-pyrazol-5-yl]-N′-{2-methyl-4-[(4-{4-[(4-methylpiperazin-1-yl)methyl]benzoyl}piperazin-1-yl)methyl]phenyl}urea

To a solution of the compound prepared in Example 12(1) (219 mg) in N,N-dimethylformamide (4 ml) were added 4-(N-methylpiperazinyl)methylbenzoic acid (96 mg), triethylamine (125 mg), 1-ethyl-3-(3-dimethylaminopropyl)carbodiimide hydrochloride (79 mg) and 1-hydroxybenzotriazole (55 mg) and the mixture was stirred for 5 hours at room temperature. To a reaction mixture was added a saturated aqueous solution of sodium bicarbonate. The mixture was extracted with ethyl acetate. The organic layer was washed with water, dried over anhydrous sodium sulfate and then concentrated. The obtained residue was purified by preparative thin layer chromatography (chloroform:methanol=5:1) to give the compound of the present invention (146 mg) having the following physical data. amorphous;

TLC: Rf 0.15 (chloroform:methanol=10:1);

NMR(CDCl₃): δ 1.34 (s, 9H), 2.10 (s, 3H), 2.28 (s, 3H), 2.34 (s, 3H), 2.24-2.67 (m, 12H), 3.33-3.49 (m, 4H), 3.51 (s, 2H), 3.59-3.90 (m, 2H), 6.36 (s, 1H), 6.51 (s, 1H), 6.62 (s, 1H), 7.07-7.13 (m, 2H), 7.18 (d, J=8.60 Hz, 2H), 7.28-7.39 (m, 6H), 7.50 (d, J=8.60 Hz, 1H).

### EXAMPLE 13(2)

### N-[3-t-butyl-1-(4-methylphenyl)-1H-pyrazol-5-yl]-N′-{2-chloro-5-[(4-{4-[(4-methylpiperazin-1-yl)methyl]benzoyl}piperazin-1-yl)methyl]phenyl}urea

Using the compound prepared in Example 12(2) instead of the compound prepared in Example 12(1), the compound of the present invention having the following data was obtained by the same procedure of Example 13(1).

TLC: Rf 0.50 (dichloromethane:methanol:ammonia water=90:10:1);

NMR(CD₃OD): δ 8.07 (d, J=1.8 Hz, 1H), 7.45-7.30 (m, 9H), 7.02 (dd, J=8.1, 1.8 Hz, 1H), 6.43 (s, 1H), 3.85-3.65 (m, 2H), 3.56 (s, 2H), 3.52 (s, 2H), 3.55-3.40 (m, 2H), 2.70-2.30 (m, 15H), 2.27 (s, 3H), 1.33 (s, 9H).

### EXAMPLE 14

### t-butyl 4-[(methylsulfonyl)oxy]piperidine-1-carboxylate

To a solution of N-Boc-4-hydroxypiperidine (2.00 g) in tetrahydrofuran (20 mL) were added triethylamine (2.28 mL) and methanesulfonyl chloride (1.26 mL) at 0 degree. The mixture was stirred for 1 hour. To the mixture was added ethyl acetate. The mixture was washed with a saturated aqueous solution of sodium bicarbonate and a saturated aqueous solution of sodium chloride, subsequently, dried over an anhydrous magnesium sulfate and concentrated. The obtained residue was solidified by adding hexane. The solid was washed and collected by suction to give the title compound (2.62 g) having the following physical data.

TLC: Rf 0.45 (ethyl acetate:hexane=1:1);

NMR(CDCl₃): δ 1.46 (s, 9H), 1.73-1.87 (m, 2H), 1.89-2.05 (m, 2H), 3.04 (s, 3H), 3.19-3.42 (m, 2H), 3.58-3.82 (m, 2H), 4.89 (m, 1H).

### EXAMPLE 15

### t-butyl 4-(3-methyl-4-nitrophenoxy)piperidine-1-carboxylate

To a solution of 3-methyl-4-nitrophenol (2.07 g) in anhydrous N,N-dimethylformamide (10 mL) was added sodium hydride (60%,540 mg) at 0 degree. The mixture was stirred for 15 minutes at room temperature.

To the mixture was added a solution of the compound prepared in Example 14 (1.89 g) in anhydrous N,N-dimethylformamide (2 mL). The mixture was stirred for 4 hours at 100 degrees. After cooling to room temperature, to the mixture was added water. The mixture was extracted with t-butyl methyl ether. The organic layer was washed with 1N aqueous solution of sodium hydroxide, water and a saturated aqueous solution of sodium chloride, subsequently, dried over an anhydrous magnesium sulfate and then concentrated. The obtained residue was solidified by adding isopropylether/hexane. The solid was washed and collected by suction to give the title compound (1.35 g) having the following physical data.

TLC: Rf 0.28 (ethyl acetate:hexane=3:7);

NMR(CDCl₃): δ 1.45 (s, 9H) 1.71-1.85 (m, 2H) 1.87-2.01 (m, 2H) 2.62 (s, 3H) 3.34-3.44 (m, 2H) 3.63-3.74 (m, 2H) 4.57 (m, 1H) 6.77-6.82 (m, 2H) 8.07 (m, 1H).

### EXAMPLE 16

### t-butyl 4-{4-[({[3-t-butyl-1-(4-methylphenyl)-1H-pyrazol-5-yl]amino}carbonyl)amino]-3-methylphenoxy}piperidine-1-carboxylate

To a solution of the compound prepared in Example 15 (1.34 g) in a mixture of acetic acid (10 mL) and water (1 mL) was added iron powder (1.02 g). The mixture was stirred for 2 hours at 70 degrees. To the mixture was added ethyl acetate. The mixture was filtered through Celite/Florisil silicagel to remove an insoluble material. The filtrate was washed with a saturated aqueous solution of sodium bicarbonate and a saturated aqueous solution of sodium chloride, subsequently, dried over an anhydrous magnesium sulfate and then concentrated to give crude product. To a solution of the crude product in tetrahydrofuran (20 mL) was added N-[3-t-butyl-1-(4-methylphenyl)-1H-pyrazol-5-yl]-4-(dimethyliminio)pyridine-1(4H)-carboxyimidate (1.79 g). The mixture was stirred for 30 minutes at room temperature. The reaction mixture was concentrated. The residue was purified by column chromatography on silica gel (ethyl acetate:hexane=3:7) to give the title compound (1.98 g) having the following physical data. amorphous powder;

TLC: Rf 0.32 (ethyl acetate:hexane=2:3);

NMR(CDCl₃): δ 1.33 (s, 9H), 1.44 (s, 9H), 1.63-1.78 (m, 2H), 1.82-1.96 (m, 2H), 2.10 (s, 3H), 2.36 (s, 3H), 3.27-3.38 (m, 2H), 3.62-3.74 (m, 2H), 4.40 (m, 1H), 6.29 (s, 1H), 6.38 (s, 1H), 6.42 (s, 1H), 6.63-6.74 (m, 2H), 7.12-7.26 (m, 5H).

### EXAMPLE 17

### N-[3-t-butyl-1-(4-methylphenyl)-1H-pyrazol-5-yl]-N′-{2-methyl-4-[(1-{4-[(4-methylpiperazin-1-yl)methyl]benzoyl}piperidin-4-yl)oxy]phenyl}urea

To a solution of the compound prepared in Example 16 (1.98 g) in ethyl acetate (2 mL) was added 4N hydrogen chloride in ethyl acetate (10 mL) and the mixture was stirred for 3 hours at room temperature. An appeared powder was collected by suction to give crude product. To the solution of crude product (131 mg) in N,N-dimethylformamide (5 mL) were added 4-(N-methylpiperazinyl)methylbenzoic acid (61 mg), 1-hydroxybenzotriazole (56 mg), 1-ethyl-3-(3-dimethylaminopropyl)carbodiimide hydrochloride (143 mg) and triethylamine (108 μL). The mixture was stirred for 2 hours at room temperature. To the mixture was added ethyl acetate. The mixture was washed with a saturated aqueous solution of sodium bicarbonate and a saturated aqueous solution of sodium chloride, subsequently, dried over an anhydrous magnesium sulfate and then concentrated. The obtained residue was purified by preparative thin layer chromatography (dichloromethane:methanol:ammonia water=90:10:1) to give the compound of the present invention (98 mg) having the following physical data.

amorphous powder;

TLC: Rf 0.22 (dichloromethane:methanol:ammonia water=90:10:1);

NMR(CDCl₃): δ 1.34 (s, 9H), 1.64-2.09 (m, 4H), 2.12 (s, 3H), 2.31 (s, 3H), 2.34 (s, 3H), 2.38-2.62 (m, 8H), 3.28-3.51 (m, 1H), 3.53 (s, 2H), 3.56-4.02 (m, 3H), 4.43-4.63 (m, 1H), 6.32 (s, 1H), 6.37 (s, 1H), 6.39 (s, 1H), 6.61-6.82 (m, 2H), 7.16 (d, J=8.24 Hz, 2H), 7.22-7.31 (m, 3H), 7.33-7.39 (m, 4H).

### EXAMPLE 18(1)-(21)

Using corresponding bromide instead of 3-methyl-4-nitrobenzyl bromide, using corresponding carboxylic acid instead of 4-(N-methylpiperazinyl)methylbenzoic acid, the compounds of the present invention having the following physical data were obtained by the same procedures as a series of reactions of Example 9(1)→Example 10(1)→Example 11( 1)→Example 12(1)→Example 13(1).

### EXAMPLE 18(1)

### N-[3-t-butyl-1-(4-methylphenyl)-1H-pyrazol-5-yl]-N′-(3-[4-(2-methylbenzoyl)-1-piperazinyl]methylphenyl)urea

TLC: Rf 0.36 (ethyl acetate);

NMR(CDCl₃): δ 1.33 (s, 9H), 2.29 (m, 8H), 2.45 (m, 2H), 3.19 (m, 2H), 3.44 (s, 2H), 3.73 (m, 2H), 6.36 (s, 1H), 6.79 (s, 1H), 6.98 (m, 1H), 7.08 (m, 1H), 7.19 (m, 7H), 7.28 ( m, 3H), 7.40 (s, 1H).

### EXAMPLE 18(2)

### N-[3-t-butyl-1-(4-methylphenyl)-1H-pyrazol-5-yl]-N′-(2-methyl-4-[4-(2-methylbenzoyl)-1-piperazinyl]methylphenyl)urea

TLC: Rf 0.43 (dichloromethane:methanol=10:1);

NMR(CD₃OD): δ 7.51 (d, J=8.1 Hz, 1H), 7.38-7.20 (m, 7H), 7.18-7.08 (m, 3H), 6.38 (s, 1H), 3.84-3.74 (m,2H), 3.48 (s, 2H), 3.28-3.22 (m, 2H), 2.53 (t, J=5.1 Hz, 2H), 2.42 (s, 3H), 2.36 (t, J=5.1 Hz, 2H), 2.27 (s, 3H), 2.16 (s, 3H), 1.32 (s, 9H).

### EXAMPLE 18(3)

### N-[3-t-butyl-1-(4-methylphenyl)-1H-pyrazol-5-yl]-N′-(3-chloro-4-[4-(2-methylbenzoyl)-1-piperazinyl]methylphenyl)urea

TLC: Rf 0.51 (dichloromethane:methanol=10:1);

NMR(CD₃OD): δ 7.55 (d, J=2.4 Hz, 1H), 7.40-7.13 (m, 10H), 6.40 (s, 1H), 3.82-3.75 (m, 2H), 3.60 (s, 2H), 3.28-3.22 (m, 2H), 2.59 (t, J=5.4 Hz, 2H), 2.46-2.38 (m, 2H), 2.41 (s, 3H), 2.27 (s, 3H), 1.32 (s, 9H).

### EXAMPLE 18(4)

### N-[3-t-butyl-1-(4-methylphenyl)-1H-pyrazol-5-yl]-N′-[4-(4-[3-(dimethylamino)benzoyl]-1-piperazinylmethyl)-2-methylphenyl]urea dihydrochloride

TLC: Rf 0.12 (ethyl acetate);

NMR(DMSO-d₆): δ 1.26 (s, 9H), 2.20 (s, 3H), 2.35 (s, 3H), 3.10 (m, 14H), 4.21 (m, 2H), 6.31 (s, 1H), 7.05 (s, 1H), 7.37 (m, 8H), 7.84 (d, J=8.24 Hz, 1H), 8.62 (s, 1H), 9.17 (s, 1H), 11.28 (s, 1H).

### EXAMPLE 18(5)

### N-[3-t-butyl-1-(4-methylphenyl)-1H-pyrazol-5-yl]-N′-4-[(4-4-[(E)-(hydroxyimino)methyl]benzoyl-1-piperazinyl)methyl]-2-methylphenylurea

TLC: Rf 0.27 (chloroform:methanol=10:1);

NMR(CDCl₃): δ 1.34 (s, 9H), 2.09 (s, 3H), 2.41 (m, 7H), 3.42 (m, 4H), 3.75 (m, 2H), 6.36 (s, 1H), 6.51 (s, 1H), 6.66 (s, 1H), 7.10 (m, 2H), 7.18 (d, J=8.06 Hz, 2H), 7.31 (m, 2H), 7.37 (d, J=8.24 Hz, 2H), 7.49 (d, J=8.79 Hz, 1H), 7.57 (d, J=8.24 Hz, 2H), 8.11 (s, 1H), 8.21 (s, 1H).

### EXAMPLE 18(6)

### N-[3-t-butyl-1-(4-methylphenyl)-1H-pyrazol-5-yl]-N′-4-[(4-4-[(dimethylamino)methyl]benzoyl-1-piperazinyl)methyl]-2-methylphenylurea

TLC: Rf 0.32 (dichloromethane:methanol:ammonia water=9:1:0.1);

NMR(CDCl₃): δ 1.34 (s, 9H), 2.11 (s, 3H), 2.23 (s, 6H), 2.40 (m, 7H), 3.44 (m, 6H), 3.73 (m, 2H), 6.36 (s, 1H), 6.43 (s, 1H), 6.58 (s, 1H), 7.09 (m, 2H), 7.18 (d, J=8.06 Hz, 2H), 7.31 (m, 6H), 7.49 (m, 1H).

### EXAMPLE 18(7)

### N-[3-t-butyl-1-(4-methylphenyl)-1H-pyrazol-5-yl]-N′-(2,6-dimethyl-4-[4-(2-methylbenzoyl)-1-piperazinyl]methylphenyl)urea

TLC: Rf 0.52 (dichloromethane:methanol=10:1);

NMR(CD₃OD): δ 7.45-7.20 (m, 7H), 7.15 (m, 1H), 7.03 (s, 2H), 6.31 (s, 1H), 3.90-3.70 (m, 2H), 3.46 (s, 2H), 3.25-3.20 (m, 2H), 2.53 (t, J=5.1 Hz, 2H), 2.41 (s, 3H), 2.36 (t, J=5.1 Hz, 2H), 2.27 (s, 3H), 2.14 (s, 6H), 1.32 (s, 9H).

### EXAMPLE 18(8)

### N-[3-t-butyl-1-(4-methylphenyl)-1H-pyrazol-5-yl]-N′-(2-fluoro-4-[4-(2-methylbenzoyl)-1-piperazinyl]methylphenyl)urea

TLC: Rf 0.25 (hexane:ethyl acetate=1:2);

NMR(CD₃OD): δ 7.97 (t, J=8.7 Hz, 1H), 7.40-7.20 (m, 7H), 7.20-7.03 (m, 3H), 6.42 (s, 1H), 3.86-3.72 (m, 2H), 3.50 (s, 2H), 3.34-3.20 (m, 2H), 2.53 (t, J=5.1 Hz, 2H), 2.42 (s, 3H), 2.36 (t, J=5.1 Hz, 2H), 2.27 (s, 3H), 1.32 (s, 9H).

### EXAMPLE 18(9)

### N-[3-t-butyl-1-(4-methylphenyl)-1H-pyrazol-5-yl]-N′-(2-methyl-5-[4-(2-methylbenzoyl)-1-piperazinyl]methylphenyl)urea

TLC: Rf 0.16 (hexane:ethyl acetate=1:4);

NMR(CD₃OD): δ 7.55 (d, J=1.5 Hz, 1H), 7.40-7.20 (m, 7H), 7.20-7.05 (m, 2H), 7.00 (dd, J=7.5, 1.5 Hz, 1H), 6.39 (s, 1H), 3.90-3.70 (m, 2H), 3.50 (s, 2H), 3.30-3.20 (m, 2H), 2.54 (t, J=5.1 Hz, 2H), 2.42 (s, 3H), 2.37 (t, J=5.1 Hz, 2H), 2.27 (s, 3H), 2.15 (s, 3H), 1.32 (s, 9H).

### EXAMPLE 18(10)

### N-[3-t-butyl-1-(4-methylphenyl)-1H-pyrazol-5-yl]-N′-4-[(4-4-[(dimethylamino)methyl]benzoyl-1-piperazinyl)methyl]-2-fluorophenylurea

TLC: Rf 0.30 (dichloromethane:methanol:ammonia water=9:1:0.1);

NMR(CDCl₃): δ 1.35 (s, 9H), 2.25 (s, 6H), 2.45 (m, 7H), 3.47 (m, 6H), 3.74 (m, 2H), 6.36 (s, 1H), 6.60 (s, 1H), 7.05 (m, 2H), 7.25 (m, 3H), 7.32 (m, 6H), 8.03 (t, J=8.42 Hz, 1H).

### EXAMPLE 18(11)

### N-[3-t-butyl-1-(4-methylphenyl)-1H-pyrazol-5-yl]-N′-2-fluoro-4-[(4-4-[(4-methyl-1-piperazinyl)methyl]benzoyl-1-piperazinyl)methyl]phenylurea

TLC: Rf 0.24 (dichloromethane:methanol:ammonia water=9:1:0.1);

NMR(CDCl₃): δ 1.35 (s, 9H), 2.30 (s, 3H), 2.41 (m, 15H), 3.41 (m, 4H), 3.51 (s, 2H), 3.72 (m, 2H), 6.36 (s, 1H), 6.86 (s, 1H), 7.03 (m, 2H), 7.20 (d, J=8.06 Hz, 2H), 7.30 (m, 6H), 7.42 (m, 1H), 8.03 (t, J=8.42 Hz, 1H).

### EXAMPLE 18(12)

### N-[3-t-butyl-1-(4-methylphenyl)-1H-pyrazol-5-yl]-N′-3-[(4-4-[(dimethylamino)methyl]benzoyl-1-piperazinyl)methyl]-2-methylphenylurea

TLC: Rf 0.29 (chloroform:methanol=9:1);

NMR(CDCl₃): δ 1.34 (s, 9H), 2.14 (s, 3H), 2.24 (s, 6H), 2.39 (m, 7H), 3.37 (m, 6H), 3.70 (m, 2H), 6.39 (s, 1H), 6.44 (s, 1H), 6.56 (s, 1H), 7.08 (m, 1H), 7.14 (m, 3H), 7.26 (m, 2H), 7.34 (m, 4H), 7.39 (dd, J=7.32, 1.65 Hz, 1H).

### EXAMPLE 18(13)

### N-[3-t-butyl-1-(4-methylphenyl)-1H-pyrazol-5-yl]-N′-2-methyl-3-[(4-4-[(4-methyl-1-piperazinyl)methyl]benzoyl-1-piperazinyl)methyl]phenylurea

TLC: Rf 0.22 (chloroform:methanol=9:1);

NMR(CDCl₃): δ 1.34 (s, 9H), 2.14 (s, 3H), 2.29 (s, 3H), 2.33 (s, 3H), 2.46 (m, 12H), 3.35 (m, 4H), 3.51 (s, 2H), 3.70 (m, 2H), 6.39 (s, 1H), 6.43 (s, 1H), 6.56 (s, 1H), 7.08 (m, 2H), 7.14 (m, 2H), 7.26 (m, 2H), 7.33 (m, 4H), 7.39 (dd, J=7.41, 1.56 Hz, 1H).

### EXAMPLE 18(14)

### N-[3-t-butyl-1-(4-methylphenyl)-1H-pyrazol-5-yl]-N′-5-[(4-4-[(dimethylamino)methyl]benzoyl-1-piperazinyl)methyl]-2-methylphenylurea

TLC: Rf 0.14 (chloroform:methanol=9:1);

NMR(CDCl₃): δ 1.34 (s, 9H), 2.10 (s, 3H), 2.24 (s, 6H), 2.37 (m, 7H), 3.41 (m, 6H), 3.73 (m, 2H), 6.37 (s, 1H), 6.47 (s, 1H), 6.64 (s, 1H), 7.02 (dd, J=7.69, 1.65 Hz, 1H), 7.11 (d, J=7.69 Hz, 1H), 7.16 (d, J=8.06 Hz, 2H), 7.29 (d, J=8.06 Hz, 2H), 7.33 (m, 4H), 7.53 (s, 1H).

### EXAMPLE 18(15)

### N-[3-t-butyl-1-(4-methylphenyl)-1H-pyrazol-5-yl]-N′-2-methyl-5-[(4-4-[(4-methyl-1-piperazinyl)methyl]benzoyl-1-piperazinyl)methyl]phenylurea

TLC: Rf 0.16 (chloroform:methanol=9:1);

NMR(CDCl₃): δ 1.34 (s, 9H), 2.10 (s, 3H), 2.37 (m, 18H), 3.38 (m, 4H), 3.51 (s, 2H), 3.72 (m, 2H), 6.37 (s, 1H), 6.46 (s, 1H), 6.63 (s, 1H), 7.03 (dd, J=7.87, 1.46 Hz, 1H), 7.11 (d, J=7.87 Hz, 1H), 7.17 (d, J=8.24 Hz, 2H), 7.32 (m, 6H), 7.54 (s, 1H).

### EXAMPLE 18(16)

### N-[3-t-butyl-1-(4-methylphenyl)-1H-pyrazol-5-yl]-N′-(2-fluoro-5-[4-(2-methylbenzoyl)-1-piperazinyl]methylphenyl)urea

TLC: Rf 0.22 (hexane:ethyl acetate=1:4);

NMR(CD₃OD): δ 1.33 (s, 9H), 2.27 (s, 3H), 2.32-2.46 (m, 5H), 2.51-2.59 (m, 2H), 3.22-3.34 (m, 2H), 3.51 (s, 2H), 3.72-3.88 (m, 2H), 6.43 (s, 1H), 6.94-7.10 (m, 2H), 7.10-7.43 (m, 8H), 8.06 (dd, J=7.78, 1.56 Hz, 1H).

### EXAMPLE 18(17)

### N-[3-t-butyl-1-(4-methylphenyl)-1H-pyrazol-5-yl]-N′-(2-chloro-5-[4-(2-methylbenzoyl)-1-piperazinyl]methylphenyl)urea

TLC: Rf 0.52 (ethyl acetate);

NMR(CD₃OD): δ 1.33 (s, 9H), 2.27 (s, 3H), 2.33-2.46 (m, 5H), 2.50-2.60 (m, 2H), 3.22-3.33 (m, 2H), 3.52 (s, 2H), 3.73-3.88 (m, 2H), 6.42 (s, 1H), 7.02 (dd, J=8.24, 1.46 Hz, 1H), 7.11-7.43 (m, 9H), 8.06 (d, J=1.46 Hz, 1H).

### EXAMPLE 18(18)

### N-[3-t-butyl-1-(4-methylphenyl)-1H-pyrazol-5-yl]-N′-(2-methoxy-5-[4-(2-methylbenzoyl)-1-piperazinyl]methylphenyl)urea

TLC: Rf 0.23 (ethyl acetate);

NMR(CD₃OD): δ 8.05 (d, J=2.1 Hz, 1H), 7.40-7.10 (m, 8H), 6.95 (dd, J=8.4, 2.1 Hz, 1H), 6.89 (dd, J=8.4 Hz, 1H), 6.41 (s, 1H), 3.90-3.70 (m, 2H), 3.82 (s, 3H), 3.31 (s, 2H), 3.30-3.20 (m, 2H), 2.54 (t, J=5.1 Hz, 2H), 2.41 (s, 3H), 2.38 (t, J=5.1 Hz, 2H), 2.27 (s, 3H), 1.33 (s, 9H).

### EXAMPLE 18(19)

### N-[3-t-butyl-1-(4-methylphenyl)-1H-pyrazol-5-yl]-N′-2-fluoro-5-[(4-4-[(4-methyl-1-piperazinyl)methyl]benzoyl-1-piperazinyl)methyl]phenylurea

TLC: Rf 0.22 (dichloromethane:methanol:ammonia water=9:1:0.1);

NMR(CDCl₃): δ 1.35 (s, 9H), 2.30 (s, 3H), 2.32-2.59 (m, 15H), 3.33-3.56 (m, 6H), 3.58-3.85 (m, 2H), 6.37 (s, 1H), 6.81-6.89 (m, 1H), 6.92-7.07 (m, 2H), 7.21 (d, J=8.06 Hz, 2H), 7.25-7.39 (m, 6H), 7.40-7.48 (m, 1H), 8.06-8.15 (m, 1H).

### EXAMPLE 18(20)

### N-[3-t-butyl-1-(4-methylphenyl)-1H-pyrazol-5-yl]-N′-2-methyl-4-[(4-3-[(4-methyl-1-piperazinyl)methyl]benzoyl-1-piperazinyl)methyl]phenylurea

TLC: Rf 0.19 (dichloromethane:methanol:ammonia water=9:1:0.1);

NMR(CDCl₃): δ 1.34 (s, 9H), 2.11 (s, 3H), 2.30 (s, 3H), 2.33-2.65 (m, 15H), 3.30-3.47 (m, 4H), 3.52 (s, 2H), 3.64-3.91 (m, 2H), 6.36 (s, 1H), 6.42 (s, 1H), 6.59 (s, 1H), 7.05-7.14 (m, 2H), 7.19 (d, J=8.60 Hz, 2H), 7.23-7.42 (m, 6H), 7.50 (d, J=8.60 Hz, 1H).

### EXAMPLE 18(21)

### N-[3-t-butyl-1-(4-methylphenyl)-1H-pyrazol-5-yl]-N′-2-methyl-4-[(4-2-[(4-methyl-1-piperazinyl)methyl]benzoyl-1-piperazinyl)methyl]phenylurea

TLC: Rf 0.45 (dichloromethane:methanol:ammonia water=90:10:1);

NMR(CD₃OD): δ 1.32 (s, 9H), 2.17 (s, 3H), 2.21-2.78 (m, 18H), 3.14-3.40 (m, 3H), 3.38-3.60 (m, 3H), 3.76 (d, J=13.18 Hz, 1H), 3.90-4.08 (m, 1H), 6.38 (s, 1H), 7.06-7.25 (m, 3H), 7.26-7.46 (m, 7H), 7.53 (d, J=8.06 Hz, 1H).

### EXAMPLE 19

### N-4-[(1-benzoyl-4-piperidinyl)oxy]-2-methylphenyl-N′-[3-t-butyl-1-(4-methylphenyl)-1H-pyrazol-5 -yl]urea

Using benzoic acid instead of 4-(N-methylpiperazinyl)methylbenzoic acid, the title compound having the following physical data were obtained by the same procedures as a series of reactions of Example 14→Example 15→Example 16→Example 17.

TLC: Rf 0.36 (ethyl acetate:hexane=7:3);

NMR(CDCl₃): δ 1.33 (s, 9H), 1.66-2.08 (m, 4H), 2.10 (s, 3H), 2.33 (s, 3H), 3.19-3.53 (m, 1H), 3.54-4.06 (m, 3H), 4.36-4.66 (m, 1H), 6.37 (s, 2H), 6.46 (s, 1H), 6.63-6.80 (m, 2H), 7.15 (d, J=8.06 Hz, 2H), 7.20-7.31 (m, 3H), 7.36-7.48 (m, 5H).

### EXAMPLE 20

### N-[3-t-butyl-1-(4-methylphenyl)-1H-pyrazol-5-yl]-N′-(4-nitrophenyl)urea

To a solution of 4-nitroaniline (1.51 g) in tetrahydrofuran (55 mL) was added N-[3-t-butyl-1-(4-methylphenyl)-1H-pyrazol-5-yl]-4-(dimethyliminio)pyridine-1(4H)-carboxyimidate (4.74 g) and the mixture was stirred for 5.5 hours at room temperature. The reaction mixture was poured into water. The mixture was extracted with t-butyl methyl ether. The obtained organic layer was washed with diluted hydrochloric acid, water and a saturated aqueous solution of sodium chloride, subsequently, dried and concentrated to give the compound of the present invention (4.80 g) having the following physical data.

TLC: Rf 0.55 (ethyl acetate:hexane=3:2);

NMR(CDCl₃): δ 1.23 (s, 9H), 2.31 (s, 3H), 6.47 (s, 1H), 6.85-6.98 (m, 1H), 7.00-7.11 (m, 2H), 7.12-7.23 (m, 2H), 7.53 (d, J=9.15 Hz, 2H), 8.16 (d, J=9.15 Hz, 2H), 8.31-8.68 (m, 1H).

### EXAMPLE 21

### N-(4-aminophenyl)-N′-[3-t-butyl-1-(4-methylphenyl)-1H-pyrazol-5-yl]urea

To a solution of the compound prepared in Example 20 (1.00 g) in ethyl acetate (12 mL) was added 5% Pd—C (55% H₂O, 50 mg). The mixture was stirred vigorously for 2.5 hours at room temperature under an atmosphere of hydrogen. The catalyst was removed by filtration using Celite. The filtrate was concentrated to give the compound of the present invention (0.87 g) having the following physical data

TLC: Rf 0.52 (ethyl acetate);

NMR(CDCl₃): δ 1.32 (s, 9H), 2.30-2.38 (m, 3H), 3.57-3.72 (m, 2H), 6.33-6.38 (m, 1H), 6.42-6.53 (m, 2H), 6.54-6.62 (m, 2H), 6.91-6.99 (m, 2H), 7.13-7.26 (m, 4H).

### EXAMPLE 22

### benzyl 4-({4-[({[3-t-butyl-1-(4-methylphenyl)-1H-pyrazol-5-yl]amino}carbonyl)amino]phenyl}amino)piperidine-1-carboxylate

To a solution of the compound prepared in Example 21 (400 mg) in 1,2-dichloroethane (3.0 mL) were added benzyl 4-oxopiperidine-1-carboxylate (282 mg) and acetic acid (0.076 mL), and the mixture was stirred. To the mixture was added sodium triacetoxyborohydride (327 mg). The mixture was stirred for 30 minutes at room temperature. To the reaction mixture was added a saturated aqueous solution of sodium bicarbonate. After stirring vigorously, the mixture was extracted with ethyl acetate. The organic layer was washed with water and a saturated aqueous solution of sodium chloride, subsequently, dried and concentrated. The obtained residue was purified by column chromatography on silica gel (ethyl acetate:hexane=1:1→4:1) to give the compound of the present invention (411 mg) having the following physical data.

TLC: Rf 0.67 (ethyl acetate);

NMR(CDCl₃): δ 1.30-1.41 (m, 11H), 1.98-2.10 (m, 2H), 2.34 (s, 3H) 2.91-3.09 (m, 2H), 3.31-3.46 (m, 1H), 3.48-3.60 (m, 1H), 4.05-4.21 (m, 2H), 5.14 (s, 2H), 6.31-6.36 (m, 1H), 6.37 (s, 2H) 6.51 (d, J=8.79 Hz, 2H), 7.00 (d, J=8.79 Hz, 2H) 7.13-7.20 (m, 2H), 7.22-7.29 (m, 2H), 7.31-7.40 (m, 5H).

### EXAMPLE 23

### N-[3-t-butyl-1-(4-methylphenyl)-1H-pyrazol-5-yl]-N′-[4-(piperidin-4-ylamino)phenyl]urea

To a solution of the compound prepared in Example 22 (410 mg) in ethyl acetate (7.0 mL) were added 2N hydrochloric acid (0.1 mL), methanol (3.5 mL) and 5% Pd—C (55% H₂O, 40 mg). The mixture was stirred vigorously for 2 hours at room temperature under an atmosphere of hydrogen. The catalyst was removed by filtration using Celite. The filtrate was concentrated to give the compound of the present invention (309 mg) having the following physical data.

TLC: Rf 0.14 (ethyl acetate:methanol:triethylamine=3:6:1);

NMR(DMSO-d₆): δ 1.25 (s, 9H), 1.27-1.38 (m, 2H), 1.83-1.96 (m, 2H), 2.36 (s, 3H), 2.61-2.76 (m, 2H), 3.00-3.12 (m, 2H), 3.19-3.43 (m, 2H), 5.22 (d, J=8.06 Hz, 1H), 6.29 (s, 1H), 6.50 (d, J=9.03 Hz, 2H), 7.06 (d, J=9.03 Hz, 2H), 7.27-7.41 (m, 4H), 8.15 (s, 1H), 8.55 (s, 1H).

### EXAMPLE 24

### N-4-[(1-benzoylpiperidin-4-yl)amino]phenyl-N′-[3-t-butyl-1-(4-methylphenyl)-1H-pyrazol-5-yl]urea

To a solution of the compound prepared in Example 23 (292 mg) in dichloromethane (3.0 mL) was added triethylamine (0.18 mL). The mixture was stirred in water bath. To the mixture was added benzoyl chloride (92 mg). The mixture was stirred for 30 minutes. To the reaction mixture was added a saturated aqueous solution of sodium bicarbonate. The mixture was extracted with ethyl acetate. The organic layer was washed with water and a saturated aqueous solution of sodium chloride, subsequently, dried and then concentrated. The obtained residue was purified by column chromatography on silica gel (ethyl acetate) to give the compound of the present invention (301 mg) having the following physical data.

TLC: Rf 0.35 (ethyl acetate);

NMR(CDCl₃): δ 1.33 (s, 9H), 1.35-1.53 (m, 2H), 2.00-2.23 (m, 2H), 2.33 (s, 3H), 2.98-3.23 (m, 2H), 3.42-3.65 (m, 2H), 3.70-3.89 (m, 1H), 4.52-4.66 (m, 1H), 6.37 (s, 1H), 6.45-6.58 (m, 4H), 6.97-7.05 (m, 2H), 7.12-7.19 (m, 2H), 7.23-7.29 (m, 2H), 7.37-7.46 (m, 5H).

### EXAMPLE 25

### N-(1-benzoylpiperidin-4-yl)-N-4-[([3-t-butyl-1-(4-methylphenyl)-1H-pyrazol-5-yl]aminocarbonyl)amino]phenylacetamide

To a solution of the compound prepared in Example 24 (70 mg) in pyridine (1.0 mL) was added acetyl chloride (0.010 mL) under ice-cooling, and the mixture was stirred for 15 minutes. To the reaction mixture was added water. The mixture was extracted with ethyl acetate. The organic layer was washed with diluted hydrochloric acid, water and a saturated aqueous solution of sodium chloride, subsequently, dried and then concentrated. The obtained residue was washed with mixed solvent of ethyl acetate-hexane (ca. 2:1) to give the compound of the present invention (65 mg) having the following physical data.

TLC: Rf 0.40 (ethyl acetate:methanol=9:1);

NMR(CDCl₃): δ 1.10-1.29 (m, 2H), 1.34 (s, 9H), 1.73 (s, 3H), 1.75-1.95 (m, 2H), 2.34 (s, 3H), 2.62-2.84 (m, 1H), 2.97-3.21 (m, 1H), 3.67-3.87 (m, 1H), 4.45-4.65 (m, 1H), 4.82-4.96 (m, 1H), 6.41 (s, 1H), 6.82-6.94 (m, 3H), 7.13-7.22 (m, 4H), 7.25-7.31 (m, 2H), 7.32-7.47 (m, 4H).

### EXAMPLE 25(1)

### N-(1-benzoylpiperidin-4-yl)-N-4-[([3-t-butyl-1-(4-methylphenyl)-1H-pyrazol-5-yl]aminocarbonyl)amino]phenylmethanesulfonamide

Using methanesulfonyl chloride instead of acetyl chloride, the compound of the present invention having the following data was obtained by the same procedure of Example 25.

TLC: Rf 0.54 (ethyl acetate:methanol=9:1);

NMR(CDCl₃): δ 1.21-1.51 (m, 11H), 1.76-2.03 (m, 2H), 2.35 (s, 3H), 2.64-2.84 (m, 1H), 2.92 (s, 3H), 2.96-3.19 (m, 1H), 3.69-3.95 (m, 1H), 4.27-4.43 (m, 1H), 4.50-4.73 (m, 1H), 6.38 (s, 1H), 7.04-7.45 (m, 14H), 7.84 (s, 1H).

### EXAMPLE 26

### N-4-[(1-benzoylpiperidin-4-yl)(methyl)amino]phenyl-N′-[3-t-butyl-1-(4-methylphenyl)-1H-pyrazol-5-yl]urea

To a solution of the compound prepared in Example 24 (100 mg) in acetonitrile (2.5 mL) were added 35% aqueous solution of formaldehyde (74 mg) and sodium cyanoborohydride (18.6 mg). The mixture was stirred. To the mixture was added acetic acid (0.013 mL). The mixture was stirred for 1.5 hours at room temperature. To the reaction mixture was added a saturated aqueous solution of sodium bicarbonate. The mixture was extracted with ethyl acetate. The organic layer was washed with water and a saturated aqueous solution of sodium chloride, subsequently, dried and concentrated. The obtained residue was purified by column chromatography on silica gel (acetone:hexane=1:4→1:1) to give the compound of the present invention (88 mg) having the following physical data.

TLC: Rf 0.39 (ethyl acetate);

NMR(CDCl₃): δ 1.33 (s, 9H), 1.62-1.93 (m, 4H), 2.33 (s, 3H), 2.71-2.84 (m, 4H), 2.94-3.22 (m, 1H), 3.66-4.00 (m, 2H), 4.74-4.98 (m, 1H), 6.37 (s, 1H), 6.44-6.55 (m, 2H), 6.68-6.78 (m, 2H), 7.00-7.11 (m, 2H), 7.13-7.20 (m, 2H), 7.22-7.29 (m, 2H), 7.38-7.46 (m, 5H).

### BIOLOGICAL EXAMPLES

It was proven by the following Examples that the compounds of the present invention have p38 αMAP kinase inhibitory activity.

The whole operation using the conventional method is performed according to a fundamental biological technique. Also, as shown below, the measurement method of the present invention is a method wherein enhancement of measurement precision and/or improvement of measurement sensitivity were made in order to evaluate the compounds of the present invention.

Details of such experimental methods were shown below.

(1) Study on p38 αMAP Kinase Inhibitory Activity

Using activation transcription factor 2 (hereinafter abbreviated as ATF-2) which is a substrate for p38 αMAP kinase, the inhibitory action of the compounds of the present invention was investigated on the phosphorylation by a recombinant human p38 αMAP kinase.

[Experimental Method]

A kinase buffer (25 mM Tris-HCl (pH 7.5), 5 mM β-glycerophosphate, 2 mM dithiothreitol, 0.1 mM Na₃VO₄, 10 mM MgCl₂) containing a recombinant human p38 αMAP kinase was added to a 384-well plate (5 μL)(6.25 μg protein/well) for fluorescence measurement. After addition of a kinase buffer (5 μL) containing the compound of the present invention, the resulting mixture was incubated at room temperature for 20 minutes. A substrate mixture (5 μL) of biotinylated ATF2 of 5 μg/mL (Upstate Biotechnology #14-432), adenosine triphosphate (90 μmol/L)(Sigma #FL-AAS) and anti-phosphorylated ATF2 antibody (20-fold dilution)(Cell Signaling Technology #9221L) prepared separately was added thereto, and enzyme reaction was carried out at 30° C. for 30 minutes. After the reaction, Herpes buffer (5 μL) containing 0.25% BSA and 100 mM EDTA was added to stop the enzyme reaction. The amount of a complex of the phosphorylated ATF2 and anti-phosphorylated ATF2 antibody produced by the reaction was measured using an Alpha Screen™ Rabbit Detection kit (Packard #6760607).

The p38 αMAP kinase inhibitory activity, which is the effect of the compound of the present invention, was calculated as an inhibition rate (%) according to the following equation:
Inhibition rate (%)={(A_{C} minus A_{X})/(A_{C} minus A_{B})}×100
wherein A_{B} is a measured value without addition of the enzyme; A_{C} is a measured value with addition of the enzyme in the absence of a test compound; and A_{X} is a measured value with addition of the enzyme in the presence of a test compound.

Inhibition rate of compounds with each concentration was calculated, and a value indicating 50% inhibition (IC₅₀) was determined from the inhibition curve.

As a result, it was confirmed that the compound of the present invention has p38 MAP kinase inhibitory activity. For example, the IC₅₀ values of the compounds in Examples 4(1), 13(1), 13(2) and 17 were 2.9 nM, 5.6 nM, 2.5 nM and 3.8 nM, respectively.

(2) Inhibitory Activity Against TNF-α Production Using Human Cell Lines

Using THP-1 which is a human monocyte cell line, the inhibitory effect of the compound of the present invention against TNF-α production stimulated by lipopolysaccharide (LPS) was studied.

[Experimental Method]

Each 50 μL of lipopolysaccharide (LPS; Difco #3120-25-0) prepared to a concentration of 40 ng/mL using RPMI-1640 medium containing 10% fetal calf serum (hereinafter abbreviated as RPMI-1640) and RPMI-1640 containing the compound of the present invention was added to a 96-well plate for cell culture. One hundred μL of the cell suspension of THP-1 (Dainippon Pharmaceutical Co., Ltd, #06-202) prepared to a cell density of 2×10⁶ cells/mL using RPMI-1640 was added and cultured for 90 minutes at 37° C. in an incubator (5% CO₂, 95% air). After completion of the reaction, the culture medium supernatant was recovered and the amount of produced TNF-α was measured using an ELISA kit (Invitrogen, #850090192).

The inhibitory activity against TNF-α production, which is the effect of the compound of the present invention, was calculated as an inhibition rate (%) by the following equation:
Inhibition rate (%)={(A_{C} minus A_{X})/(A_{C} minus A_{B})}×100
wherein A_{B} is a measured value without LPS induction; A_{C} is a measured value with LPS induction in the absence of a test compound; and A_{X} is a measured value with LPS induction in the presence of a test compound. Inhibition rate of compounds with each concentration was calculated, and a value indicating 50% inhibition (IC₅₀) was determined from the inhibition curve.

As a result, the compound of the present invention showed the inhibitory activity against TNF-α production. For example, the IC₅₀ values of the compounds described in Examples 4 (1), 13(1), 13(2) and 17 were 2.3 nM, 35 nM, 21 nM and 17 nM, respectively.

(3) Rat Cytokine-Production Model

The in vivo effect of the compound of the present invention was studied on TNF-α production induced by lipopolysaccharide (LPS) in rats.

[Experimental Method]

A medium containing the compound of the present invention was orally administered to male Lew mice (Charles River Japan, Inc.), and after 2.0 hours, lipopolysaccharide (LPS, 055:B5, Difco) was intravenously administered at the dose of 10 μg/kg (5 animals/group). Only a medium was orally administered to a control group (5 animals). Ninety minutes after the LPS treatment, heparinized blood collection was performed via the abdominal cava vein under anesthesia with ether, and blood plasma was obtained by centrifugation (12,000 rpm, 3 minutes, 4° C.). The obtained blood plasma sample was stored at −80° C. until it was used. TNF-α in the blood plasma was measured using an ELISA kit from Genzyme/Techne (#10516).

The inhibitory activity of the compound of the present invention against TNF-α production was calculated as an inhibition rate (%) according to the following equation:
Inhibition rate (%)={(A_{C} minus A_{X})/A_{C}}×100
wherein A_{C} is a measured value in case where no test compound was administered under LPS induction, and A_{X} is a measured value in case where a test compound was administered under LPS induction.

The results showed that the compound of the present invention has inhibitory activity against TNF-α production. For example, the compound of Example 4(1) of the present invention at the dose of 10 mg/kg showed an inhibition of 72.8% against in vivo TNF-α production induced by LPS stimulation.

(4) Evaluation of Human CYP3A4 Induction Activity

[Experimental Method]

HepG2 cells were cultured in a 5% CO₂ incubator at 37° C., using a medium [MEM(+)] prepared by adding 1/100-fold amount of non-essential amino acids for MEM Eagle's medium (100×, ICN, #1681049), Antibiotic-Antimycotic (100×, Gibco, #15240-096), L-glutamine 200 mM (100×, Gibco, #25030-081) and 1/10-fold amount of fetal bovine serum (Sigma, #F9423) to a minimum essential medium Eagle (Mod.) with Earle's salts without L-glutamine. The medium was exchanged once every 2 to 3 days. About one-fifth of the cells which were cultured to a confluent state were passaged once a week. HepG2 cells which were cultured to almost confluent state in a 225 cm² culture flask were inoculated to a 24-well plate (Iwaki, #3820-024) to a concentration of 5×10⁴ cells/MEM(+) of 500 μL/well. The cells were cultured in a 5% CO₂ incubator at 37° C. for two days, and a transduction mentioned below was performed. Self-prepared hPXR vector (10 ng), CYP3A4 vector (200 ng) and pRL-TK vector (200 ng) were added to MEM (100 μL) per well in the 24-well plate, and to the resulting solution was added a previously prepared Tfx™-20 reagent (0.75 μL, Promega, #E2391, prepared according to the Instruction Manual). The mixture was admixed by making it upside-down several times, and then allowed to stand at room temperature for 15 minutes (a mixed solution of DNA and liposome). The two-day cultured cells were washed once with PBS(−)(1 mL/well), and added with the prepared DNA and liposome mixture solution (100 μL). After cultivation at 37° C. in a 5% CO₂ incubator for one hour, MEM(+)(440 μL/well) and a test compound (adjusted to 10-fold concentration of the final concentration with MEM(+) containing 1% DMSO; 60 μL/well) were added thereto. Culturing was performed at 37° C. in a 5% CO₂ incubator for two days. The cultured cells were washed once with PBS(−)(1 mL/well) two days after the addition of test compound, and a passive lysis buffer (PLB; 100 μL/well) was added. The mixture was allowed to stand at room temperature 15 or more minutes (cell lysis solution). Twenty μL/well of the prepared cell lysis solution was transferred to a 96-well white plate (Perkin Elmer, #23300), and using a luminometer (Berthold Japan, Microlumat LB96P), each chemical luminescence was measured for 2 to 14 seconds after addition of luciferase assay reagent II [(LARII), 100 μL/well)] and for 2 to 14 seconds after addition of Stop & Glo reagent, 100 μL/well). The preparation and operation procedure of the attached reagents (PLB, LARII and Stop & Glo reagent) was followed according to the Instruction Manual of Dual-Luciferase Reporter Assay System, Promega, #E1910).

The CYP3A4 induction activity was calculated by setting a rise in transcription activity of CYP3A4 to 100% when using rifampicin (10 μmol/L) as a positive control drug.

The results revealed that the compound of the present invention has almost no activity of CYP3A4 induction. For example, the compound of Example 13(1) did not show CYP3A4 induction activity at all at a concentration of 10 μM.

(5) Evaluation of Human CYP2C9 Inhibitory Activity

The CYP2C9 inhibitory activity of the compound of the present invention was evaluated by enhancing measurement precision and improving measurement sensitivity on the basis of Sato et. al method [Pharmacokinetics, Xenobio. Metabol. and Dispos., 16(2), 115-126 (2001)].

The results revealed that the CYP2C9 inhibitory activity of the compound of the present invention was very weak. For example, the compound of Example 13(1) showed only IC50 value of 21 μM.

### FORMULATION EXAMPLES

### FORMULATION EXAMPLE 1

N-[3-t-butyl-1-(4-methylphenyl)-1H-pyrazol-5-yl]-N′-(4-{[3-(2-methylphenyl)-4-oxotetrahydropyrimidin-1(2H)-yl]methyl}phenyl)urea (5.0 kg), carboxymethylcellulose calcium (disintegrator)(0.2 kg), magnesium stearate (lubricant)(0.1 kg) and microcrystalline cellulose (4.7 kg) were admixed in a conventional manner, and tabletted to obtain 100,000 tablets containing an active ingredient of 50 mg/tablet.

### FORMULATION EXAMPLE 2

N-[3-t-butyl-1-(4-methylphenyl)-1H-pyrazol-5-yl]-N′-(4-{[3-(2-methylphenyl)-4-oxotetrahydropyrimidin-1(2H)-yl]methyl}phenyl)urea (2.0 kg), mannitol (20 kg), and distilled water (500 L) were admixed in a conventional manner, filtered with a dust filter, filled in ampoules (5 ml each), and heat-sterilized in an autoclave to obtain 100,000 ampoules containing an active ingredient of 20 mg/ampoule.

### INDUSTRIAL APPLICABILITY

Since the compounds represented by formula (I) of the present invention, or their salts, N-oxides or hydrates, or prodrugs thereof have a low toxicity, they can be used as raw materials for medicaments. Also, they are useful as an agent for the prevention and/or treatment of cytokine-mediated diseases such as articular rheumatism and the like, because they have p38 MAP kinase inhibitory activity.

1. A compound represented by formula (I): 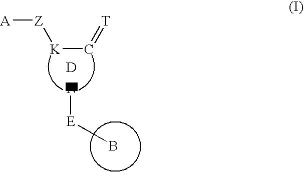
wherein
A represents a hydrogen atom, an optionally substituted cyclic group, an optionally substituted aliphatic hydrocarbon group or an optionally protected amino group;
ring B represents an optionally substituted cyclic group;
E represents a spacer having 1 to 4 atom(s) in its main chain;
K represents a carbon atom or a nitrogen atom;
Z represents a bond, an oxygen atom, a sulfur atom, —NR^{Z}— or —N(SO₂R^{ZZ})—;
R^{Z} represents a hydrogen atom, an optionally substituted cyclic group or an optionally substituted aliphatic hydrocarbon group;
R^{ZZ} represents an optionally substituted cyclic group or an optionally substituted aliphatic hydrocarbon group;
—C(=T)- represents —C(═O)—, —C(═S)— or an optionally substituted methylene group; and
ring D represents an optionally further substituted heterocyclic ring containing at least one nitrogen atom; or its salt, N-oxide or solvate, or a prodrug thereof.

2. The compound according to claim 1, wherein Z is a bond.

3. The compound according to claim 1, wherein Z is an oxygen atom.

4. The compound according to claim 1, wherein —C(=T)- is —C(═O)—.

5. The compound according to claim 1, wherein —C(=T)- is an optionally substituted methylene group.

6. The compound according to claim 1, wherein A is an optionally substituted 5- to 10-membered cyclic group.

7. The compound according to claim 1, wherein A is an optionally substituted C₁₋₈ aliphatic hydrocarbon group or an optionally protected amino group.

8. The compound according to claim 1, wherein the substituent in the A is —NR^{a1}CONR^{a2}R^{a3} in which R^{a1}, R^{a2} and R^{a3} each independently represents a hydrogen atom, an optionally substituted C₁₋₈ alkyl group, an optionally substituted 5- to 10-membered carbocyclic ring, or an optionally substituted 5- to 10-membered heterocyclic ring.

9. The compound according to claim 1, wherein A is

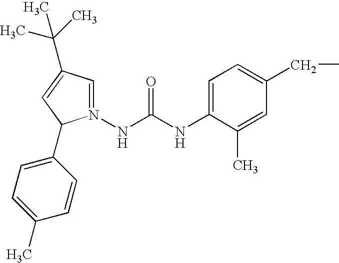

10. The compound according to claim 1, wherein the substituent in the A is —CH₂—CONR^{a1}R^{a2} in which R^{a1} and R^{a2} each independently represents a hydrogen atom, an optionally substituted C₁₋₈ alkyl group, an optionally substituted 5- to 10-membered carbocyclic ring, or an optionally substituted 5- to 10-membered heterocyclic ring.

11. The compound according to claim 1, wherein the ring B is an optionally substituted 5- to 10-membered cyclic group.

12. The compound according to claim 1, wherein the substituent on the ring B is —NR^{a1}CONR^{a2}R^{a3} in which R^{a1}, R^{a2} and R^{a3} each independently represents a hydrogen atom, an optionally substituted C₁₋₈ alkyl group, an optionally substituted 5- to 10-membered carbocyclic ring, or an optionally substituted 5- to 10-membered heterocyclic ring.

13. The compound according to claim 1, wherein the ring B is

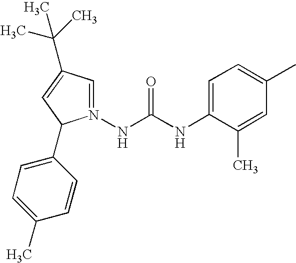

14. The compound according to claim 1, wherein the substituent on the ring B is —CH₂—CONR^{a1}R^{a2} in which R^{a1} and Ra2 each independently represents a hydrogen atom, an optionally substituted C₁₋₈ alkyl group, an optionally substituted 5- to 10-membered carbocyclic ring, or an optionally substituted 5- to 10-membered heterocyclic ring.

15. The compound according to claim 1, wherein K is a nitrogen atom.

16. The compound according to claim 1, wherein K is a carbon atom.

17. The compound according to claim 1, wherein the ring D is an optionally further substituted 6-membered heterocyclic ring containing at least one nitrogen atom.

18. The compound according to claim 4, wherein 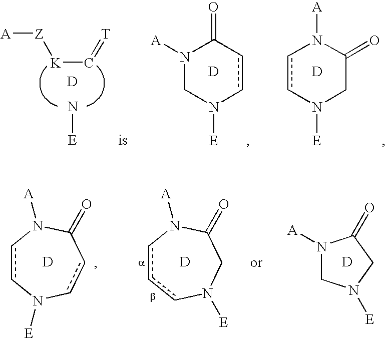
in which [symbol] is a single bond or a double bond, and other symbols have the same meanings as defined in claim 1, provided that the α-bond and β-bond do not simultaneously represent a double bond.

19. The compound according to claim 5, wherein 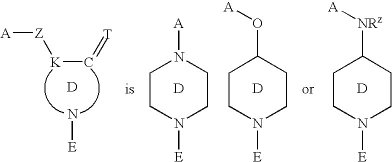
in which all symbols have the same meanings as defined in claim 1.

20. The compound according to claim 1, which is selected from the group consisting of:
(1) N-[3-t-butyl-1-(4-methylphenyl)-1H-pyrazol-5-yl]-N′-(3-{[4-(2-methylbenzoyl)-1-piperazinyl]methyl}phenyl)urea,
(2) N-[3-t-butyl-1-(4-methylphenyl)-1H-pyrazol-5-yl]-N′-(2-methyl-4-{[4-(2-methylbenzoyl)-1-piperazinyl]methyl}phenyl)urea,
(3) N-[3-t-butyl-1-(4-methylphenyl)-1H-pyrazol-5-yl]-N′-(3-chloro-4-{[4-(2-methylbenzoyl)-1-piperazinyl]methyl}phenyl)urea,
(4) N-[3-t-butyl-1-(4-methylphenyl)-1H-pyrazol-5-yl]-N′-[4-({4-[3-(dimethylamino)benzoyl]piperazin-1-yl}methyl)-2-methylphenyl]urea,
(5) N-[3-t-butyl-1-(4-methylphenyl)-1H-pyrazol-5-yl]-N′-{4-[(4-{4-[(E)-(hydroxyimino)methyl]benzoyl}piperazin-1-yl)methyl]-2-methylphenyl}urea,
(6) N-[3-t-butyl-1-(4-methylphenyl)-1H-pyrazol-5-yl]-N′-{4-[(4-{4-[(dimethylamino)methyl]benzoyl}piperazin-1-yl)methyl]-2-methylphenyl}urea,
(7) N-[3-t-butyl-1-(4-methylphenyl)-1H-pyrazol-5-yl]-N′-(2,6-dimethyl-4-{[4-(2-methylbenzoyl)piperazin-1-yl]methyl}phenyl)urea,
(8) N-[3-t-butyl-1-(4-methylphenyl)-1H-pyrazol-5-yl]-N′-(2-fluoro-4-{[4-(2-methylbenzoyl)piperazin-1-yl]methyl}phenyl)urea,
(9) N-[3-t-butyl-1-(4-methylphenyl)-1H-pyrazol-5-yl]-N′-{2-methyl-4-[(4-{4-[(4-methylpiperazin-1-yl)methyl]benzoyl}piperazin-1-yl)methyl]phenyl}urea,
(10) N-[3-t-butyl-1-(4-methylphenyl)-1H-pyrazol-5-yl]-N′-(2-methyl-5-{[4-(2-methylbenzoyl)piperazin-1-yl]methyl}phenyl)urea,
(11) N-{4-[(1-benzoylpiperidin-4-yl)oxy]-2-methylphenyl}-N′-[3-t-butyl-1-(4-methylphenyl)-1H-pyrazol-5-yl]urea,
(12) N-[3-t-butyl-1-(4-methylphenyl)-1H-pyrazol-5-yl]-N′-{4-[(4-{4-[(dimethylamino)methyl]benzoyl}piperazin-1-yl)methyl]-2-fluorophenyl}urea,
(13) N-[3-t-butyl-1-(4-methylphenyl)-1H-pyrazol-5-yl]-N′-{2-fluoro-4-[(4-{4-[(4-methylpiperazin-1-yl)methyl]benzoyl}piperazin-1-yl)methyl]phenyl}urea,
(14) N-[3-t-butyl-1-(4-methylphenyl)-1H-pyrazol-5-yl]-N′-{3-[(4-{4-[(dimethylamino)methyl]benzoyl}piperazin-1-yl)methyl]-2-methylphenyl}urea,
(15) N-[3-t-butyl-1-(4-methylphenyl)-1H-pyrazol-5-yl]-N′-{2-methyl-3-[(4-{4-[(4-methylpiperazin-1-yl)methyl]benzoyl}piperazin-1-yl)methyl]phenyl}urea,
(16) N-[3-t-butyl-1-(4-methylphenyl)-1H-pyrazol-5-yl]-N′-{5-[(4-{4-[(dimethylamino)methyl]benzoyl}piperazin-1-yl)methyl]-2-methylphenyl}urea,
(17) N-[3-t-butyl-1-(4-methylphenyl)-1H-pyrazol-5-yl]-N′-{2-methyl-5-[(4-{4-[(4-methylpiperazin-1-yl)methyl]benzoyl}piperazin-1-yl)methyl]phenyl}urea,
(18) N-[3-t-butyl-1-(4-methylphenyl)-1H-pyrazol-5-yl]-N′-(2-fluoro-5-{[4-(2-methylbenzoyl)piperazin-1-yl]methyl}phenyl)urea,
(19) N-[3-t-butyl-1-(4-methylphenyl)-1H-pyrazol-5-yl]-N′-(2-chloro-5-{[4-(2-methylbenzoyl)piperazin-1-yl]methyl}phenyl)urea,
(20) N-[3-t-butyl-1-(4-methylphenyl)-1H-pyrazol-5-yl]-N′-(2-methoxy-5-{[4-(2-methylbenzoyl)piperazin-1-yl]methyl}phenyl)urea,
(21) N-[3-t-butyl-1-(4-methylphenyl)-1H-pyrazol-5-yl]-N′-{2-methyl-4-[(1-{4-[(4-methylpiperazin-1-yl)methyl]benzoyl}piperidin-4-yl)oxy]phenyl}urea,
(22) N-[3-t-butyl-1-(4-methylphenyl)-1H-pyrazol-5-yl]-N′-{2-fluoro-5-[(4-{4-[(4-methylpiperazin-1-yl)methyl]benzoyl}piperazin-1-yl)methyl]phenyl}urea,
(23) N-[3-t-butyl-1-(4-methylphenyl)-1H-pyrazol-5-yl]-N′-{2-methyl-4-[(4-{3-[(4-methylpiperazin-1-yl)methyl]benzoyl}piperazin-1-yl)methyl]phenyl}urea,
(24) N-[3-t-butyl-1-(4-methylphenyl)-1H-pyrazol-5-yl]-N′-{2-chloro-5-[(4-{4-[(4-methylpiperazin-1-yl)methyl]benzoyl}piperazin-1-yl)methyl]phenyl}urea, and
(25) N-[3-t-butyl-1-(4-methylphenyl)-1H-pyrazol-5-yl]-N′-{2-methyl-4-[(4-{2-[(4-methylpiperazin-1-yl)methyl]benzoyl}piperazin-1-yl)methyl]phenyl}urea.

21. A pharmaceutical composition containing a compound represented by formula (I): 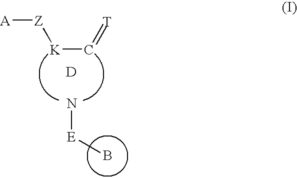
wherein all symbols have the same meanings as defined in claim 1, or its salt, N-oxide or solvate, or a prodrug thereof.

22. The composition according to claim 21, which is a p38 MAP kinase inhibitor.

23. The composition according to claim 21, which is a TNF-α production inhibitor.

24. The composition according to claim 21, which is an agent for prevention and/or treatment of cytokine-mediated disease.

25. The composition according to claim 24, wherein the cytokine-mediated disease is inflammatory disease, cardiovascular disease, respiratory disease and/or bone disease.

26. The composition according to claim 24, wherein the cytokine-mediated disease is central nervous system disease, urinary disease, metabolic disease, endocrine disease, infectious disease and/or cancerous disease.

27. The composition according to claim 25, wherein the inflammatory disease is arthritis rheumatism.

28. A combination medicine comprising a compound of claim 1, its salt, N-oxide or solvate, or a prodrug thereof, and one or two or more compound(s) selected from the group consisting of a non-steroidal anti-inflammatory agent, a disease modifying anti-rheumatic agent, an anticytokine protein preparation, a cytokine inhibitor, an immunomodulator, a steroidal agent, an adhesion molecule inhibitor, an elastase inhhibitor, a cannabinoid-2 receptor stimulant, a prostaglandin, a prostaglandin synthase inhibitor, a phosphodiesterase inhibitor and a metalloproteinase inhibitor.

29. A method for prevention and/or treatment of diseases caused by p38 MAP kinase in a mammal, which comprises administering an effective amount of a compound of claim 1, or its salt, N-oxide or solvate, or a prodrug thereof to a mammal.

30. Use of a compound of claim 1, its salt, N-oxide or solvate, or a prodrug thereof for the preparation of an agent for prevention and/or treatment of diseases caused by p38 MAP kinase.
